SEQUENCE LISTING (63 sequences)

SEQ ID NO: 1 - DNA, 20 bp, synthetic construct
  misc_feature 1..20: note = Synthetic polynucleotide
  source 1..20: mol_type = other DNA
tggcgcgcacccacggcctg

SEQ ID NO: 2 - DNA, 15 bp, synthetic construct
  misc_feature 1..15: note = Synthetic polynucleotide
  source 1..15: mol_type = other DNA
tcctggcggggaagt

SEQ ID NO: 3 - DNA, 12 bp, synthetic construct
  misc_feature 1..12: note = Synthetic polynucleotide
  source 1..12: mol_type = other DNA
cctggatgggaa

SEQ ID NO: 4 - DNA, 24 bp, synthetic construct
  misc_feature 1..24: note = Synthetic polynucleotide
  source 1..24: mol_type = other DNA
cctggatgggaattcccatccagg

SEQ ID NO: 5 - DNA, 24 bp, synthetic construct
  misc_feature 1..24: note = Synthetic polynucleotide
  source 1..24: mol_type = other DNA
cctggatgggaacttaccgctgca

SEQ ID NO: 6 - DNA, 24 bp, synthetic construct
  misc_feature 1..24: note = Synthetic polynucleotide
  source 1..24: mol_type = other DNA
ttagggttagggttagggttaggg

SEQ ID NO: 7 - DNA, 21 bp, synthetic construct
  misc_feature 1..21: note = Synthetic polynucleotide
  source 1..21: mol_type = other DNA
ctcctattgggggtttcctat

SEQ ID NO: 8 - skipped

SEQ ID NO: 9 - DNA, 25 bp, synthetic construct
  misc_feature 1..25: note = Synthetic polynucleotide
  source 1..25: mol_type = other DNA
tttagggttagggttagggttaggg

SEQ ID NO: 10 - DNA, 15 bp, synthetic construct
  misc_feature 1..15: note = Synthetic polynucleotide
  source 1..15: mol_type = other DNA
tcctggaggggaacc

SEQ ID NO: 11 - DNA, 14 bp, synthetic construct
  misc_feature 1..14: note = Synthetic polynucleotide
  source 1..14: mol_type = other DNA
cctggaggggaagt

SEQ ID NO: 12 - DNA, 10 bp, synthetic construct
  misc_feature 1..10: note = Synthetic polynucleotide
  source 1..10: mol_type = other DNA
cctggagggg

SEQ ID NO: 13 - DNA, 18 bp, synthetic construct
  misc_feature 1..18: note = Synthetic polynucleotide
  source 1..18: mol_type = other DNA
tccttcctggaggggaag

SEQ ID NO: 14 - DNA, 19 bp, synthetic construct
  misc_feature 1..19: note = Synthetic polynucleotide
  source 1..19: mol_type = other DNA
tcctatcctggaggggaag

SEQ ID NO: 15 - DNA, 24 bp, synthetic construct
  misc_feature 1..24: note = Synthetic polynucleotide
  source 1..24: mol_type = other DNA
tcctatcctatcctggaggggaag

SEQ ID NO: 16 - DNA, 15 bp, synthetic construct
  misc_feature 1..15: note = Synthetic polynucleotide
  source 1..15: mol_type = other DNA
tcctggaggggaagt

SEQ ID NO: 17 - DNA, 15 bp, synthetic construct
  misc_feature 1..15: note = Synthetic polynucleotide
  source 1..15: mol_type = other DNA
tcctggatgggaagt

SEQ ID NO: 18 - DNA, 24 bp, synthetic construct
  misc_feature 1..24: note = Synthetic polynucleotide
  source 1..24: mol_type = other DNA
ttcccatccaggcctggatgggaa

SEQ ID NO: 19 - DNA, 24 bp, synthetic construct
  misc_feature 1..24: note = Synthetic polynucleotide
  source 1..24: mol_type = other DNA
cttaccgctgcacctggatgggaa

SEQ ID NO: 20 - DNA, 20 bp, synthetic construct
  misc_feature 1..20: note = Synthetic polynucleotide
  source 1..20: mol_type = other DNA
gggggggggggggggggggg

SEQ ID NO: 21 - DNA, 22 bp, synthetic construct
  misc_feature 1..22: note = Synthetic polynucleotide
  source 1..22: mol_type = other DNA
tgactgtgaaggttagagatga

SEQ ID NO: 22 - DNA, 15 bp, synthetic construct
  misc_feature 1..15: note = Synthetic polynucleotide
  source 1..15: mol_type = other DNA
tcctggaggggttgt

SEQ ID NO: 23 - DNA, 18 bp, synthetic construct
  misc_feature 1..18: note = Synthetic polynucleotide
  source 1..18: mol_type = other DNA
tgctcctggaggggttgt

SEQ ID NO: 24 - DNA, 10 bp, synthetic construct
  misc_feature 1..10: note = Synthetic polynucleotide
  source 1..10: mol_type = other DNA
cctggcgggg

SEQ ID NO: 25 - DNA, 18 bp, synthetic construct
  misc_feature 1..18: note = Synthetic polynucleotide
  source 1..18: mol_type = other DNA
cctcaatagggtgagggg

SEQ ID NO: 26 - DNA, 24 bp, synthetic construct
  misc_feature 1..24: note = Synthetic polynucleotide
  source 1..24: mol_type = other DNA
tcgtcgttttgtcgttttgtcgtt

SEQ ID NO: 27 - DNA, 19 bp, synthetic construct
  misc_feature 1..19: note = Synthetic polynucleotide
  source 1..19: mol_type = other DNA
tcgtatcctggaggggaag

SEQ ID NO: 28 - DNA, 19 bp, synthetic construct
  misc_feature 1..19: note = Synthetic polynucleotide
  source 1..19: mol_type = other DNA
taatatcctggaggggaag

SEQ ID NO: 29 - DNA, 18 bp, synthetic construct
  misc_feature 1..18: note = Synthetic polynucleotide
  source 1..18: mol_type = other DNA
cctatcctggaggggaag

SEQ ID NO: 30 - DNA, 22 bp, synthetic construct
  misc_feature 1..22: note = Synthetic polynucleotide
  source 1..22: mol_type = other DNA
gggtgggtgggtattaccatta

SEQ ID NO: 31 - DNA, 18 bp, synthetic construct
  misc_feature 1..18: note = Synthetic polynucleotide
  source 1..18: mol_type = other DNA
tgggcggttcaaccttca

SEQ ID NO: 32 - DNA, 16 bp, synthetic construct
  misc_feature 1..16: note = Synthetic polynucleotide
  source 1..16: mol_type = other DNA
cctcaagcttgagggg

SEQ ID NO: 33 - DNA, 2298 bp, synthetic construct
  misc_feature 1..2298: note = Synthetic polynucleotide
  source 1..2298: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctcgatctgaattcggtaccacgcgtgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctagcaggtaagtgccgtgtgtggttcccgcgggcctggcctctttacgggttatggcccttgcgtgccttgaattactgacactgacatccactttttctttttctccacaggtatcgattgaattccaccatgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaatgaaagatggagatctgcggcctcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggaaccccactccctctctgcgcgctcgctcgctcactgaggccgggcgaccaaaggtcgcccgacgcccgggctttgcccgggcggcctcagtgagcgagcgagcgcgcag

SEQ ID NO: 34 - DNA, 2394 bp, synthetic construct
  misc_feature 1..2394: note = Synthetic polynucleotide
  source 1..2394: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctcgatctgaattcggtacctttagggttagggttagggttagggaaaaatttagggttagggttagggttagggaaaaatttagggttagggttagggttagggaaaaaggtaccacgcgtgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctagcaggtaagtgccgtgtgtggttcccgcgggcctggcctctttacgggttatggcccttgcgtgccttgaattactgacactgacatccactttttctttttctccacaggtatcgattgaattccaccatgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaatgaaagatggagatctgcggcctcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggaaccccactccctctctgcgcgctcgctcgctcactgaggccgggcgaccaaaggtcgcccgacgcccgggctttgcccgggcggcctcagtgagcgagcgagcgcgcag

SEQ ID NO: 35 - DNA, 90 bp, synthetic construct
  misc_feature 1..90: note = Synthetic polynucleotide
  source 1..90: mol_type = other DNA
tttagggttagggttagggttagggaaaaatttagggttagggttagggttagggaaaaatttagggttagggttagggttagggaaaaa

SEQ ID NO: 36 - DNA, 3295 bp, synthetic construct
  misc_feature 1..3295: note = Synthetic polynucleotide
  source 1..3295: mol_type = other DNA
tgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagcggcctcggcctctgcataaataaaaaaaattagtcagccatgagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgctctagaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgc

SEQ ID NO: 37 - DNA, 3484 bp, synthetic construct
  misc_feature 1..3484: note = Synthetic polynucleotide
  source 1..3484: mol_type = other DNA
tgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagcggcctcggcctctgcataaataaaaaaaattagtcagccatgagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgctctagaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacaggtacctttagggttagggttagggttagggaaaaatttagggttagggttagggttagggaaaaatttagggttagggttagggttagggaaaaatgcagcggtaagttcccatccaggttttttgcagcggtaagttcccatccaggttttttgcagcggtaagttcccatccaggtttttgctagctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgc

SEQ ID NO: 38 - DNA, 3601 bp, synthetic construct
  misc_feature 1..3601: note = Synthetic polynucleotide
  source 1..3601: mol_type = other DNA
tgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagcggcctcggcctctgcataaataaaaaaaattagtcagccatgagcttggcccattgcatacgttgtatccatatcataatatgtacatttatattggctcatgtccaacattaccgccatgttgacattgattattgactagtcctggatgggaaaaaaacctggatgggaaaaaaacctggatgggaaaaaaaacttccccgccaggatttttacttccccgccaggatttttacttccccgccaggatttttactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgctctagaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacaggtacctttagggttagggttagggttagggaaaaatttagggttagggttagggttagggaaaaatttagggttagggttagggttagggaaaaatgcagcggtaagttcccatccaggttttttgcagcggtaagttcccatccaggttttttgcagcggtaagttcccatccaggtttttgctagctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgc

SEQ ID NO: 39 - DNA, 177 bp, synthetic construct
  misc_feature 1..177: note = Synthetic polynucleotide
  source 1..177: mol_type = other DNA
tttagggttagggttagggttagggaaaaatttagggttagggttagggttagggaaaaatttagggttagggttagggttagggaaaaatgcagcggtaagttcccatccaggttttttgcagcggtaagttcccatccaggttttttgcagcggtaagttcccatccaggttttt

SEQ ID NO: 40 - DNA, 111 bp, synthetic construct
  misc_feature 1..111: note = Synthetic polynucleotide
  source 1..111: mol_type = other DNA
cctggatgggaaaaaaacctggatgggaaaaaaacctggatgggaaaaaaaacttccccgccaggatttttacttccccgccaggatttttacttccccgccaggattttt

SEQ ID NO: 41 - DNA, 2693 bp, synthetic construct
  misc_feature 1..2693: note = Synthetic polynucleotide
  source 1..2693: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 42 - DNA, 2876 bp, synthetic construct
  misc_feature 1..2876: note = Synthetic polynucleotide
  source 1..2876: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaggtacctttagggttagggttagggttagggaaaaatttagggttagggttagggttagggaaaaatttagggttagggttagggttagggaaaaatgcagcggtaagttcccatccaggttttttgcagcggtaagttcccatccaggttttttgcagcggtaagttcccatccaggtttttctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 43 - DNA, 15 bp, synthetic construct
  misc_feature 1..15: note = Synthetic polynucleotide
  source 1..15: mol_type = other DNA
tcctgagcttgaagt

SEQ ID NO: 44 - DNA, 24 bp, synthetic construct
  misc_feature 1..24: note = Synthetic polynucleotide
  source 1..24: mol_type = other DNA
ttattattattattattattatta

SEQ ID NO: 45 - DNA, 4369 bp, synthetic construct
  misc_feature 1..4369: note = Synthetic polynucleotide
  source 1..4369: mol_type = other DNA
aaagcttcccggggggatctgggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacctaggcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccggactctaaggtaaatataaaatttttaagtgtataatgtgttaaactactgattctaattgtttctctcttttagattccaacctttggaactgaattccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgctaggcctcacctgcgatctcgatgctttatttgtgaaatttgtgatgctattgctttatttgtaaccattataagctgcaataaacaagttaacaacaacaattgcattcattttatgtttcaggttcagggggaggtgtgggaggttttttaaactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctag

SEQ ID NO: 46 - DNA, 4450 bp, synthetic construct
  misc_feature 1..4450: note = Synthetic polynucleotide
  source 1..4450: mol_type = other DNA
aaagcttcccggggggatctgggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacctaggcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccggactctaaggtaaatataaaatttttaagtgtataatgtgttaaactactgattctaattgtttctctcttttagattccaacctttggaactgaattccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgctaggcctcacctgcgatctcgatgctttatttgtgaaatttgtgatgctattgctttatttgtaaccattataagctgcaataaacaagttaacaacaacaattgcattcattttatgtttcaggttcagggggaggtgtgggaggttttttaaatcgattggcgcgcacccacggcctgaaaaatggcgcgcacccacggcctgaaaaatggcgcgcacccacggcctgaaaaaactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctag

SEQ ID NO: 47 - DNA, 4465 bp, synthetic construct
  misc_feature 1..4465: note = Synthetic polynucleotide
  source 1..4465: mol_type = other DNA
aaagcttcccggggggatctgggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacctaggcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccggactctaaggtaaatataaaatttttaagtgtataatgtgttaaactactgattctaattgtttctctcttttagattccaacctttggaactgaattccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgctaggcctcacctgcgatctcgatgctttatttgtgaaatttgtgatgctattgctttatttgtaaccattataagctgcaataaacaagttaacaacaacaattgcattcattttatgtttcaggttcagggggaggtgtgggaggttttttaaatcgattttagggttagggttagggttagggaaaaatttagggttagggttagggttagggaaaaatttagggttagggttagggttagggaaaaaactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctag

SEQ ID NO: 48 - DNA, 4465 bp, synthetic construct
  misc_feature 1..4465: note = Synthetic polynucleotide
  source 1..4465: mol_type = other DNA
aaagcttcccggggggatctgggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacctaggtttagggttagggttagggttagggaaaaatttagggttagggttagggttagggaaaaatttagggttagggttagggttagggaaaaacctaggcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccggactctaaggtaaatataaaatttttaagtgtataatgtgttaaactactgattctaattgtttctctcttttagattccaacctttggaactgaattccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgctaggcctcacctgcgatctcgatgctttatttgtgaaatttgtgatgctattgctttatttgtaaccattataagctgcaataaacaagttaacaacaacaattgcattcattttatgtttcaggttcagggggaggtgtgggaggttttttaaactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctag

SEQ ID NO: 49 - DNA, 6940 bp, synthetic construct
  misc_feature 1..6940: note = Synthetic polynucleotide
  source 1..6940: mol_type = other DNA
cagcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagcggcctcggcctctgcataaataaaaaaaattagtcagccatgagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgctctagaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 50 - DNA, 7257 bp, synthetic construct
  misc_feature 1..7257: note = Synthetic polynucleotide
  source 1..7257: mol_type = other DNA
cagcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagcggcctcggcctctgcataaataaaaaaaattagtcagccatgagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgctctagaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacaggtacctttagggttagggttagggttagggaaaaatttagggttagggttagggttagggaaaaatttagggttagggttagggttagggaaaaatttagggttagggttagggttagggaaaaatttagggttagggttagggttagggaaaaaccctaaccctaaccctaaccctaaattttttccctaaccctaaccctaaccctaaattttttccctaaccctaaccctaaccctaaattttttccctaaccctaaccctaaccctaaattttttccctaaccctaaccctaaccctaaattttttgctagctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 51 - DNA, 4369 bp, synthetic construct; identical to SEQ ID NO: 45
  misc_feature 1..4369: note = Synthetic polynucleotide
  source 1..4369: mol_type = other DNA

SEQ ID NO: 52 - DNA, 4450 bp, synthetic construct; identical to SEQ ID NO: 46
  misc_feature 1..4450: note = Synthetic polynucleotide
  source 1..4450: mol_type = other DNA

SEQ ID NO: 53 - DNA, 4425 bp, synthetic construct
  misc_feature 1..4425: note = Synthetic polynucleotide
  source 1..4425: mol_type = other DNA
aaagcttcccggggggatctgggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacctaggcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccggactctaaggtaaatataaaatttttaagtgtataatgtgttaaactactgattctaattgtttctctcttttagattccaacctttggaactgaattccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgctaggcctcacctgcgatctcgatgctttatttgtgaaatttgtgatgctattgctttatttgtaaccattataagctgcaataaacaagttaacaacaacaattgcattcattttatgtttcaggttcagggggaggtgtgggaggttttttaaatcgattggcgcgcacccacggcctgaaaaatggcgcgcacccacggcctgaaaaaactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctag

SEQ ID NO: 54 - DNA, 4465 bp, synthetic construct; identical to SEQ ID NO: 47
  misc_feature 1..4465: note = Synthetic polynucleotide
  source 1..4465: mol_type = other DNA

SEQ ID NO: 55 - DNA, 4405 bp, synthetic construct
  misc_feature 1..4405: note = Synthetic polynucleotide
  source 1..4405: mol_type = other DNA
aaagcttcccggggggatctgggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacctaggcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccggactctaaggtaaatataaaatttttaagtgtataatgtgttaaactactgattctaattgtttctctcttttagattccaacctttggaactgaattccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgctaggcctcacctgcgatctcgatgctttatttgtgaaatttgtgatgctattgctttatttgtaaccattataagctgcaataaacaagttaacaacaacaattgcattcattttatgtttcaggttcagggggaggtgtgggaggttttttaaatcgattttagggttagggttagggttagggaaaaaactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctag

SEQ ID NO: 56 - DNA, 4465 bp, synthetic construct; identical to SEQ ID NO: 48
  misc_feature 1..4465: note = Synthetic polynucleotide
  source 1..4465: mol_type = other DNA

SEQ ID NO: 57 - DNA, 4435 bp, synthetic construct
  misc_feature 1..4435: note = Synthetic polynucleotide
  source 1..4435: mol_type = other DNA
aaagcttcccggggggatctgggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacctaggcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccggactctaaggtaaatataaaatttttaagtgtataatgtgttaaactactgattctaattgtttctctcttttagattccaacctttggaactgaattccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgctaggcctcacctgcgatctcgatgctttatttgtgaaatttgtgatgctattgctttatttgtaaccattataagctgcaataaacaagttaacaacaacaattgcattcattttatgtttcaggttcagggggaggtgtgggaggttttttaaatcgatgctagatgttagcgtaaaaagctagatgttagcgtaaaaagctagatgttagcgtaaaaaactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctag

SEQ ID NO: 58 - DNA, 6940 bp, synthetic construct; identical to SEQ ID NO: 49
  misc_feature 1..6940: note = Synthetic polynucleotide
  source 1..6940: mol_type = other DNA

SEQ ID NO: 59 - DNA, 7257 bp, synthetic construct; identical to SEQ ID NO: 50
  misc_feature 1..7257: note = Synthetic polynucleotide
  source 1..7257: mol_type = other DNA

SEQ ID NO: 60 - DNA, 15 bp, synthetic construct
  misc_feature 1..15: note = Synthetic polynucleotide
  source 1..15: mol_type = other DNA
gctagatgttagcgt

SEQ ID NO: 61 - skipped

SEQ ID NO: 62 - DNA, 18 bp, synthetic construct
  misc_feature 1..18: note = Synthetic
  source 1..18: mol_type = other DNA
ttagggttagggttaggg

SEQ ID NO: 63 - skipped

(19) United States
(12) Patent Application Publication
(10) Pub. No.: US 2024/0384295 A1
(43) Pub. Date: Nov. 21, 2024

(54) COMPOSITIONS AND METHODS FOR INHIBITING VIRAL VECTOR-INDUCED INFLAMMATORY RESPONSES

(71) Applicant: President and Fellows of Harvard College, Cambridge, MA (US)

(72) Inventors: Ying Kai Chan, Cambridge, MA (US); George M. Church, Cambridge, MA (US)

(73) Assignee: President and Fellows of Harvard College, Cambridge, MA (US)

(21) Appl. No.: 18/629,140

(22) Filed: Apr. 8, 2024

Related U.S. Application Data
(63) Continuation of application No. 16/762,356, filed on May 7, 2020, now Pat. No. 11,981,911.
(60) Provisional application No. 62/595,433, filed on Dec. 6, 2017.
(60) Provisional application No. 62/583,449, filed on Nov. 8, 2017.

(51) Int. Cl. C12N 15/86; C12N 15/117

(52) U.S. Cl. CPC C12N 15/86; C12N 15/117; C12N 2710/10343

(57) ABSTRACT

Provided herein, in some embodiments, are recombinant viral genomes comprising an inhibitory oligonucleotide that reduces inflammation for use, for example, in gene therapy.

### RELATED APPLICATIONS

This application is a national stage filing under 35 U.S.C. § 371 of international application number PCT/US2018/059756, filed Nov. 8, 2018, which claims the benefit under 35 U.S.C. § 119 (e) of U.S. provisional application No. 62/583,449, filed Nov. 8, 2017 and U.S. provisional application No. 62/595,433, filed Dec. 6, 2017, each of which is incorporated by reference herein in its entirety.

### REFERENCE TO A SEQUENCE LISTING SUBMITTED AS A TEXT FILE VIA EFS-WEB

This application contains a Sequence Listing which has been submitted in ASCII format via EFS-Web and is hereby incorporated by reference in its entirety. Said ASCII copy, created on Oct. 6, 2022, is named H049870653US02-SUBSEQ-KVC and is 139,521 bytes in size.

### FEDERALLY SPONSORED RESEARCH

This invention was made with government support under Grant No. HG008525 awarded by the National Institutes of Health. The government has certain rights in the invention.

### BACKGROUND

Viruses have evolved to become highly efficient at nucleic acid delivery to specific cell types while avoiding immunosurveillance by an infected host. These properties make viruses attractive gene-delivery vehicles, or vectors, for gene therapy. Several types of viruses, including retrovirus, adenovirus, adeno-associated virus (AAV), and herpes simplex virus, have been modified in the laboratory for use in gene therapy (Robbins, PD et al. Pharmol Ther, 1998;80(1):35-47). Nonetheless, viral vectors have been shown to elicit an inflammatory response.

### SUMMARY

Provided herein, in some embodiments, are viral molecular therapy vectors that inhibit nucleic acid-mediated inflammatory responses while boosting expression of a desired therapeutic molecule (e.g., a therapeutic gene of interest). The vectors of the present disclosure include a recombinant viral genome linked in cis to an inhibitory oligonucleotide sequence that prevents virally-induced production of proinflammatory cytokines. In some embodiments, the inhibitory oligonucleotide inhibits nucleic acid-mediated activation of toll-like receptors and/or inhibits nucleic acid-mediated toll-like receptor (TLR) signaling (e.g., TLR9). Surprisingly, inclusion of the inhibitory oligonucleotide in the viral genome not only inhibits the inflammatory response, but it also increases transduction efficiency and/or efficacy of the therapeutic nucleotide sequence and/or expression levels of expression products encoded by nucleic acids. as appropriate. Thus, the amount of recombinant viral genome needed to be therapeutically effective is less than the amount needed with conventional viral vector delivery systems that do not include an inhibitory oligonucleotide.

Thus, some aspects of the present disclosure provide recombinant viral genomes comprising a therapeutic nucleic acid (e.g., DNA encoding a gene (e.g., Cas9) or gene fragment (e.g., a replacement exon of interest) and an inhibitory oligonucleotide that inhibits the production of proinflammatory cytokines. Also provided herein, in some aspects, are methods comprising administering to a subject a recombinant viral genome that comprises a therapeutic nucleic acid (e.g., DNA) and an inhibitory oligonucleotide that inhibits the production of proinflammatory cytokines. In some embodiments, the recombinant viral genomes are administered intramuscularly. In other embodiments, the recombinant viral genomes are administered intravenously. In some embodiments, the recombinant viral genomes are administered to the eye (e.g., intravitreally).

Other aspects of the present disclosure provide recombinant viral genomes comprising a therapeutic nucleotide sequence and an inhibitory oligonucleotide that inhibits nucleic acid-sensing TLR activation and/or signaling. Also provided herein, in some aspects, are methods comprising administering to a subject a recombinant viral genome comprising a therapeutic nucleotide sequence and an inhibitory oligonucleotide that inhibits nucleic acid-sensing TLR activation and/or signaling.

In some embodiments, the TLR is TLR9. In some embodiments, the inhibitory oligonucleotide binds to the TLR. In other embodiments, the inhibitory oligonucleotide binds to inflammatory nucleic acids. In some embodiments, the inflammatory nucleic acids comprise CpG oligodeoxynucleotides. In some embodiments, the inhibitory oligonucleotide comprises a CCx(not-C)(not-C)xxGGG (SEQ ID NO: 63) motif, wherein x is any nucleic acid.

### BRIEF DESCRIPTION OF THE DRAWINGS

FIGS. 1A-1C includes data showing the impact of exemplary inhibitory oligonucleotides on TLR9 activation in vitro. HEK293-TLR9 reporter cells were treated with the indicated constructs (single-stranded DNA with a phosphorothioate backbone) at the indicated concentrations for 18 hours. A 50-μl sample of cell supernatant was incubated with 100 μl HEK-Blue Detection and then analyzed on a plate reader for absorbance at 639 nm. The data shown are mean±s.d. of n=3 technical replicates. FIG. 1A: TLR9 activating nucleic acid (ODN 2006) linked to each of the indicated sequences on the X-axis was tested in the HEK293-TLR9 reporter system. The left graph in FIG. 1A shows results using 0.5 μM of the constructs. The right graph in FIG. 1A shows results using 5 μM of the constructs. FIG. 1B: TLR9 activating nucleic acid (ODN 2006) linked through a AAAAA (SEQ ID NO: 8) linker to each of the indicated sequences on the X-axis was tested in the HEK293-TLR9 reporter system. FIG. 1C: TLR9 activating nucleic acid (ODN 2006) linked to each of the sequences indicated on the X-axis was tested in the HEK293-TLR9 reporter system.

FIGS. 2A-2B show muscle tissue data relating to the impact of three copies of the telomere oligonucleotide (“3xtelomere” SEQ ID NO: 35) on the inflammatory response elicited by a linked self-complementary adeno-associated virus (scAAV) genome and on AAV transgene expression in infected mice. FIG. 2A: shows data relating to the impact of indicated constructs or control saline on expression of proinflammatory cytokines, using qRT-PCR. Adult C57BL/6 mice were infected with indicated AAV2 viruses (10¹¹ vg per mouse) by intramuscular injection in the quadricep. 2 hours (h) later, the animals were euthanized and a piece of the quadricep was analyzed for indicated gene expression by qRT-PCR. Saline injection was set to 1-fold expression for each gene. Data shown are mean±s.d. of n=4 or 5 mice per condition. FIG. 2B: shows data relating to the impact of indicated AAV vectors on GFP expression. Adult C57BL/6 mice were infected with indicated AAV2 viruses similar to FIG. 2A and a piece of the quadricep was analyzed for GFP gene expression by qRT-PCR 28 d later. scAAV-cGFP infection was set to 1-fold expression for GFP. Data shown are n=5 mice per condition. Each triangle represents an animal and mean values are indicated.

FIGS. 3A-3C includes liver tissue data relating to the impact of three copies of telomere (“3xtelomere” SEQ ID NO: 35) and three copies of c41 (3×c41) oligonucleotides on the inflammatory response elicited by a linked self-complementary adeno-associated virus (scAAV) in infected mice. All three panels show data relating to the impact of indicated constructs or control saline on expression of proinflammatory cytokines, using qRT-PCR. Adult C57BL/6 mice were infected with indicated AAV2 viruses (10¹¹ vg per mouse) by tail vein injections. 2 h later, the animals were euthanized and a piece of the liver was analyzed for indicated gene expression by qRT-PCR. Saline injection was set to 1-fold expression for each gene. FIG. 3A: data shown are mean±s.d. of n=3 mice per condition. FIGS. 3B and 3C: data shown are mean±s.d. of n=4 mice per condition. *P<0.05 (unpaired t-test) compared to saline condition. N.s.: not significant (P>0.05). FIG. 3D includes liver tissue data relating to the impact of the inhibitory oligonucleotide sequence or a control sequence on the inflammatory response elicited by a linked scAAV genome in infected mice. Adult C57BL/6 mice were infected with indicated AAV2 viruses and a piece of the liver was analyzed for indicated gene expression by qRT-PCR. Data shown are mean±s.d. of n=5 mice per condition except n=3 mice for scAAV-eGFP-3xcontrol. *P<0.05 (unpaired t-test) compared to saline condition. N.s.: not significant (P>0.05).

FIG. 3E includes liver tissue data relating to the impact of three copies of telomere oligonucleotide (“3xtelomere” SEQ ID NO: 35) on scAAV transgene expression in infected mice. Adult C57BL/6 mice were infected with indicated AAV2 viruses (10¹¹ vg per mouse) by tail vein injections. 14 d later, the animals were euthanized and a piece of the liver was analyzed for GFP gene expression by qRT-PCR. scAAV-eGFP injection was set to 1-fold expression for GFP. Data shown are n=4 mice per condition. Each triangle represents an animal and mean values are indicated.

FIG. 4 is a schematic representation of a single-stranded AAV vector encoding GFP (ssAAV-eGFP). Two engineered forms are shown. ssAAV-eGFP-5xtelomere carries an insertion of 5 copies of telomere oligonucleotide (5×SEQ ID NO: 9) with AAAAA (SEQ ID NO: 8) linkers, followed by reverse complementary sequences of another 5 copies with AAAAA linkers (in anti-sense orientation). ssAAV-eGFP-3xtelo3xINH18 carries an insertion of 3 copies of telomere oligonucleotide (3×SEQ ID NO: 9) with AAAAA (SEQ ID NO: 8) linkers, followed by reverse complementary sequences of 3 copies of INH-18 (3×SEQ ID NO: 5) with AAAAA (SEQ ID NO: 8) linkers (in anti-sense orientation) (“3xtelo3xINH18 SEQ ID NO 39). LpA: polyA signal.

FIG. 5 includes a graph showing inflammatory responses to various inhibitory oligonucleotides in HEK293-TLR9 reporter cells in vitro. HEK293-TLR9 reporter cells were treated with 0.02 μM of the indicated oligonucleotides (see Example 1) for 18 hours. A 50 μl sample of cell supernatant was incubated with 150 μl HEK-Blue Detection and then analyzed on a plate reader for absorbance at 630 nm. Cells were treated with (1) a random 24 nucleotide strand (“control”) that does not contain CG, (2) ODN 2006 (SEQ ID NO: 26) fused to ODN TTAGGG (SEQ ID NO: 6) (in cis), or (3) ODN 2006 co-administered with ODN TTAGGG (in trans). Data shown are mean±s.d. of n=3 technical replicates.

FIGS. 6A-6B includes data showing the impact of exemplary inhibitory oligonucleotides on TLR7 and TLR2 activation in vitro. HEK293-TLR7 or HEK293-TLR2 reporter cells were treated with or without a high concentration of 5 μM of the indicated constructs (single-stranded DNA with a phosphorothioate backbone), along with the appropriate TLR7 stimulant (1 μg/ml of Gardiquimod) or TLR2 stimulant (100 ng/ml of FSL-2), for 18 hours. TTAGGG in FIGS. 6A-6B refers to ODN TTAGGG (SEQ ID NO: 6). A 50-μl sample of cell supernatant was incubated with 100 μl HEK-Blue Detection and then analyzed on a plate reader for absorbance at 639 nm. The data shown are mean±s.d. of n=3 technical replicates. FIG. 6A shows results using HEK293-TLR7 reporter cells and FIG. 6B shows results using HEK293-TLR2 reporter cells.

FIGS. 7A-7E show the effects of incorporation of three copies of the telomere sequence (“3xtelomere” SEQ ID NO: 35) into a self-complementary AAV vector encoding human factor IX on innate immune response and transgene expression in vivo in liver tissue. FIG. 7A shows a schematic diagram of vector organization of scAAV-FIX (the original “wild-type” vector, SEQ ID NO: 33) and scAAV-FIX-3xtelomere (SEQ ID NO: 34). AAV vectors were packaged in an AAV8 capsid. FIGS. 7B and 7C show innate immune responses in mouse liver assayed by qPCR 2 h after intravenous administration of indicated vector at indicated dose. PBS injection was set to 1-fold expression for each gene. Data shown are mean±s.e.m. of n=5-7 animals per condition. *p<0.05 by two-tailed Mann-Whitney test and compared against PBS condition. ns, not significant, p>0.05. FIGS. 7D and 7E show human factor IX levels in plasma of mice at indicated time points. Data shown are mean±s.d. of n=5-8 animals per condition. **p<0.005 by two-tailed Mann-Whitney test. ns, not significant, p>0.05. ITR, inverted terminal repeat; TTR, transthyretin promoter; hFIX, human factor IX; bGH, bovine growth hormone poly(A) signal; TRS, terminal resolution site.

FIGS. 8A-8D include data showing the effects of multiple copies of inhibitory oligonucleotides (multiple copies of telomere (multiple copies of SEQ ID NO: 9) and multiple copies of INH-18 (multiple copies of SEQ ID NO: 5) on AAV-induced T cell reactivity and T cell infiltration. FIG. 8A includes data where mice received indicated doses of AAVrh32.33 vectors (single-stranded AAV) via intramuscular injections and 21 d later, splenocytes were subject to IFN-γ ELISPOT assays to quantify CD8+ T cell responses to an immunodominant epitope of rh32.33 capsid. Representative images of the ELISPOT well for animals with median responses for each condition are shown. The dotted line (50 SFU/10⁶ splenocytes) indicates the cutoff for a positive T cell response in this assay. ssAAV-eGFP is provided as SEQ ID NO: 36. ssAAV-eGFP-3xtelo3xINH18 is provided as SEQ ID NO: 37. FIGS. 8B-8C show the number of CD8+ T cells and CD8+ Granzyme B+ T cells in the muscle sections (four fields examined per sample) for PBS and 1×10¹⁰ vg rh32.33 vectors 21 dpi. (FIG. 8D) Representative images of GFP expression by immunohistochemistry staining in muscle sections at 21 dpi. Scale bar, 50 μm. n=5-10 animals per condition as indicated. *p<0.05 and **p<0.005 by two-tailed Mann-Whitney test. ns, not significant, p>0.05. SFU, spot forming units.

FIGS. 9A-9B show characterization of AAV8 vectors used in a pig study. ssAAV-eGFP vector is provided as SEQ ID NO: 36. ssAAV-eGFP-3xtelo3xINH18 vector is provided as SEQ ID NO: 37. FIG. 9A is a silver staining comparison of 1×10¹⁰ vg AAV8 vectors. FIG. 9B is a series of transmission electron microscopy images showing negatively stained AAV, and representative empty particles are shown with white arrows. Representative images are shown for each vector. Scale bar: 100 nm.

FIGS. 10A-10C show that engineered vector evades photoreceptor pathology and immune responses in subretinal-injected pig eyes. ssAAV-eGFP is provided as SEQ ID NO: 36. ssAAV-eGFP-3xtelo3xINH18 is provided as SEQ ID NO: 37. FIG. 10A shows immunohistochemical images of ONL of retina 6 weeks after subretinal injections. Outer segments of cone photoreceptors were visualized by anti-red-green cone opsin staining. Scale bars, 10 μm. FIG. 10B shows microglia proliferation and activation in the retina indicated by anti-Iba1 staining. Scale bars, 50 μm. FIG. 10C shows cytotoxic T cell infiltration into the retina indicated by anti-CD8 staining. Scale bars, 50 μm. Each animal is indicated by an identification number and the two images are from the two treated eyes of each animal. ONL, outer nuclear layer: Iba1, ionized calcium-binding adaptor protein 1.

FIG. 11 shows that engineered vector comprising an inhibitory oligonucleotide evades photoreceptor pathology in subretinal-injected pig eyes. Immunohistochemical images of retina 6 weeks after subretinal injections. Cone photoreceptors were visualized by anti-human cone arrestin staining. Scale bars, 50 μm. Each animal is indicated by an identification number and the two images are from the two treated eyes of each animal. Regions shown for AAV8-injected eyes are GFP+, but GFP signal from the right half of each image was digitally removed to allow better visualization of arrestin staining. ssAAV-eGFP is provided as SEQ ID NO: 36. ssAAV-eGFP-3xtelo3xINH18 is provided as SEQ ID NO: 37.

FIG. 12 shows a schematic representation of a single-stranded AAV vector encoding GFP (ssAAV-eGFP). Two engineered forms are shown. ssAAV-eGFP-3xtelo3xINH18 (SEQ ID NO: 37) carries an insertion of 3 copies of telomere oligonucleotide (3×SEQ ID NO: 9) with AAAAA (SEQ ID NO: 8) linkers, followed by reverse complementary sequences of 3 copies of INH-18 (3×SEQ ID NO: 5) with AAAAA linkers (in anti-sense orientation, i.e., anti-sense orientation of SEQ ID NO: 8) in the 3′ untranslated region (between the polyA signal and right ITR). ssAAV-eGFP-double (SEQ ID NO: 38) carries the same insertion, but additionally carries an insertion of 3 copies of 4084-F (3×SEQ ID NO: 3) oligonucleotide with AAAAA (SEQ ID NO: 8) linkers, followed by reverse complementary sequences of 3 copies of 2088 (3×SEQ ID NO: 2) oligonucleotide with AAAAA linkers (in anti-sense orientation, i.e., anti-sense orientation of SEQ ID NO: 8) in the 5′ untranslated region (between the left ITR and the promoter). LpA: polyA signal.

FIG. 13 shows a schematic representation of a different single-stranded AAV vector encoding GFP (ssAAV-eGFP-WPRE). An engineered form is shown. ssAAV-eGFP-WPRE-3xtelo3xINH18 (SEQ ID NO: 42) carries an insertion of 3 copies of telomere oligonucleotide (3×SEQ ID NO: 9) with AAAAA (SEQ ID NO: 8) linkers, followed by reverse complementary sequences of 3 copies of INH-18 (3×SEQ ID NO: 5) with AAAAA linkers (in anti-sense orientation, i.e., anti-sense orientation of SEQ ID NO: 8) in the 3′ untranslated region (between the polyA signal and right ITR). WPRE: Woodchuck Hepatitis Virus Posttranscriptional Regulatory Element. LpA: polyA signal.

### DETAILED DESCRIPTION

Despite recent advances, the effectiveness of viral vectors as gene delivery vehicles has been limited, in part, due to vector-induced inflammation. Thus, clinically, gene therapy with viral vectors often includes systemic treatment with an immunosuppressive agent. Such immunosuppressive and anti-inflammatory drugs, however, can compromise the patient's immune system during treatment and patients often still develop neutralizing antibodies or T cells to exogenous biological materials (e.g., against AAV capsid), precluding future re-administration or leading to destruction of transduced cells.

Provided herein, in some embodiments, are recombinant viral genomes that may be used as delivery vehicles, without inducing a substantial inflammatory response. In some embodiments, the recombinant viral genomes induce less of an inflammatory response than would otherwise be induced without the presence of the inhibitory oligonucleotide. The recombinant viral genomes of the present disclosure include an inhibitory oligonucleotide that inhibits production of proinflammatory cytokines, thus, inhibiting the inflammatory response. In some embodiments, the inhibitory oligonucleotide inhibits an inflammatory response induced by the toll-like receptor (TLR) pathway, for example, by inhibiting (preventing) TLR activation and/or inhibiting TLR signaling.

### Inflammatory Response to Viral Vectors

Viral vectors are often studied in experimental and clinical models as agents for gene therapy. Recent generations of viral vectors have the majority of viral genes removed and result in vectors with a large carrying capacity, reduced host immune responses and improved gene transfer efficiency. Some viral vectors, such as adenovirus vectors and adeno-associated virus vectors, however, still activate innate immune responses following administration in vivo. Unlike the adaptive response, the innate response to viral vectors is independent of immunological memory and is caused by recognition of conserved features of microbes, commonly termed pattern-associated molecular patterns (PAMPs). This response results in inflammation of transduced tissues and can reduce viral transduction efficiency. Viral infection can activate a number of signaling pathways following cell entry that ultimately lead to expression of inflammatory (proinflammatory) genes. Various cytokines, chemokines and leukocyte adhesion molecules are induced by the viral vector in a wide range of cell types providing a molecular basis for the inflammatory properties of these vectors. See Liu, Q. et al. Gene Therapy 2003:10:935-940.

### Toll-Like Receptor Signaling Pathway

One of the signaling pathways activated following in vivo administration (in a subject) of viral vectors is the toll-like receptor (TLR) signaling pathway. TLRs are immune pattern-recognition receptors that detect pathogens and damaged cells. For example, TLR9 is well-known, and TLR9 amino acid sequences can be found in publically-available gene databases, such as GenBank and UnitProtKB. For example, the amino acid sequence of wild-type human TLR9 can be identified as UniProtKB entryQ9NR96 (TLR9_Human).

TLR9 is generally located on endosomal membranes in immune cells. TLR9 is an exemplary nucleic acid-sensing TLRs that detects exogenous nucleic acids (inflammatory nucleic acids) that have entered a cell (see, e.g. Takeda, K et al., Semin Immunol. 2004;16(1):3-9; Lee, J et al. Proc Natl Acad Sci USA. 2011;108(34):14055-60). Nucleic acids recognized by TLR9 include those that originate from bacteria, viruses or even endogenous nucleic acids. ‘Nucleic acid-sensing TLRs’ are TLRs that can bind to nucleic acids, such as ssRNA, dsRNA, and DNA containing unmethylated CpG (cytosine-phosphate-guanine) motifs. This binding typically results in the dimerization of TLR and activation of TLR signaling, which results in the production of (expression of and/or activation of) proinflammatory molecules, such as proinflammatory cytokines. In particular, TLR9 recognizes nucleic acids with unmethylated CpGs (Kumagai, Y, et al. Adv Drug Deliv Rev. 2008;60(7)795-804).

As used herein, unless otherwise specified, ‘inflammatory nucleic acids’ are nucleic acids that activate TLR signaling (e.g., bind to TLR to activate TLR signaling). In some embodiments, the inflammatory nucleic acids comprise deoxycytidyl-deoxyguanosine (CpG) oligodeoxynucleotides. CpG oligodeoxynucleotides are sequences that comprise at least one unmethylated CpG motif and activate an immune response. See, e.g., Krieg, AM et al. Nature. 1995: 374(6522):546-9. In some embodiments, the inflammatory nucleic acids (e.g., CpG oligodeoxynucleotides) activate TLR9 signaling (e.g., by binding to TLR9). An exemplary inflammatory nucleic acid is provided in Example 1 (ODN 2006).

TLR signaling results in an inflammatory response characterized by gene expression of antiviral molecules and proinflammatory cytokines, including type I interferons and NF-kB (p25-RelA complex) target genes. Thus, TLR signaling may be used as means to determine the impact of an inhibitory oligonucleotide on the inflammatory response. In some embodiments, a TLR reporter cell line may be used to assess the inhibitory nature of an oligonucleotide, for example, by determining the level of cytokine (e.g. IL6, CXCL 10 and/or TNF) production, which reflects the level of TLR signaling (see, e.g., Example 1). As an example, recognition of unmethylated CpG motifs in exogenous DNA activates TLR9, and TLR9 signaling leads to an increase in expression of proinflammatory cytokines, including IL6, CXCL 10, and/or TNF. See, e.g., Krieg AM Nat Rev Drug Discov. 2006;5(6):471-84. In some embodiments, the level of a particular cytokine is measured using quantitative PCR with primers targeting the cytokine of interest (see, e.g., Examples 3 and 4). Additional methods of measuring cytokine levels include enzyme-linked immunosorbent assay (ELISA) and Western blot analysis with an anti-cytokine antibody.

Inhibition of an inflammatory response may be measured as a decrease in TLR signaling. For example, a decrease in cytokine activity level or expression level (e.g., a 2-fold, 5-fold, 10-fold, 50-fold reduction) relative to a control may indicate inhibition (partial or complete inhibition) of the inflammatory response (see, e.g., Examples 3 and 4).

### Inhibitory oligonucleotides

An inhibitory oligonucleotide is an oligonucleotide that, when co-delivered in vivo with another nucleic acid (such as a viral genome, a single-stranded RNA, or a single-stranded DNA), inhibits the production of proinflammatory cytokines, relative to proinflammatory cytokine production in the absence of the inhibitory oligonucleotide. See, e.g., Stunz, LL et al. Eur J Immunol. 2002;32(5):1212-22; Lenert, P et al. DNA Cell Biol. 2003;22(10):621-31; Lenert, P et al. Arthritis Res Ther. 2009;11(3):R79; Lenert, PS et al. Arthritis Res Ther. 2006;8(1):203: Kaminski, JJ et al. J Immunol. 2013;191(7):3876-83; Shirota, H et al. J Immunol. 2005;174(8):4579-83; Peter, M et al. Immunology. 2008;123(1):118-28. The inhibitory oligonucleotides of the present disclosure comprise at least two nucleotides covalently linked together, and in some instances, may contain phosphodiester bonds (e.g., a phosphodiester “backbone”). In some instances, the oligonucleotide may contain phosphorothioate bonds (e.g., a phosphorothioate backbone). The length of an inhibitory oligonucleotide may vary, but is should be understood that the length of an inhibitory oligonucleotide is typically 4 to 200 nucleotides. In some embodiments, an inhibitory oligonucleotide has a length of 4 to 100 nucleotides. In some embodiments, an inhibitory oligonucleotide has a length of 4 to 10, 4 to 20, 4 to 30, 4 to 50, 4 to 60, 4 to 70, 4 to 80, or 4 to 90 nucleotides. In some embodiments, an inhibitory oligonucleotide bas a length of 5 to 10, 5 to 20, 5 to 30, 5 to 50, 5 to 60, 5 to 70, 5 to 80, 5 to 90, or 5 to 100 nucleotides. In some embodiments, an inhibitory oligonucleotide has a length of 6 to 10, 6 to 20, 6 to 30, 6 to 50, 6 to 60, 6 to 70, 6 to 80, 6 to 90, or 6 to 100 nucleotides. In some embodiments, an inhibitory oligonucleotide has a length of 7 to 10, 7 to 20, 7 to 30, 7 to 50, 7 to 60, 7 to 70, 7 to 80, 7 to 90, or 7 to 100 nucleotides. In some embodiments, an inhibitory oligonucleotide has a length of 8 to 10, 8 to 20, 8 to 30, 8 to 50, 8 to 60, 8 to 70, 8 to 80, 8 to 90, or 8 to 100 nucleotides. In some embodiments, an inhibitory oligonucleotide has a length of 9 to 10, 9 to 20, 9 to 30, 9 to 50, 9 to 60, 9 to 70, 9 to 80, 9 to 90, or 9 to 100 nucleotides. In some embodiments, an inhibitory oligonucleotide has a length of 10 to 10, 10 to 20, 10 to 30, 10 to 50, 10 to 60, 10 to 70, 10 to 80, 10 to 90, or 10 to 100 nucleotides. Inhibitory oligonucleotides may be produced recombinantly or synthetically, for example.

In some embodiments, the inhibitory oligonucleotide comprises or consists of deoxyribonucleotides. Thus, in some embodiments, the inhibitory oligonucleotide is an inhibitory DNA oligonucleotide. In some embodiments, the inhibitory oligonucleotide does not include RNA. It should be understood that the definition of inhibitory oligonucleotides, as provided herein, specifically excludes RNA interference molecules (RNAi), such as short interfering RNA (siRNA) molecules.

In some embodiments, the inhibitory oligonucleotides inhibit the activation of nucleic acid-sensing TLRs. For example, the inhibitory oligonucleotides may act as molecular scavengers and bind to (and sequester) inflammatory nucleic acids, thus preventing the inflammatory nucleic acids from binding the TLR and activating TLR signaling.

In some instances, the inhibitory oligonucleotides may prevent dimerization of a TLR.

In some embodiments, the inhibitory oligonucleotides inhibit TLR signaling (to downstream molecules). For example, the inhibitory oligonucleotides may bind indirectly or directly to a TLR (e.g., TLR9) to block TLR-mediated production of proinflammatory cytokines (e.g., induction of proinflammatory cytokine activity and/or expression). See, e.g. Lenert, PS Mediators Inflamm. 2010;2010:986596; Ohto, U et al. Nature. 2015;520(7549):702-5; Lee, J et al. Proc Natl Acad Sci USA. 2011;108(34):14055-60. In some embodiments, the inhibitor oligonucleotide competes for receptor-mediated endocytosis or phagocytosis. In some embodiments, the inhibitor oligonucleotide inhibits TLR9 trafficking. In some embodiments, the inhibitor oligonucleotide inhibits TLR9 processing into a functionally active product. In some embodiments, the inhibitor oligonucleotide inhibits endosomal acidification or activity of key proteases in endosomes. In some embodiments, the inhibitor oligonucleotide blocks signaling proteins downstream of TLR9.

It should be understood that the term “inhibits” encompasses complete (100%) inhibition and partial (less than 100%) inhibition, otherwise referred to as reduction. Thus, an inhibitory oligonucleotide may reduce nucleic acid-sensing TLR activation and/or signaling by at least 10%, at least 20%, at least 30%, at least 40%, at least 50%, at least 60%, at least 70%, at least 80%, at least 909%, or at least 95%, relative to control (nucleic acid-sensing TLR activation and/or signaling in the absence of the inhibitory oligonucleotide).

In some embodiments, the inhibitory oligonucleotides of the present disclosure inhibit production of proinflammatory cytokines. Non-limiting examples of proinflammatory cytokines include interleukins (e.g., IL-1, IL-6, IL-17 and IL-18), interferons (IFNs, e.g., interferon α (IFNα), interferon β (IFNβ), and interferon γ (IFNγ)), tumor necrosis factors (TNFs) (e.g., TNF-α) and chemokines (e.g., CCL2, CXCL10 and CCL5). In some embodiments, the inhibitory oligonucleotide inhibits IL-6, CXCL10 and/or TNF production. In some embodiments, the inhibitory oligonucleotide inhibits IL-6 production. In some embodiments, the inhibitory oligonucleotide inhibits CXCL10 production. In some embodiments, the inhibitory oligonucleotide inhibits TNF production. As discussed herein, the level of inflammatory cytokine production may be measured using Western blot analysis, quantitative PCR and/or enzyme-linked immunosorbent assay. Other assays for assessing the inflammatory response are known and may be used as provided herein.

In some embodiments, an inhibitory oligonucleotide reduces production of (activity of and/or expression of) proinflammatory cytokines by at least 10%, at least 20%, at least 30%, at least 40%, at least 50%, at least 60%, at least 70%, at least 80%, at least 90%, or at least 95%, relative to control (inflammatory cytokine production in the absence of the inhibitory oligonucleotide).

The inhibitory oligonucleotides of the present disclosure may include different motifs that contribute to anti-inflammatory properties of the inhibitory oligonucleotide. In some embodiments, the inhibitory oligonucleotide comprises at least one CCx(not-C)(not-C)xxGGG (SEQ ID NO: 63) motif, wherein x is any nucleic acid (e.g., A, T, C or G (but not C where specified)). See, e.g., Ashman, RF et al. Int Immunol. 2011; 23(3):203-14. Non-limiting examples of inhibitory oligonucleotides that comprise at least one CCx(not-C)(not-C)xxGGG (SEQ ID NO: 63) motif include ODN 4228 (see, e.g., Ashman, RF et al. Int Immunol. 2011; 23(3):203-14), SEQ ID NO: 2, SEQ ID NO: 3, SEQ ID NO: 4, SEQ ID NO: 5, SEQ ID NO: 7, SEQ ID NO: 16, SEQ ID NO: 17, SEQ ID NO: 18, SEQ ID NO: 19, SEQ ID NO: 21, SEQ ID NO: 22, SEQ ID NO: 23, and SEQ ID NO: 24.

In some embodiments, an inhibitory oligonucleotide comprises at least one TTAGGG motif (SEQ ID NO: 61). For example, an inhibitory oligonucleotide may comprise at least 2, 3, 4, 5, 6, 7, 8, 9 or 10 TTAGGG motifs. In some embodiments, the inhibitory oligonucleotide includes two TTAGGG motifs. In some embodiments, the inhibitory oligonucleotide includes three TTAGGG motifs. In some embodiments, the inhibitory oligonucleotide includes four TTAGGG motifs. Exemplary inhibitory oligonucleotides comprising at least one TTAGGG motif include those identified by SEQ ID NO:6 and/or SEQ ID NO: 9.

In some embodiments, the inhibitory oligonucleotide includes at least one sequence that is identical to a nucleotide sequence of any one of SEQ ID NOS: 1-7, 9-25, or 27-32.

In some embodiments, the inhibitory oligonucleotide includes a nucleotide sequence that is identical to the nucleotide sequence identified by SEQ ID NO: 1. In some embodiments, the inhibitory oligonucleotide includes a nucleotide sequence that is identical to SEQ ID NO: 6. In some embodiments, the inhibitory oligonucleotide includes a nucleotide sequence that is identical to the nucleotide sequence identified by SEQ ID NO: 9.

In some embodiments, the inhibitory oligonucleotide includes multiple tandem repeats of a nucleotide sequence of any one of SEQ ID NOS: 1-7, 9-25, or 27-32. As used herein, unless otherwise noted, tandem repeats are sequences that that follow one another. In some examples, the tandem repeats may be directly next to one another (e.g., TTAGGGTTAGGGTTAGGG (SEQ ID NO: 62) (repeated sequence underlined)). In some examples, the tandem repeats may be separated by another sequence (e.g., a linker sequence) (e.g., TTAGGG-linker-TTAGGG-linker-TTAGGG). In some embodiments, the inhibitory oligonucleotide includes multiple tandem repeat sequences (e.g. two, three, four or five tandem repeats). In some embodiments, the inhibitory oligonucleotide includes multiple tandem repeats (e.g. two, three, four or five repeats) of SEQ ID NO: 1. In some embodiments, the inhibitory oligonucleotide includes multiple tandem repeats (e.g. two, three, four or five repeats) of SEQ ID NO: 9. In some embodiments, the inhibitory oligonucleotide includes multiple tandem repeats (e.g. two, three, four or five repeats) of SEQ ID NO: 6.

In some embodiments, the multiple tandem repeat sequences in an inhibitory oligonucleotide are separated by a linker. The linker may be oriented in the sense or antisense direction. As an example, a linker may be oriented in the sense direction if it is separating multiple tandem repeat sequences that are also oriented in the sense direction. In certain embodiments, a linker is oriented in the antisense direction when it is separating multiple tandem repeat sequences that are also oriented in the antisense direction.

In some embodiments, the linker is a polyA linker (a string of “A” nucleotides). In some embodiments, the polyA linker comprises at least one nucleotide sequence that is identified by SEQ ID NO: 8. In some embodiments, the polyA linker comprises two nucleotide sequences that is identified by SEQ ID NO: 8. In some embodiments, the polyA linker comprises three nucleotide sequences that is identified by SEQ ID NO: 8. For example, an inhibitory oligonucleotide may have three tandem repeats of the sequence in SEQ ID NO: 1 and each of the tandem repeats may be separated by a linker identified by SEQ ID NO: 8. In certain embodiments, the linker comprises at least one nucleotide sequence (e.g., at least 2, 3, 4, 5, 6, 7, 8, 9, or 10 nucleotide sequences) that is the antisense sequence of SEQ ID NO: 8 (i.e., SEQ ID NO: 8 oriented in the antisense direction).

As a non-limiting example, an inhibitory oligonucleotide may have three tandem repeats of the sequence in SEQ ID NO: 9 or SEQ ID NO: 6 and each of the tandem repeats may be separated by a linker identified by SEQ ID NO: 8. In some embodiments, the viral genome comprises a sequence that is at least 90% (e.g., at least 91%, at least 92%, at least 93%, at least 94%, at least 95%, at least 96%, at least 97%, at least 98%, at least 99%, or is 100%) identical to SEQ ID NO: 35. In certain embodiments, a recombinant viral genome is single-stranded and comprises the sequence in SEQ ID NO: 35 that is located upstream (5′) of a therapeutic nucleotide sequence (e.g., Factor IX) (e.g., in the 5′ UTR). In certain embodiments, a recombinant viral genome is single-stranded and comprises the sequence in SEQ ID NO: 35 that is located downstream (3′) of a therapeutic nucleotide sequence (e.g., Factor IX) (e.g., downtream from (3′) a polyA tail linked to the therapeutic nucleic acid, including in the 3′ UTR). In some embodiments, the viral genome comprises a sequence that is at least 90% (e.g., at least 91%, at least 92%, at least 93%, at least 94%, at least 95%, at least 96%, at least 97%, at least 98%, at least 99%, or is 100%) identical to SEQ ID NO: 34.

In some embodiments, the inhibitory oligonucleotide includes a combination of nucleotide sequences of any one of SEQ ID NOS: 1-7, 9-25, or 27-32. For example, the inhibitory oligonucleotide may include at least one copy (e.g., one, two, three, four, five, six or seven copies) of a nucleotide sequence of of any one of SEQ ID NOS: 1-7, 9-25, or 27-32combined with at least one copy (e.g., one, two, three, four, five, six or seven copies) of a nucleotide sequence of any one of SEQ ID NOS: 1-7, 9-25, or 27-32. In some embodiments, the inhibitory oligonucleotide includes three copies of SEQ ID NO: 9 and three copies of SEQ ID NO: 5 (e.g., SEQ ID NO: 39). In some embodiments, the inhibitory oligonucleotide comprises three copies of SEQ ID NO: 3 and three copies of SEQ ID NO: 2. In some embodiments, the inhibitory oligonucleotide includes five copies of SEQ ID NO: 1 and three copies of SEQ ID NO: 9. See also Example 8 and Materials and Methods section of the Examples below.

As discussed in the Examples section, a single expression construct (e.g., ssAAV) may include more than one (e.g., at least 2, at least 3, at least 4, at least 5, at least 6, at least 7, at least 8, at least 9, at least 10, at least 15, or at least 20) type of inhibitory oligonucleotides, one type (one or more copies, e.g., 2, 3, 4 or 5 tandem copies) in the sense orientation and another type (one or more copies, e.g., 2, 3, 4 or 5 tandem copies) in the antisense orientation. Without being bound by a particular theory, inclusion of two different types of inhibitory oligonucleotides may prevent self-annealing between the inhibitory oligonucleotides and formation of unwanted hairpin structures. In some embodiments, a recombinant single-stranded viral genome comprising an inhibitory oligonucleotide in the sense orientation and another inhibitory oligonucleotide in the antisense direction increases the probability that each packaged viral genome comprises at least one inhibitory oligonucleotide in the correct orientation.

As a non-limiting example, an inhibitory oligonucleotide may have three tandem repeats of the sequence in SEQ ID NO: 9 or SEQ ID NO: 6 and three tandem repeats of the sequence in SEQ ID NO: 5. The tandem repeats of the sequence in SEQ ID NO: 9 or SEQ ID NO: 6 may be oriented in the opposite direction of the tandem repeats of the sequence in SEQ ID NO: 5. For example, the tandem repeats of the sequence in SEQ ID NO: 9 or SEQ ID NO: 6 may be oriented in the sense direction and the tandem repeats of the sequence in SEQ ID NO: 5 may be oriented in the antisense direction or vice versa. The tandem repeats of SEQ ID NO: 9 or SEQ ID NO: 6 may be upstream (5′) or downstream (3′) of the tandem repeats of the sequence in SEQ ID NO: 5. Each of the tandem repeats (e.g., repeats of SEQ ID NOS: 5, 6, 9, or any combination thereof) may be separated by a linker identified by SEQ ID NO: 8 oriented in the sense or antisense direction. In some embodiments, the viral genome comprises a sequence that is at least 90% (e.g., at least 91%, at least 92%, at least 93%, at least 94%, at least 95%, at least 96%, at least 97%, at least 98%, at least 99%, or is 100%) identical to SEQ ID NO: 39. In certain embodiments, a recombinant viral genome is single-stranded and comprises the sequence in SEQ ID NO: 39 that is located upstream (5′) (e.g., in the 5′ UTR) of a therapeutic nucleotide sequence. In certain embodiments, a recombinant viral genome is single-stranded and comprises the sequence in SEQ ID NO: 39 that is located downstream (3′) (e.g., downtream from (3′) a polyA tail linked to the therapeutic nucleic acid, including in the 3′ UTR) of a therapeutic nucleotide sequence.

In certain embodiments, an inhibitory oligonucleotide may have three tandem repeats of the sequence in SEQ ID NO: 3 and three tandem repeats of the sequence in SEQ ID NO: 2. The tandem repeats of the sequence in SEQ ID NO: 3 may be oriented in the opposite direction of the tandem repeats of the sequence in SEQ ID NO: 2. For example, the tandem repeats of the sequence in SEQ ID NO: 3 may be oriented in the sense direction and the tandem repeats of the sequence in SEQ ID NO: 2 may be oriented in the antisense direction or vice versa. The tandem repeats of SEQ ID NO: 3 may be upstream (5′) or downstream (3′) of the tandem repeats of the sequence in SEQ ID NO: 2. Each of the tandem repeats (e.g., repeats of SEQ ID NOS: 2, 3, or any combination thereof) may be separated by a linker identified by SEQ ID NO: 8 that is oriented in the sense or antisense direction. In some embodiments, the viral genome comprises a sequence that is at least 90% (e.g., at least 91%, at least 92%, at least 93%, at least 94%, at least 95%, at least 96%, at least 97%, at least 98%, at least 99%, or is 100%) identical to SEQ ID NO: 40. In certain embodiments, a recombinant viral genome is single-stranded and comprises the sequence in SEQ ID NO: 40 that is located upstream (5′) (e.g., in the 5′ UTR) of a therapeutic nucleotide sequence. In certain embodiments, a recombinant viral genome is single-stranded and comprises the sequence in SEQ ID NO: 40 that is located downstream (3′) (e.g., downtream from (3′) a polyA tail linked to the therapeutic nucleic acid, including in the 3′ UTR) of a therapeutic nucleotide sequence.

It should be understood that the present disclosure encompasses the use of any inhibitory oligonucleotide, such as those described herein, as well as inhibitory oligonucleotides that share a certain degree of sequence identity (percent identity) with a reference inhibitory oligonucleotide (e.g., SEQ ID NO:1, SEQ ID NO:6, or SEQ ID NO: 9). Percent identity refers to a relationship between the sequences of two or more polynucleotides (nucleic acids), as determined by comparing the sequences. Identity measures the percent of identical matches between the smaller of two or more sequences with gap alignments (if any) addressed by a particular mathematical model or computer program (e.g., “algorithms”). Identity of related molecules can be readily calculated by known methods. “Percent (%) identity” as it applies to nucleic acid sequences is defined as the percentage of nucleic acid residues in the candidate nucleic acid sequence that are identical with the residues in the nucleic acid sequence of a second sequence after aligning the sequences and introducing gaps, if necessary, to achieve the maximum percent identity. Identity depends on a calculation of percent identity but may differ in value due to gaps and penalties introduced in the calculation. Variants of a particular inhibitory oligonucleotide may have at least 90%, 91%, 92%, 93%, 94%, 95%, 96%, 97%, 98%, 99% but less than 100% sequence identity to that particular reference inhibitory oligonucleotide sequence, as determined by sequence alignment programs and parameters described herein and known to those skilled in the art.

Thus, in some embodiments, the inhibitory oligonucleotide includes at least one sequence that is at least 80% identical to a nucleotide sequence of any one of SEQ ID NOS: 1-7, 9-25, or 27-32. In some embodiments, the inhibitory oligonucleotide includes at least one sequence that is at least 90% identical to a nucleotide sequence of any one of SEQ ID NOS: 1-7, 9-25, or 27-32.

The comparison of sequences and determination of percent identity between two sequences can be accomplished using a mathematical algorithm. Techniques for determining identity are codified in publicly available computer programs. Exemplary computer software to determine homology between two sequences include, but are not limited to, the GCG program package (Devereux, J. et al. Nucleic Acids Research, 12(1): 387, 1984), the BLAST suite (Altschul, S. F. et al. Nucleic Acids Res. 25: 3389, 1997), and FASTA (Altschul, S. F. et al. J. Molec. Biol. 215: 403, 1990). Other techniques include: the Smith-Waterman algorithm (Smith, T.F. et al. J. Mol. Biol. 147: 195, 1981; the Needleman-Wunsch algorithm (Needleman, S.B. et al. J. Mol. Biol. 48: 443, 1970; and the Fast Optimal Global Sequence Alignment Algorithm (FOGSAA) (Chakraborty, A. et al. Sci Rep.3: 1746, 2013).

### Recombinant Viral Genomes

The present disclosure provides recombinant viral genomes in which an (at least one) inhibitory oligonucleotide is included in the viral genome. In some embodiments, two or more inhibitory oligonucleotide is included in the viral genome. Multipe inhibitory oligonucleotides may be located in different locations throughout the viral genome (relative to each other). The viral genomes typically include a therapeutic nucleotide sequence and an inhibitory oligonucleotide. The inhibitory oligonucleotide may be located, for example, in the 3′ untranslated region (UTR) of the viral genome (see, e.g., scAAV-eGFP-3xc41 and scAAV-eGFP-3x telomere in Example 2 described below). In certain embodiments, the inhibitory oligonucleotide is downstream (3′) relative to the therapeutic nucleotide sequence. In certain embodiments, the inhibitory oligocnuleotide is located downtream from (3′) a polyA tail linked to the therapeutic nucleic acid. In some embodiments, the inhibitory oligonucleotide is located in the 5′ UTR of the viral genome. In some embodiments, the inhibitory oligonucleotide is located upstream (5′) of a promoter operably linked to the therapeutic nucleotide sequence. In some instances, the inhibitory oligonucleotide is located upstream (5′) relative to the therapeutic nucleotide sequence. As a non-limiting example, a recombinant viral genome may comprise an inhibitory oligonucleotide located downstream (3′) (e.g., downtream from (3′) a polyA tail linked to the therapeutic nucleic acid, including in the 3′ UTR) and an inhibitory oligonucleotide located upstream (5′) (e.g., in the 5′ UTR) of the therapeutic nucleotide sequence. Surprisingly, the location of the inhibitory oligonucleotide in the viral genome, relative to the promoter, does not impact the inhibitory function of the oligonucleotide. The recombinant viral genome may comprise inflammatory nucleic acids (e.g., CpG oligodeoxynucleotides). The inflammatory nucleic acids may be located anywhere in the viral genome (e.g., the viral ITR, the promoter, the intron, the transgene, the 5′ UTR, the 3′ UTR, etc.). For example, the therapeutic nucleotide sequence may comprise inflammatory nucleic acids (e.g., CpG oligodeoxynucleotide).

The inhibitory oligonucleotides of the present disclosure may be oriented in the sense direction and/or antisense direction in the viral genome. In some embodiments, a viral genome includes 1, 2, 3, 4, or 5 copies of an inhibitory oligonucleotide (e.g., 1, 2, 3, 4, or 5 copies of SEQ ID NO: 9) in the sense direction. In some embodiments, the same viral genome includes 1, 2, 3, 4, or 5 copies of the same inhibitory oligonucleotide (e.g., 1, 2, 3, 4, or 5 of reverse complement of SEQ ID NO:9) or a different oligonucleotide (e.g., reverse complement of SEQ ID NO: 1 or SEQ ID NO:5) in the antisense direction. See also Example 6 and Materials and Methods section of the Examples below.

The recombinant viral genomes, as provided herein, may be used, in some embodiments, to deliver (to a subject) a therapeutic nucleotide sequence of interest (e.g., a therapeutic DNA, a therapeutic RNA, and/or a therapeutic protein encoded by the nucleotide sequence). In some embodiments, the recombinant viral genomes of the present disclosure are gene delivery vectors. Thus, in some embodiments, the therapeutic nucleotide sequence is a gene encoding a therapeutic protein, as discussed elsewhere herein.

A recombinant viral genome, generally, is a viral genome that is not naturally occurring. The viral genomes may be from adeno-associated virus (AAV), adenovirus, herpes simplex virus, varicella, variola virus, hepatitis B, cytomegalovirus, JC polyomavirus, BK polyomavirus, monkeypox virus, Herpes Zoster, Epstein-Barr virus, human herpes virus 7, Kaposi's sarcoma-associated herpesvirus, or human parvovirus B19. Other viral genomes are encompassed by the present disclosure.

In some embodiments, a viral genome is an AAV genome. AAV is a small, non-enveloped virus that packages a single-stranded linear DNA genome that is approximately 5 kb long, and has been adapted for use as a gene transfer vehicle (Samulski, RJ et al., Annu Rev Virol. 2014;1(1):427-51). The coding regions of AAV are flanked by inverted terminal repeats (ITRs), which act as the origins for DNA replication and serve as the primary packaging signal (McLaughlin, SK et al. J Virol. 1988;62(6):1963-73; Hauswirth, WW et al. 1977;78(2):488-99). Both positive and negative strands are packaged into virions equally well and capable of infection (Zhong, L. et al. Mol Ther. 2008;16(2):290-5; Zhou, X et al. Mol Ther. 2008;16(3):494-9; Samulski, RJ et al. J Virol. 1987;61(10):3096-101). In addition, a small deletion in one of the two ITRs allows packaging of self-complementary vectors, in which the genome self-anneals after viral uncoating. This results in more efficient transduction of cells but reduces the coding capacity by half (McCarty, DM et al. Mol Ther. 2008;16(10):1648-56; McCarty, DM et al. Gene Ther. 2001;8(16):1248-54).

In some embodiments, the recombinant viral genomes of the present disclosure include a single-stranded nucleotide sequence. In some embodiments, the viral genome is self-complementary. A self-complementary viral genome is a viral genome that forms an intramolecular double-stranded nucleotide sequence. Examples of methods for making recombinant viral genomes and self-complementary (sc) viral genomes are provided in Example 2 and in the Materials and Methods section of the Examples below.

In some embodiments, the viral genome is a single-stranded nucleotide sequence (e.g., ssAAV). In some embodiments, the single-stranded viral genome does not form an intramolecular double-stranded nucleotide sequence. In certain embodiments, a recombinant viral genome is a single-stranded viral genome comprising an inhibitory oligonucleotide downstream (3′) of the therapeutic nucleotide sequence (e.g., downtream from (3′) a polyA tail linked to the therapeutic nucleic acid, including in the 3′ UTR of the viral genome). In certain embodiments, a recombinant viral genome is a single-stranded viral genome comprising an inhibitory oligonucleotide upstream (5′) of the therapeutic nucleotide sequence (e.g., in the 5′ UTR of the viral genome). As a non-limiting example, a single-stranded viral genome comprising an inhibitory oligonucleotide that is located upstream (5′) of a therapeutic nucleotide sequence (e.g., Factor IX) is provided as SEQ ID NO: 34. In certain embodiments, a recombinant viral genome is a single-stranded viral genome comprising an inhibitory oligonucleotide upstream (5′) of the therapeutic nucleotide sequence (e.g., in the 5′ UTR of the viral genome) and an inhibitory oligonucleotide downstream (3′) of the therapeutic nucleotide sequence (e.g., in the 3′ UTR of the viral genome).

As a non-limiting example, a recombinant viral genome may comprise at least two different inhibitory oligonucleotides located upstream (5′) of a therapeutic nucleotide sequence and at least two different inhibitory oligonucleotides sequences located downstream (3′) of the therapeutic nucleotide sequence. In some instances, all inhibitory oligonucleotides sequences are different in a recombinant viral genome. For example, a recombinant viral genome may comprise inhibitory oligonucleotides that each comprise different multiple tandem repeats of a sequence that is at least 90% identical to a sequence of any one of SEQ ID NOS: 1-7, 9-25, or 27-32. In some instances, an inhibitory oligonucleotide comprises multiple tandem repeats of two different sequences selected from to a sequence of any one of SEQ ID NOS: 1-7, 9-25, or 27-32.

In some instances, a recombinant viral genome comprises 1) an inhibitory oligonucleotide sequence that is at least 90% (e.g., at least 91%, 92%, 93%, 94%, 95%, 96%, 97%, 98%, 99%, or is identical) to SEQ ID NO: 39 located upstream (5′) of a therapeutic nucleotide sequence (e.g., in the 5′ UTR) and 2) an inhibitory oligonucleotide that is at least 90% (e.g., at least 91%, 92%, 93%, 94%, 95%, 96%, 97%, 98%, 99%, or is identical) to SEQ ID NO: 40 that is located downstream (3′) of a therapeutic nucleotide sequence (e.g., in the 3′ UTR). Alternatively, the inhibitory oligonucleotide that is at least 90% identical to SEQ ID NO: 39 may be located downstream (3′) of a therapeutic nucleotide sequence (e.g., in the 3′ UTR) and the inhibitory oligonucleotide that is at least 90% identical to SEQ ID: 40 may be located upstream (5′) of a therapeutic nucleotide sequence (e.g., in the 5′ UTR).

Exemplary methods for making a single-stranded viral genome with limited self-annealing are provided in Example 8 and in the Materials and Methods section of the Examples below.

The recombinant viral genomes of the present disclosure may comprise a therapeutic nucleotide sequence. A therapeutic nucleotide sequence is a nucleotide sequence (e.g., RNA or DNA) that confers a therapeutic benefit or encodes a molecule (e.g., protein) that confers a therapeutic benefit to a subject when administered in vivo. In some embodiments, the therapeutic nucleotide sequence is a therapeutic RNA sequence (e.g., an RNAi molecule). In some embodiments, the therapeutic nucleotide sequence is a therapeutic DNA sequence (e.g. a DNA aptamer that binds a target). In some embodiments, the therapeutic nucleotide sequence encodes a therapeutic protein or peptide. For example, the therapeutic nucleotide sequence may encode a wild-type (unmodified) protein to compensate for a modified (e.g., mutated or truncated) version of the protein present in a subject or to compensate for a protein the subject lacks. Non-limiting examples of proteins encoded by a therapeutic nucleotide sequence include antibodies, enzymes, hormones, growth factors, cytokines and fusion proteins.

In some embodiments, the therapeutic nucleotide sequence is configured to replace a disease allele. For example, the therapeutic nucleotide sequence could be designed to facilitate nonhomologous end joining or homologous recombination. In some embodiments, the therapeutic gene sequence is a programmable nuclease. Non limiting examples of programmable nucleases include Cas9, Cpf1, C2c2, zinc finger, zinc finger nucleases, TALEs, TALENS, meganucleases, and fusions thereof to effector domains. Effector domains include transcriptional activators, transcription repressors, transposes, recombinases and deaminases. In some embodiments, the therapeutic nucleotide sequence encodes a guide RNA (e.g., for gene editing) or a DNA template (e.g., for homologous recombination).

In some examples, the therapeutic nucleotide sequence itself is a therapeutic molecule. In some embodiments, the nucleotide sequence is a DNA aptamer that binds a molecular target (e.g., protein target). A process termed SELEX (systematic evolution of ligands by exponential enrichment) is frequently used to select oligonucleotides from a DNA library that bind strongly to a target (Zhou J et al. Ther Nucleic Acids. 2014;3:e169). Examples of DNA aptamers include AS1411, which binds to the cellular protein nucleolin and has been tested as an anticancer agent (Bates PJ et al. Exp Mol Pathol. 2009;86(3):151-64; Soundararajan S et al. Cancer Res. 2008;68(7):2358-65), and ARC1779, a PEGylated DNA aptamer which binds to von Willebrand factor to inhibit its interaction with platelets, thereby inducing an antithrombotic effect (Markus HS et al. Stroke. 2011;42(8):2149-53).

In certain embodiments, the therapeutic nucleotide sequence encodes a sequence that is capable of reducing expression of a disease gene. In some embodiments, the therapeutic nucleotide sequence is complementary to a mRNA encoding a disease gene. As a non-limiting example, the therapeutic nucleotide sequence may be a guide RNA (e.g., for use in CRISPR systems), a siRNA, a microRNA (miRNA), or a short hairpin RNA (shRNA). In some embodiments, a therapeutic nucleotide sequence targets a mutant allele.

In some embodiments, the recombinant viral genome comprises a promoter operably linked to the therapeutic nucleotide sequence. A promoter is a control region of a nucleic acid sequence at which initiation and rate of transcription of the remainder of a nucleic acid sequence are controlled. A promoter may also contain sub-regions at which regulatory proteins and molecules may bind, such as RNA polymerase and other transcription factors. A promoter drives expression or drives transcription of the nucleic acid sequence that it regulates. A promoter is considered to be ‘operably linked’ to a nucleotide sequence when it is in a correct functional location and orientation in relation to the nucleotide sequence to control (‘drive’) transcriptional initiation and/or expression of that sequence. Promoters may be constitutive or inducible. An inducible promoter is a promoter that is regulated (e.g., activated or inactivated) by the presence or absence of a particular factor.

In some instances, a suitable host cell line (e.g., HEK293T, HeLa cells and Sf9 insect cells) may be used for producing viral particles encoding the recombinant viral genomes disclosed herein following routine practice. One or more expression vectors (e.g. viral vectors) encoding viral components, at least one therapeutic nucleotide sequence and at least one inhibitory oligonucleotide described herein may be introduced into the suitable host cells, which can then be cultured under suitable conditions allowing for production of the viral particles. When needed, a helper virus can be used to facilitate replication and/or assembly of the viral particles. Alternatively, a host cell line producing one or more of essential viral components for viral genome replication and/or viral particle assembly may be used. The supernatant of the cell culture may be collected and the viral particles contained therein can be collected via routine methodology. As an example, a method for AAV production is provided in the Materials and Methods section below.

The recombinant viral genomes as provided herein may be administered by intravenous, intramuscular, subretinal, intravitreal, intrathecal, intraparenchymal, and intracranial injections. In some embodiments, the recombinant viral genomes is delivered by intramuscular injection. In some embodiments, the recombinant viral genomes is delivered by intravenous injection.

In some embodiments, the recombinant viral genomes are used to transduce cells in the liver, skeletal muscle, cardiac muscle, eye (e.g., retina), central nervous system or any combination thereof.

### Pharmaceutical Compositions

In some aspects, the present disclosure provides compositions comprising any of the recombinant viral genomes as disclosed herein. In some embodiments, the compositions further comprise a pharmaceutically-acceptable excipient. Non-limiting examples of pharmaceutically-acceptable excipients include water, saline, dextrose, glycerol, ethanol and combinations thereof. The excipient may be selected on the basis of the mode and route of administration, and standard pharmaceutical practice.

Recombinant viral genomes, in some embodiments, may be formulated in a delivery vehicle. Non-limiting examples of delivery vehicles include nanoparticles, such as nanocapsules and nanospheres. See, e.g., Sing, R et al. Exp Mol Pathol. 2009;86(3):215-223. A nanocapsule is often comprised of a polymeric shell encapsulating a drug (e.g., recombinant viral genome of the present disclosure). Nanospheres are often comprised of a solid polymeric matrix throughout which the drug (e.g. recombinant viral genome) is dispersed. In some embodiments, the nanoparticle is a lipid particle, such as a liposome. See, e.g., Puri, A et al. Crit Rev Ther Drug Carrier Syst. 2009;26(6):523-80. The term ‘nanoparticle’ also encompasses microparticles, such as microcapsules and microspheres.

Methods developed for making particles for delivery of encapsulated agents are described in the literature (for example, please see Doubrow, M., Ed., “Microcapsules and Nanoparticles in Medicine and Pharmacy,” CRC Press, Boca Raton, 1992; Mathiowitz and Langer. J. Controlled Release 5:13-22, 1987; Mathiowitz et al. Reactive Polymers 6:275-283,1987; Mathiowitz et al. J. Appl. Polymer Sci. 35:755-774, 1988; each of which is incorporated herein by reference).

General considerations in the formulation and/or manufacture of pharmaceutical agents, such as compositions comprising any of the recombinant viral genomes disclosed herein may be found, for example, in Remington's Pharmaceutical Sciences, 18th edition, Mack Publishing Co., Easton, Pa (1990) (incorporated herein by reference in its entirety).

### Methods of Delivery

Any of the recombinant viral genomes or compositions disclosed herein may be administered to a subject (e.g., mammalian subject, such as a human, mouse, rabbit, goat, sheep or pig) to inhibit the inflammatory response (e.g., inhibit induction of the inflammatory response). In some embodiments, the subject is in need of gene therapy. For example, the subject may have a genetic disorder (e.g., characterized by chromosomal abnormality and/or gene defects including mutation, truncation, insertion and deletion).

The subject may have, may be suspected of having, or may at risk for a disease. In some embodiments, the disease is an ocular disease. As used herein, an “ocular disease” or “eye disease” is a disease or condition of the eye (e.g., retinal disease). Non-limiting examples of conditions that affect the eye include Ectropion, Lagophthalmos, Blepharochalasis, Ptosis, Stye, Xanthelasma, Dermatitis, Demodex, leishmaniasis, loiasis, onchocerciasis, phthiriasis, (herpes simplex), leprosy, molluscum contagiosum, tuberculosis, yaws, zoster, impetigo, Dacryoadenitis, Epiphora, exophthalmos, Conjunctivitis, Scleritis, Keratitis, Corneal ulcer/Corneal abrasion, Snow blindness/Arc eye, Thygeson's superficial punctate keratopathy, Corneal neovascularization, Fuchs' dystrophy, Keratoconus, Keratoconjunctivitis sicca, Iritis, iris, Uveitis, Sympathetic ophthalmia, Cataract, Chorioretinal inflammation, Focal chorioretinal inflammation, chorioretinitis, choroiditis, retinitis, retinochoroiditis, Disseminated chorioretinal inflammation, exudative retinopathy, Posterior cyclitis, Pars planitis, chorioretinal inflammations, Harada's disease, Chorioretinal inflammation, choroid, Chorioretinal scars, Macula scars, posterior pole (postinflammatory) (post-traumatic), Solar retinopathy, Choroidal degeneration, Atrophy, Sclerosis, angioid streaks, choroidal dystrophy, Choroideremia, choroidal, areolar, (peripapillary), Gyrate atrophy, choroid, ornithinaemia, Choroidal haemorrhage, Choroidal haemorrhage, NOS (Not Otherwise Specified), Choroidal detachment, Chorioretinal, Chorioretinal inflammation, infectious and parasitic diseases, Chorioretinitis, syphilitic, toxoplasma, tuberculosis, chorioretinal, Retinal detachment, distorted vision, Retinoschisis, Hypertensive retinopathy, Diabetic retinopathy, Retinopathy, Retinopathy of prematurity, Age-related macular degeneration, macula, Macular degeneration, Bull's Eye Maculopathy, Epiretinal membrane, Peripheral retinal degeneration, Hereditary retinal dystrophy, Retinitis pigmentosa, Retinal haemorrhage, retinal layers, Central serous retinopathy, Retinal detachment, retinal disorders, Macular edema, macula, Retinal disorder, Diabetic retinopathy, Glaucoma, optic neuropathy, ocular hypertension, open-angle glaucoma, angle-closure glaucoma, Normal Tension glaucoma, open-angle glaucoma, angle-closure glaucoma, Floaters, Leber's hereditary optic neuropathy, Optic disc drusen, Strabismus, Ophthalmoparesis, eye muscles, Progressive external ophthaloplegia, Esotropia, Exotropia, Disorders of refraction, accommodation, Hypermetropia, Myopia, Astigmatism, Anisometropia, Presbyopia, ophthalmoplegia, Amblyopia, Leber's congenital amaurosis, Scotoma, Anopsia, Color blindness, Achromatopsia/Maskun, Nyctalopia, Blindness, River blindness, Micropthalmia/coloboma, Red eye, Argyll Robertson pupil, pupils, Keratomycosis, Xerophthalmia, and Aniridia.

In certain embodiments, the disease affects muscle. Non-limiting examples of muscle diseases include Barth syndrome, Duchenne muscular dystrophy. Becker muscular dystrophy, myotonic dystrophy, facioscapulohumeral muscular dystrophy, mitochondrial encephalomyopathy, MELAS syndrome, MERRF syndrome, MNGIE syndrome, mitochondrial myopathy, Kearns-Sayre syndrome, myalgia, fibromyalgia, polymyalgia rheumatica, myoma, myositis, dermatomyositis, neuromuscular disease, Kearns-Sayre syndrome, muscular dystrophy, myasthenia, congenital myasthenic syndrome, Lambert-Eaton myasthenic syndrome, myasthenia gravis, myotonia, myotonia congenita, spinal muscular atrophy, tetany, ophthalmoplegia, and rhabdomyolysis.

Suitable routes of administration include parenterally, by injection, for example, intravenously, subcutaneously, intramuscularly intrathecally, intraperitoneally, intraparenchymal, intracuteanously, intrasternally, intraarticularlly, intracranially, intralesionally, intrarectually, intravaginally, intranasally, intragastically, intratracheally, or intrapulmonarily. Alternatively, other modes of administration including suppositories, oral formulations, enteral, nasal, topical or transmucosal administration may be desirable. Oral formulations may include normally employed incipients such as, for example, pharmaceutical grades of saccharine, cellulose, magnesium carbonate and the like. These compositions may take the form of solutions, suspensions, tablets, pills, capsules, sustained release formulations or powders.

In some embodiments, a recombinant viral genome comprising an inhibitory oligonucleotide is administered intramuscularly. In some embodiments, the intramuscularly administered recombinant viral genome comprises an adeno-associated viral genome, which comprises a therapeutic nucleotide sequence and an inhibitory nucleotide sequence. In some embodiments, the inhibitory nucleotide sequence comprises a nucleotide sequence of any one of SEQ ID NOS: 1-7, 9-25, or 27-32. In some embodiments, the adeno-associated viral genome administered intramuscularly comprises a therapeutic nucleotide sequence and three tandem repeats of SEQ ID NO: 1. In some embodiments, the adeno-associated viral genome administered intramuscularly comprises a therapeutic nucleotide sequence and three tandem repeats of SEQ ID NO: 9. In some embodiments, the viral genome administered intramuscularly is expressed in muscle cells.

In some embodiments, the recombinant viral genomes of the present disclosure are administered intravenously to a subject. In some embodiments, the recombinant viral genomes are administered peritoneally to a subject. In some embodiments, the intravenously or peritoneally administered recombinant viral genome comprises an adeno-associated viral genome, which includes a therapeutic nucleotide sequence and an inhibitory nucleotide sequence. In some embodiments, the inhibitory nucleotide sequence comprises a nucleotide sequence of any one of SEQ ID NOS: 1-7, 9-25, or 27-32. In some embodiments, the adeno-associated viral genome administered intravenously or peritoneally comprises a therapeutic nucleotide sequence and three tandem repeats of SEQ ID NO: 1. In some embodiments, the adeno-associated viral genome administered intravenously or peritoneally comprises a therapeutic nucleotide sequence and three tandem repeats of SEQ ID NO: 9. In some embodiments, the recombinant viral genome administered intravenously or peritoneally is expressed in the liver cells of a subject.

An inflammatory response (e.g., local or systemic) may be assessed by measuring the level of cytokine activity and/or expression in a subject. In some embodiments, the level of expression and/or activity of IL-6, TNF, interferon (e.g., IFNα, IFNβ, and IFNγ), and/or CXCL10 is measured. Typically, the level of cytokine expression and/or activity correlates with the degree of the inflammatory response. Thus, a subject who has received a recombinant viral genome of the present disclosure (comprising a therapeutic nucleotide sequence and an inhibitory oligonucleotide) may have a reduction of or undetectable expression and/or activity levels of certain cytokines, indicative of a reduced or no inflammatory response, compared to a subject who has received a recombinant viral genome that does not include an inhibitory oligonucleotide. In some embodiments, the control inflammatory response for comparison is the inflammatory response elicited by a viral genome that does not comprise an inhibitory oligonucleotide as determined by the same or a substantially similar assay under the same or substantially similar conditions. Exemplary control viral genomes for adeno-associated virus include scAAV-eGFP-3xcontrol and scAAV-eGFP used in Examples 2, 3, 4 and 5 below.

In some embodiments, a recombinant viral genome of the present disclosure elicits an inflammatory response in the subject that is at least 2-fold lower than a control. For example, a recombinant viral genome may elicit an inflammatory response in the subject that is at least 3-fold, 4-fold, 5-fold, 6-fold, 7-fold, 8-fold, 9-fold, 10-fold, 25-fold or 50-fold lower than a control. In some embodiments, a recombinant viral genome elicits an inflammatory response in the subject that is at least 10% lower than a control. For example, a recombinant viral genome may elicit an inflammatory response in the subject that is at least 15%, 20%, 25%, 30%, 35%, 40%, 45%, 55%, 60%, 70%, 80% or 90% lower than a control. In some embodiments, a recombinant viral genome may inhibit induction of an inflammatory response compared to a control, such that the inflammatory response is undetectable. A control, in some embodiments, is an inflammatory response elicited in a subject by a viral genome that does not comprise an inhibitory oligonucleotide.

In certain embodiments, a recombinant viral genome of the present disclosure reduces AAV-induced pathology in the eye of a subject compared to control. A control, in some embodiments, is the pathology, including tissue damage and alteration in morphology, elicited in an organ by a viral genome that does not comprise an inhibitory oligonucleotide. For example, a recombinant viral genome of the present disclosure may reduce loss of cone outer segments, reduce shortening of cone outer segments, or alter the morphology of cone outer segments less than a control. For example, an eye receiving a recombinant viral genome of the present disclosure may have better preservation of cone outer segments and appeared morphologically closer to an eye receiving no viral genome.

Morphology of the eye or tissues of the eye can be determined using methods known in the art, including cone arrestin staining and opsin staining. Retinal images from in vivo optical coherence tomography (OCT) b-scans may be used to determine damage to outer retinal lamination. As a non-limiting example, the lengths of various types of damage (e.g., retinal detachment, non-severe laminar disruption, or severe laminar damage) on the optical coherence tomography (OCT) b-scans may be measured (see, e.g., Example 9). In some instances, a recombinant viral genome of the present disclosure elicits less or no severe laminar damage compared to a viral genome without an inhibitory oligonucleotide.

In some embodiments, a recombinant viral genome (and thus the therapeutic nucleotide sequence of the recombinant viral genome) of the present disclosure is expressed in cells of the subject at a level that is at least 2-fold greater than a control. For example, a recombinant viral genome may be expressed in cells of the subject at a level that is at least 3-fold, 4-fold, 5-fold, 6-fold, 7-fold, 8-fold, 9-fold, 10-fold, 25-fold or 50-fold higher than a control. In some embodiments, a recombinant viral genome of the present disclosure is expressed in cells of the subject at a level that is at least 10% higher than a control. For example, a recombinant viral genome may be expressed in cells of the subject at a level that is at least 15%, 20%, 25%, 30%, 35%, 40%, 45%, 55%, 60%, 70%, 80% or 90% higher than a control. A control, in some embodiments, is the expression level of a viral genome that does not comprise an inhibitory oligonucleotide.

In some embodiments, a therapeutically effective amount of a recombinant viral genome of the present disclosure is administered to a subject to treat a genetic disorder, such as a muscle disorder or a liver disorder. A therapeutically effective amount, in some embodiments, is an amount of a therapeutic nucleotide sequence (and/or a recombinant viral genomes) required to confer therapeutic effect on the subject. In some embodiments, a therapeutically effective amount is an amount of inhibitory oligonucleotide required to inhibit induction of an inflammatory response following administration of a recombinant viral genome (comprising a therapeutic nucleotide sequence and an inhibitory nucleotide sequence) of the present disclosure. Effective amounts vary, as recognized by those skilled in the art, depending on the route of administration, excipient usage, and co-usage with other active agents. Effective amounts depend on the subject to be treated, including, for example, the weight, sex and age of the subject as well as the strength of the subject's immune system and/or genetic predisposition. Suitable dosage ranges are readily determinable by one skilled in the art. The effective amount (and thus the dosage and/or dosing schedule) of the compositions disclosed herein may also depend on the type of the viral genome, the type of therapeutic nucleotide sequence, and/or the type of inhibitory oligonucleotide.

In some embodiments, the therapeutically effective amount of a recombinant viral genome of the present disclosure is at least 20% lower than the therapeutically effective amount of a viral genome not comprising an inhibitory oligonucleotide. For example, the therapeutically effective amount of a recombinant viral genome of the present disclosure may be at least at least 25%, 30%, 40%, 50% or 60% (but less than 100%) lower than the therapeutically effective amount of a viral genome not comprising an inhibitory oligonucleotide. In some embodiments, administration of a recombinant viral genome of the present disclosure at a reduced therapeutically effective amount results in expression of an encoded therapeutic molecule at a level that is equal to or greater than (e.g., at least 5%, 10%, 20%, 30%, 40%, or 50% greater than) expression of the same encoded therapeutic molecule from a viral genome not comprising an inhibitory oligonucleotide sequence.

### Additional Embodiments The present disclosure also provides the following additional embodiments encompassed by numbered paragraphs.

1. A method comprising administering to a subject a recombinant viral genome comprising a therapeutic nucleotide sequence and an inhibitory oligonucleotide that inhibits nucleic acid-sensing toll-like receptor (TLR) activation and/or signaling, wherein the recombinant viral genome inhibits inflammatory response to the recombinant viral genome in a target tissue.
2. The method of paragraph 1, wherein the target tissue is muscle.
3. The method of paragraph 1, wherein the target tissue is liver.
4. The method of paragraph 1, wherein the viral genome is adeno-associated virus (AAV).
5. The method of any one of paragraphs 1-4, wherein the TLR is TLR9.
6. The method of any one of paragraphs 1-5, wherein the inhibitory oligonucleotide binds to TLR9.
7. The method of any one of paragraphs 6, wherein the inhibitory oligonucleotide binds to TLR9 without activating TLR9 mediated signaling.
8. The method of paragraph 7, wherein the inflammatory nucleic acids comprise CpG oligodeoxynucleotides.
9. The method of any one of paragraphs 1-7, wherein the inhibitory oligonucleotide comprises a CCx(not-C)(not-C)xxGGG (SEQ ID NO: 63) motif, wherein x is any nucleic acid.
10. The method of any one of paragraphs 1-7, wherein the inhibitory oligonucleotide comprises a nucleotide sequence that is at least 90% identical to the nucleotide sequence of any one of SEQ ID NOS: 1-7, 9-25, or 27-32.
11. The method of paragraph 10, wherein the inhibitory oligonucleotide comprises a nucleotide sequence that is identical to the nucleotide sequence of any one of SEQ ID NOS: 1-7, 9-25, or 27-32.
12. The method of any one of paragraphs 1-7, wherein the inhibitory oligonucleotide comprises a TTAGGG motif.
13. The method of paragraph 12, wherein the inhibitory oligonucleotide comprises at least two tandem repeats of the TTAGGG motif.
14. The method of paragraph 13, wherein the inhibitory oligonucleotide comprises at least three tandem repeats of the TTAGGG motif.
15. The method of paragraph 14, wherein the inhibitory oligonucleotide comprises a nucleotide sequence that is identical to the nucleotide sequence identified by SEQ ID NO: 6 or SEQ ID NO: 9.
16. The method of paragraph 11, wherein the inhibitory oligonucleotide comprises a nucleotide sequence that is identical to the nucleotide sequence identified by SEQ ID NO: 1.
17. The method of paragraph 16, wherein the inhibitory oligonucleotide comprises multiple tandem repeats of the nucleotide sequence that is identical to the nucleotide sequence identified by SEQ ID NO: 1.
18. The method of any one of paragraphs 13-17, wherein the tandem repeat sequence are separated from each other by a polyA linker.
19. The method of any one of paragraphs 1-18, wherein the viral genome is from adeno-associated virus (AAV), adenovirus, herpes simplex virus, varicella, variola virus, hepatitis B, cytomegalovirus, JC polyomavirus, BK polyomavirus, monkeypox virus, Herpes Zoster, Epstein-Barr virus, human herpes virus 7, Kaposi's sarcoma-associated herpesvirus, or human parvovirus B19.
20. The method of paragraph 19, wherein the viral genome is an AAV genome.
21. The method of any one of paragraphs 1-20, wherein the inhibitory oligonucleotide is located in the 5′ untranslated region of the viral genome.
22. The method of any one of paragraphs 1-21, wherein the recombinant viral genome is expressed in cells of the subject at a level that is at least 2-fold greater than a control.
23. The method of paragraph 22, wherein the recombinant viral genome is expressed in cells of the subject at a level that is at least 5-fold greater than a control.
24. The method of paragraph 23, wherein the recombinant viral genome is expressed in cells of the subject at a level that is at least 10-fold greater than a control.
25. The method of paragraph 24, wherein the recombinant viral genome is expressed in cells of the subject at a level that is at least 15-fold greater than a control.
26. The method of any one of paragraphs 1-25, wherein administration of the recombinant viral genome elicits an inflammatory response in the subject that is at least 2-fold lower than a control.
27. The method of paragraph 26, wherein administration of the recombinant viral genome elicits an inflammatory response in the subject that is at least 5-fold lower than a control.
28. The method of paragraph 27, wherein administration of the recombinant viral genome elicits an inflammatory response in the subject that is at least 10-fold lower than a control.
29. The method of paragraph 28, wherein administration of the recombinant viral genome elicits an inflammatory response in the subject that is at least 50-fold lower than a control.
30. The method of any one of paragraphs 22-29, wherein the control is expression of a viral genome that does not comprise the inhibitory oligonucleotide.
31. The method of any one of paragraphs 1-30, wherein the recombinant viral genome is administered intramuscularly.
32. The method of paragraph 31, wherein the recombinant viral genome is expressed in muscle cells of the subject.
33. The method of any one of paragraphs 1-30, wherein the recombinant viral genome is administered intravenously.
34. The method of paragraph 33, wherein the recombinant viral genome is expressed in liver cells of the subject.
35. The method of any one of paragraphs 1-34, wherein the subject is in need of gene therapy.
36. The method of any one of paragraphs 1-35, wherein the therapeutic nucleotide sequence encodes a therapeutic molecule.
37. The method of paragraph 36, wherein the therapeutic molecule is a therapeutic RNA or therapeutic DNA.
38. The method of paragraph 36, wherein the therapeutic molecule is a therapeutic protein or peptide.
39. The method of paragraph 37 or 38, wherein a therapeutically effective amount of the recombinant viral genomes is administered to the subject to treat a genetic disorder.
40. The method of paragraph 39, wherein the therapeutically effective amount is reduced by at least 20% relative to the therapeutically effective amount of a control, wherein the control is a recombinant viral genome that does not comprise an inhibitory oligonucleotide that inhibits nucleic acid-sensing TLR activation and/or signaling.
41. The method of paragraphs 40, wherein the therapeutic molecule is expressed in cells of the subject at a level that is equal to or greater than the expression level of a control, wherein the control is the therapeutic molecule encoded by a viral genome that does not comprise an an inhibitory oligonucleotide that inhibits nucleic acid-sensing TLR activation and/or signaling.
42. A recombinant viral genome comprising a therapeutic nucleotide sequence and an inhibitory oligonucleotide that inhibits nucleic acid-sensing toll-like receptor (TLR) activation and/or signaling.
43. The recombinant viral genome of paragraph 42, wherein the TLR is TLR3, TLR7, TLR8 or TLR9.
44. The recombinant viral genome of paragraph 43, wherein the TLR is TLR9.
45. The recombinant viral genome of any one of paragraphs 42-44, wherein the inhibitory oligonucleotide binds to the TLR.
46. The recombinant viral genome of any one of paragraphs 42-45, wherein the inhibitory oligonucleotide binds to inflammatory nucleic acids.
47. The recombinant viral genome of paragraph 46, wherein the inflammatory nucleic acids comprise CpG oligodeoxynucleotides.
48. The recombinant viral genome of any one of paragraphs 42-47, wherein the inhibitory oligonucleotide comprises a CCx(not-C)(not-C)xxGGG (SEQ ID NO: 63) motif, wherein x is any nucleic acid.
49. The recombinant viral genome of any one of paragraphs 42-48, wherein the inhibitory oligonucleotide comprises a nucleotide sequence that is at least 90% identical to the nucleotide sequence of any one of SEQ ID NOS: 1-7, 9-25, or 27-32.
50. The recombinant viral genome of paragraph 9, wherein the inhibitory oligonucleotide comprises a nucleotide sequence that is identical to the nucleotide sequence of any one of SEQ ID NOS: 1-7, 9-25, or 27-32.
51. The recombinant viral genome of any one of paragraphs 42-48, wherein the inhibitory oligonucleotide comprises a TTAGGG motif.
52. The recombinant viral genome of paragraph 51, wherein the inhibitory oligonucleotide comprises at least two tandem repeats of the TTAGGG motif.
53. The recombinant viral genome of paragraph 52, wherein the inhibitory oligonucleotide comprises at least three tandem repeats of the TTAGGG motif.
54. The recombinant viral genome of paragraph 53, wherein the inhibitory oligonucleotide comprises a nucleotide sequence that is identical to the nucleotide sequence identified by SEQ ID NO: 6 or SEQ ID NO: 9.
55. The recombinant viral genome of paragraph 50, wherein the inhibitory oligonucleotide comprises a nucleotide sequence that is identical to the nucleotide sequence identified by SEQ ID NO: 1.
56. The recombinant viral genome of paragraph 55, wherein the inhibitory oligonucleotide comprises multiple tandem repeats of the nucleotide sequence that is identical to the nucleotide sequence identified by SEQ ID NO: 1.
57. The recombinant viral genome of any one of paragraphs 52-56, wherein the tandem repeat sequence are separated from each other by a polyA linker.
58. The recombinant viral genome of any one of paragraphs 42-57, wherein the viral genome is from adeno-associated virus (AAV), adenovirus, herpes simplex virus, varicella, variola virus, hepatitis B, cytomegalovirus, JC polyomavirus, BK polyomavirus, monkeypox virus, Herpes Zoster, Epstein-Barr virus, human herpes virus 7, Kaposi's sarcoma-associated herpesvirus, or human parvovirus B19.
59. The recombinant viral genome of paragraph 58, wherein the viral genome is an AAV genome.
60. The recombinant viral genome of any one of paragraphs 42-59, wherein the inhibitory oligonucleotide is located in the 3′ untranslated region of the viral genome.
61. The recombinant viral genome of any one of paragraphs 42-60, wherein the therapeutic nucleotide sequence encodes a therapeutic molecule.
62. The recombinant viral genome of paragraph 61, wherein the therapeutic molecule is a therapeutic RNA.
63. The recombinant viral genome of paragraph 61, wherein the therapeutic molecule is a therapeutic protein or peptide.
64. A method of treating a condition in a subject comprising administering by intramuscular injection or intravenous injection to the subject a therapeutically effective amount of a recombinant adeno-associated viral genome comprising a therapeutic nucleotide sequence and an inhibitory oligonucleotide that comprises a nucleotide sequence that is identical to the nucleotide sequence identified by SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 9, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23 or SEQ ID NO: 24, to treat the condition in the subject without eliciting an inflammatory response.
65. A recombinant adeno-associated viral genome comprising a therapeutic nucleotide sequence and an inhibitory oligonucleotide that comprises a nucleotide sequence that is identical to the nucleotide sequence of any one of SEQ ID NOS: 1-7, 9-25, or 27-32.
66. A method comprising administering to a subject a recombinant viral genome comprising a therapeutic nucleic acid and an inhibitory oligonucleotide that inhibits the production of inflammatory cytokines.
67. A recombinant viral genome comprising a therapeutic nucleic acid and an inhibitory oligonucleotide that inhibits the production of inflammatory cytokines.
68. The recombinant viral genome of any one of paragraphs 42-59, wherein the inhibitory oligonucleotide is located in the 3′ untranslated region of the viral genome.
69. The method of paragraph 37, wherein therapeutic molecule is a therapeutic DNA.
70. The method of paragraph 69. wherein the therapeutic DNA is an aptamer.
71. The method of any one of paragraphs 1-41, wherein the recombinant viral genome further comprises at least one other inhibitory oligonucleotide that inhibits nucleic acid-sensing TLR activation and/or signaling.
72. The method of paragraph 71, wherein one of the inhibitory oligonucleotides is oriented in the sense direction of the viral genome and another of the inhibitory oligonucleotides is oriented in the antisense direction of the viral genome.
73. The method of paragraph 71 or 72, wherein each of the inhibitory oligonucleotides comprises a nucleotide sequence that is at least 90% identical to the nucleotide sequence of any one of SEQ ID NOS: 1-7, 9-25, or 27-32.
74. The method of any one of paragraphs 71-73, wherein one of the inhibitory oligonucleotides comprises SEQ ID NO: 9.
75. The method of any one of paragraphs 71-74, wherein one of the inhibitory oligonucleotides comprises SEQ ID NO: 5.
76. The recombinant viral genome of any one of paragraphs 42-63, wherein the recombinant viral genome further comprises at least one other inhibitory oligonucleotide that inhibits nucleic acid-sensing TLR activation and/or signaling.
77. The recombinant viral genome of paragraph 76, wherein one of the inhibitory oligonucleotides is oriented in the sense direction of the viral genome and another of the inhibitory oligonucleotides is oriented in the antisense direction of the viral genome.
78. The recombinant viral genome of paragraph 76 or 77, wherein each of the inhibitory oligonucleotides comprises a nucleotide sequence that is at least 90% identical to the nucleotide sequence of any one of SEQ ID NOS: 1-7. 9-25, or 27-32.
79. The recombinant viral genome of any one of paragraphs 76-78, wherein one of the inhibitory oligonucleotides comprises SEQ ID NO: 9.
80. The recombinant viral genome of any one of paragraphs 76-80, wherein one of the inhibitory oligonucleotides comprises SEQ ID NO: 5.
81. A method comprising administering to an eye of a subject a recombinant viral genome comprising a therapeutic nucleotide sequence and an inhibitory oligonucleotide that inhibits nucleic acid-sensing toll-like receptor (TLR) activation and/or signaling, wherein the recombinant viral genome inhibits an inflammatory response to the recombinant viral genome in the eye, optionally wherein the recombinant viral genome is administered intravitreally.

### EXAMPLES

### Example 1: Impact of Oligonucleotides on TLR9 Activation in Vitro

This example provides data indicating that incorporating TLR9 inhibitory oligonucleotides into DNA can reduce TLR9-mediated inflammation. Inhibitory oligonucleotides were covalently linked to a short nucleic acid that normally activates TLR signaling to determine whether the inhibitory oligonucleotides would block TLR9 activation by the short nucleic acid. The inhibitory oligonucleotides tested included: c41 oligonucleotide (SEQ ID NO: 1), ODN 2088 (SEQ ID NO: 2), ODN 4084-F (SEQ ID NO: 3), ODN INH-1 (SEQ ID NO: 4), ODN INH-18 (SEQ ID NO: 5), ODN TTAGGG (SEQ ID NO: 6), G-ODN (SEQ ID NO: 7), ODN 2114 (SEQ ID NO: 16), ODN 4024 (SEQ ID NO: 17), ODN INH-4 (SEQ ID NO: 18), ODN INH-13 (SEQ ID NO: 19), ODN Poly-G (SEQ ID NO: 20), ODN GpG (SEQ ID NO: 21), ODN IRS-869 (SEQ ID NO: 22), ODN IRS-954 (SEQ ID NO: 23) and ODN 21158 (SEQ ID NO: 24). ODN Control (15 nt) and ODN Control (24 nt) were used as controls.

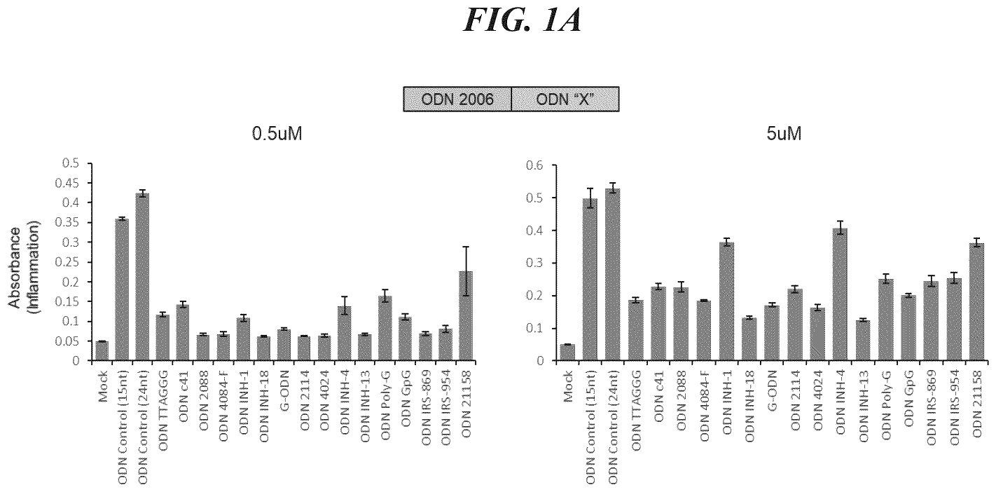

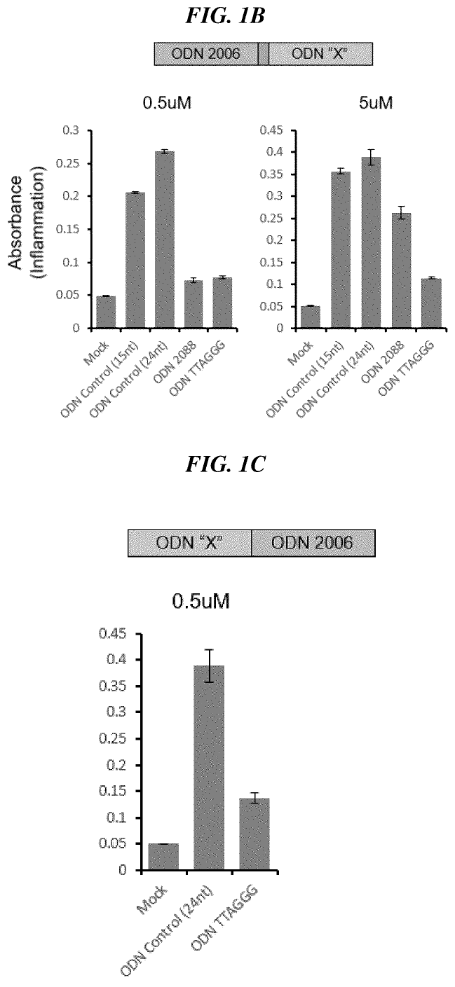

Several single-stranded DNA oligonucleotides were generated in which ODN 2006, a CpG-containing oligonucleotide known to strongly activate TLR9, is immediately followed by a control or experimental oligonucleotide (i.e. ODN 2006 is on the 5′ end). All oligonucleotides were synthesized with a phosphorothioate backbone for increased stability. A HEK293-based reporter cell line that constitutively expresses TLR9 to measure TLR9-mediated inflammation was used. When oligonucleotides were applied at the low concentration of 0.5 μM, both oligonucleotides containing control sequences induced robust inflammation compared to mock treatment (FIG. 1A on the left). In contrast, all the oligonucleotides containing TLR9 inhibitory oligonucleotides showed markedly reduced inflammation (FIG. 1A on the left). A similar trend at the high concentration of 5 μM was observed (FIG. 1A on the right).

Oligonucleotides with an AAAAA linker (SEQ ID NO: 8) between ODN 2006 and the following sequence were also tested, and similar results were observed with reduction in inflammation (FIG. 1B), suggesting the TLR9 inhibitory sequence can be distal to the inflammatory sequence.

When oligonucleotides in which the order of ODN 2006 and the covalently linked sequence is reversed were tested, the oligonucleotide containing TLR9 inhibitory oligonucleotide ODN TTAGGG was also able to reduce inflammation (FIG. 1C). Taken together, these results show that incorporation of a TLR9 inhibitory oligonucleotide in a DNA inhibits inflammatory responses.

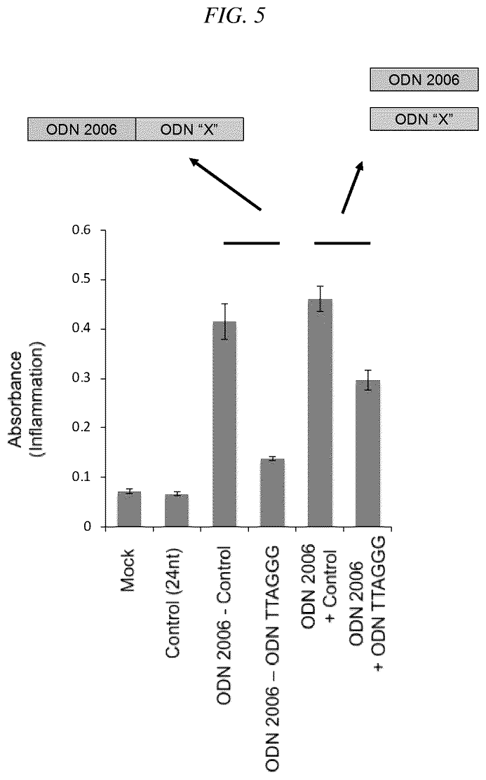

A condition was identified where ODN 2006 covalently linked to a control sequence (in cis) gave comparable inflammation as ODN 2006 and the control ODN co-administered at the same concentration (in trans), and observed that ODN 2006-TTAGGG (fusion of SEQ ID NOS: 26 and SEQ ID NO: 6) blocked ˜80% of induced inflammation while co-administration of ODN 2006 (SEQ ID NO: 26) and ODN TTAGGG (SEQ ID NO: 6) only inhibited ˜35% of induced inflammation (FIG. 5). Thus, linking a TLR9 inhibitory sequence to an otherwise inflammatory nucleic acid (in cis), for example, can be more effective at preventing inflammation than administering the TLR9 inhibitory sequence as an independent molecule (in trans).

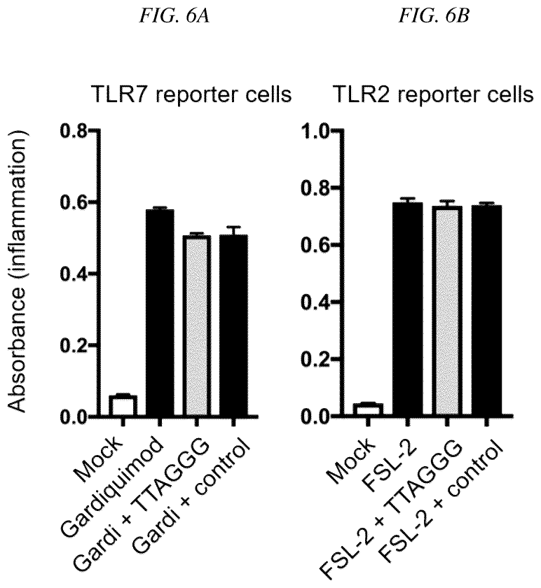

ODN TTAGGG did not reduce inflammatory responses by TLR7 stimulation (FIG. 6A) or TLR2 stimulation (FIG. 6B) even at high concentration of 5 μM, supporting its specificity for TLR9.

### Example 2: Engineering a Self-Complementary AAV Vector

It is unknown if the inhibitory oligonucleotides retain functionality in the context of a much larger viral genome (i.e., the sequence is covalently linked on both ends to much longer sequences). To test this possibility, a self-complementary (sc) AAV vector encoding enhanced green fluorescent protein (eGFP) was used, and 3 copies of c41 oligonucleotide (3×SEQ ID NO: 1) or telomere (SEQ ID NO: 9), derived from bacteria and mammalian telomeres respectively (Gursel, I et al. J Immunol. 2003;171(3):1393-400; Kaminski, JJ et al. J Immunol. 2013;191(7):3876-83; Shirota, H et al. J Immunol. 2005;174(8):4579-83; Li, Y et al. Vaccine. 2011;29(11):2193-8), were inserted into a plasmid harboring the vector genome. sc AAV vectors were used as they have been shown to be more efficient at triggering TLR9 activation and inducing more inflammation in the mouse liver than single-stranded (ss) AAV vectors. As c41 oligonucleotide and telomere oligonucleotides are predicted to have strong secondary structure, an AAAAA (SEQ ID NO: 8) linker was inserted between copies of the inhibitory oligonucleotide. In addition, 3xc41 and 3x telomere sequences were placed after the polyA sequence and upstream of the right inverted terminal repeat (ITR) so they would be present in the DNA genome during viral entry, but would be absent from subsequent mRNA transcripts upon successful transduction (scAAV-eGFP-3xc41 and scAAV-eGFP-3x telomere). Finally, to determine if the location of inhibitory oligonucleotide in the viral genome matters, a vector where 3x telomere was located between the left ITR and the promoter was generated (scAAV-3x telomere-eGFP) (data not shown).

Example 3: Impact of Three Copies of Telomere Oligonucleotide (“3xtelomere” SEQ ID NO: 35) on the Inflammatory Response Elicited by a Self-Complementary Adeno-Associated Virus (scAAV) Genome and on Transgene Expression in Muscle Tissue of Mice in Vivo

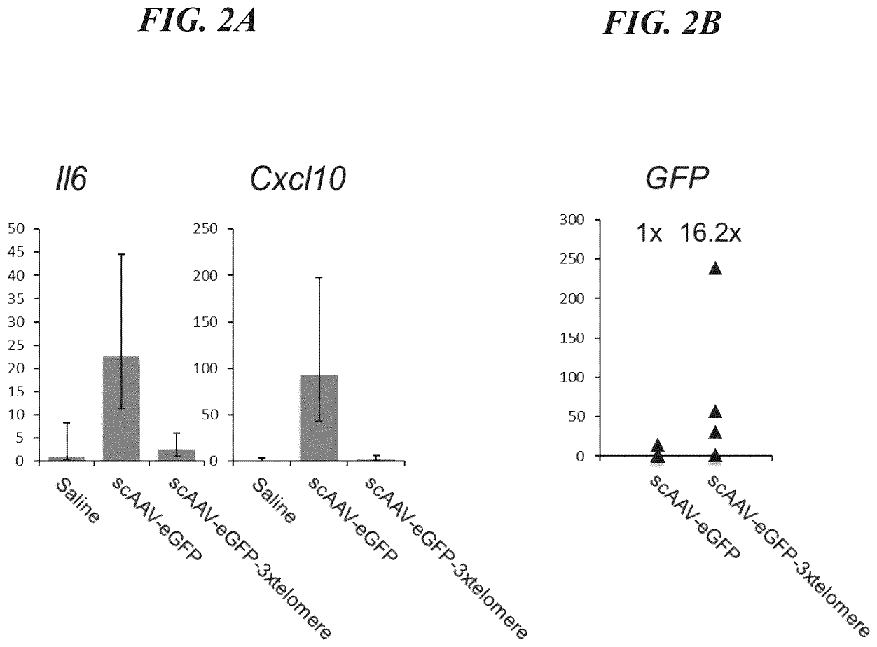

Muscle is an important tissue target for gene therapy. The scAAV-eGFP-3x telomere was selected for in vivo characterization as the telomere oligonucleotide is derived from human sequences and might be preferable for clinical use. To determine whether three copies of the telomere oligonucleotide (“3xtelomere” SEQ ID NO: 35) could prevent an inflammatory response induced by AAV, the following was delivered to the quadriceps of adult C57BL/6 mice via intramuscular injections: control saline, scAAV-eGFP or scAAV-eGFP-3xtelomere (described in example 2). Consistent with the literature, scAAV-eGFP increased Il6 and Cxcl10 expression in muscle tissues 2 h post-administration (approximately 20 to 100 fold, compared to saline), indicating inflammation (FIG. 2A). In contrast, scAAV-eGFP-3x telomere showed little to no increase in inflammatory markers (FIG. 2A). Furthermore, 28 d after AAV administration, scAAV-eGFP-3x telomere showed 16.2×higher gene expression of GFP than scAAV-eGFP. demonstrating increased transgene expression (FIG. 2B). Therefore, when linked to an AAV-encoding a transgene, the telomere oligonucleotide could also increase viral genome expression.

### Example 4: Impact of Telomere and c41 Oligonucleotides on Inflammatory Response Elicited by a Self-Complementary Adeno-Associated Virus (scAAV) Genome in Liver Tissue of Mice in Vivo

Intravenous delivery of AAV is often used to transduce hepatocytes for gene therapy. Previous work has shown that upon intravenous administration of AAV, Kupffer cells (resident hepatic antigen-presenting cells) in the liver of mice are capable of sensing sc AAV genomes and triggering inflammatory and innate immune responses 1-9 h later (Martino, AT et al., Blood. 2011;117(24):6459-68). These responses include induction of proinflammatory cytokines such as TNF and IL6. In addition, immune cells such as neutrophils, macrophages and natural killer (NK) cells infiltrate the liver 2 h after AAV administration.

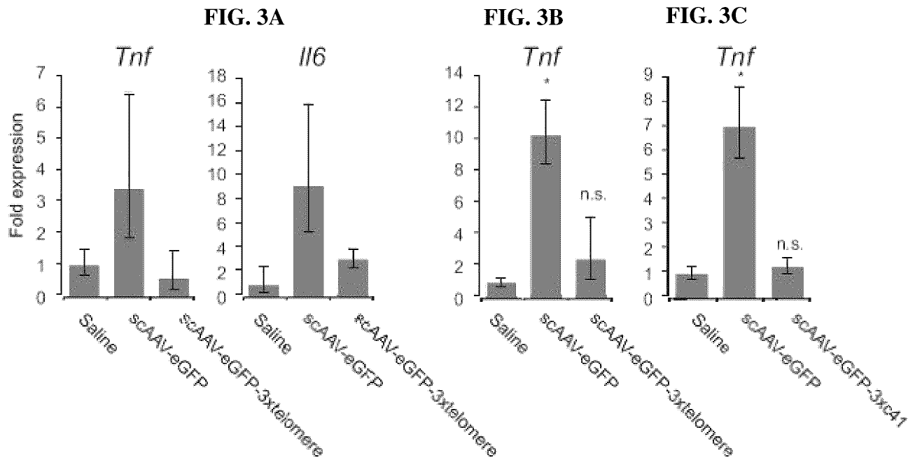

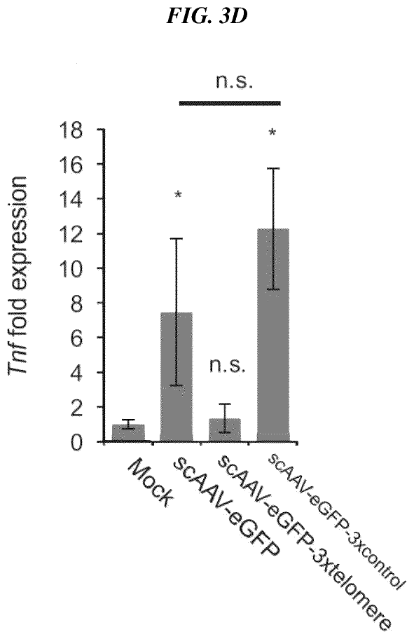

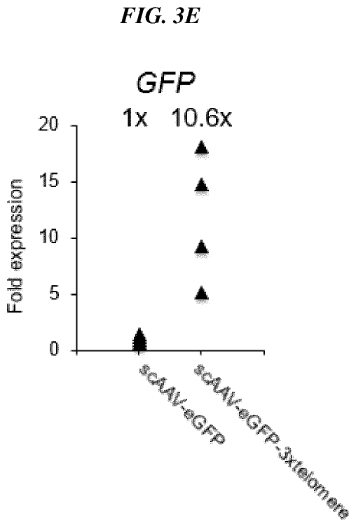

To determine if the engineered vectors can reduce inflammation in the liver in vivo, PBS or equal amounts of scAAV-eGFP or scAAV-eGFP-3x telomere was administered via tail vein injection. In agreement with previous work, scAAV-eGFP stimulated increased Tnf and Il6 expression in the liver (approximately 3 to 10 fold, compared to saline), indicating inflammation (FIG. 3A). In contrast, scAAV-eGFP-3x telomere showed little to no increase in inflammatory markers (FIG. 3A). More mice were tested in subsequent experiments and scAAV-eGFP stimulated statistically significant Tnf induction in the liver compared to saline, while scAAV-eGFP-3x telomere and scAAV-eGFP-3xc41 did not (FIGS. 3B-3C), demonstrating their ability to evade eliciting inflammation in the liver. Finally, it was determined that scAAV-eGFP-3xcontrol is not able to prevent inflammation in the liver compared to scAAV-eGFP, demonstrating that the inhibitory oligonucleotide sequence plays a role in preventing inflammation (FIG. 3D).

### Example 5: Impact of Three Copies of Telomere Oligonucleotide on Transgene Expression by a scAAV in Vivo in Liver Tissue

To determine if the engineered vectors can also increase transgene expression in the liver in vivo, scAAV-eGFP or scAAV-eGFP-3x telomere was administered to mice via intravenous injection and GFP gene expression was measured in the liver 14 d later. 10.6×higher gene expression of GFP by scAAV-eGFP-3x telomere was observed (FIG. 3E), suggesting that the engineered vectors also increase transgene expression in the liver.

Example 6: Engineering a Self-Complementary AAV Vector Encoding Human Factor IX (FIX) and the Impact of Three Copies of Telomere Oligonucleotide on Innate Immune Response and Transgene Expression in Vivo in Liver Tissue

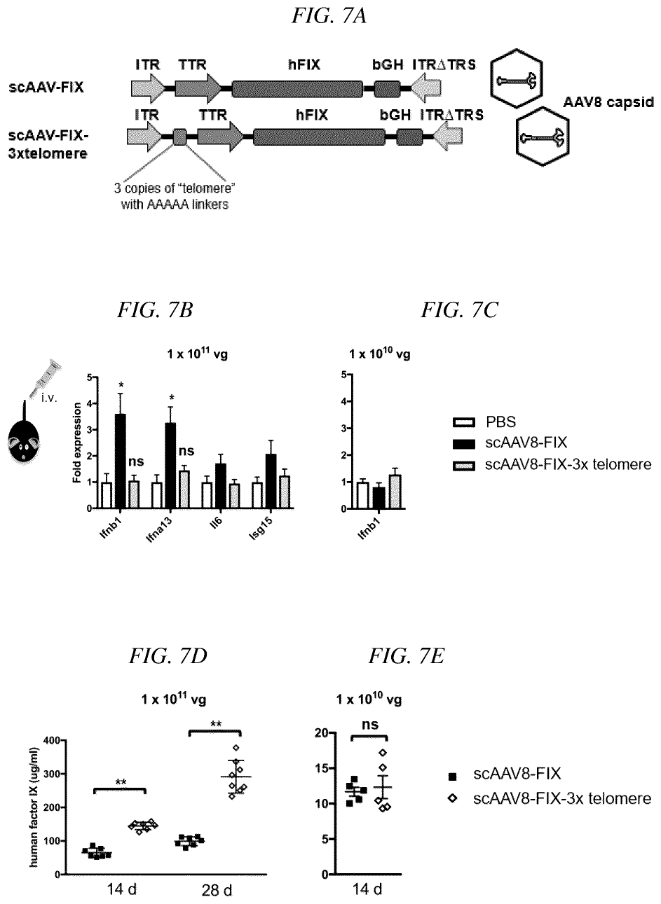

Three copies of SEQ ID NO: 9 were inserted into a plasmid harboring a vector genome for a self-complementary AAV vector expressing human FIX (FIG. 7A). An AAAAA (SEQ ID NO: 8) linker was inserted between copies of the inhibitory oligonucleotide (FIG. 7A). The 3x telomere sequences were placed after the left inverted terminal repeat (ITR) but before the transthyretin (TTR) promoter so they would be present in the DNA genome during viral entry. but would be absent from subsequent mRNA transcripts upon successful transduction. To determine if the engineered vectors can reduce inflammation in the liver in vivo, PBS or equal amounts of scAAV-FIX or scAAV-FIX-3x telomere (1×10¹¹ vg of each, or 1×10¹⁰ vg of each, in an AAV8 capsid) was administered via tail vein injection. In agreement with previous work, scAAV-FIX stimulated increased interferon (Ifnb1 and Ifna13) gene expression in the liver compared to saline 2 h after administration, indicating innate immune responses (FIG. 7B). In contrast, scAAV-FIX-3x telomere showed little to no increase in interferon expression (FIG. 7B). Furthermore, scAAV-FIX-3x telomere-treated mice expressed more human FIX protein in plasma compared to scAAV-FIX treatment 14 d and 28 d later (FIG. 7D). A 10-fold lower dose of AAV vectors did not result in interferon responses (FIG. 7C) or differences in human FIX protein expression in plasma (FIG. 7E), suggesting that the two vectors do not have inherently different potencies and that evading interferon responses enhances transgene expression.

### Example 7: Intramuscular Injection of Engineered Single-Stranded AAV Vector in Vivo in Mice

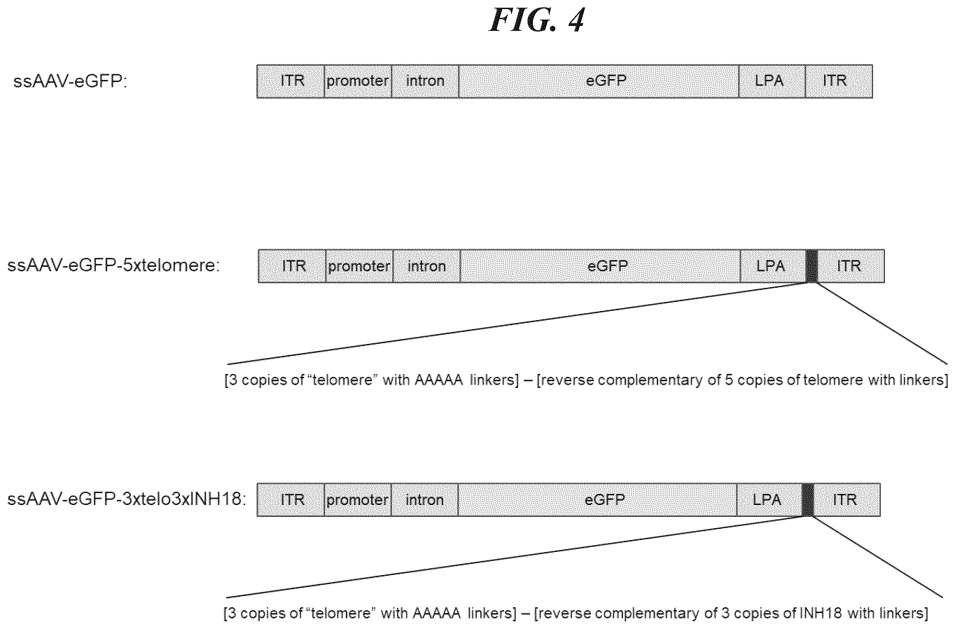

SSAAV-eGFP and ssAAV-eGFP-3xtelo3xINH18 (FIG. 4) were packaged in an AAVrh32.33 capsid and intramuscularly injected into the quadriceps muscle of mice. While dose-dependent immune responses are well-appreciated in clinical use, robust immune responses are generally not observed in mice upon AAV administration, especially ss AAV vectors which are more widely used and have a larger coding capacity. Thus, the more clinically relevant immunogenic condition was modeled in mice by utilizing AAVrh32.33 capsid (hereafter referred to as rh32.33) and intramuscular delivery. This combination has been shown to lead to robust CD8+ T cell responses against rh32.33 capsid, local infiltration of cytotoxic T cells into the muscle, and a decline in transgene expression over time in wild-type mice (Faust et al., J Clin Invest 123, 2994-3001 (2013); Mays et al., J Immunol 182, 6051-6060 (2009). Furthermore, the authors found that Tlr9^{-/-} mice showed substantially reduced T cell responses against rh32.33 capsid and maintained stable transgene expression, and similar benefits were observed in wild-type mice by partially depleting the AAV vector genome of CpG motifs (Faust et al., J Clin Invest 123, 2994-3001 (2013)).

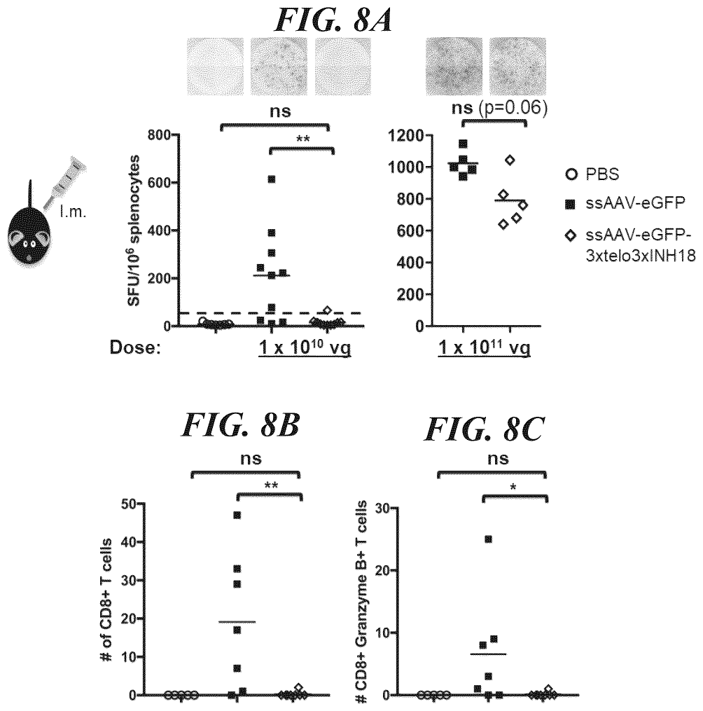

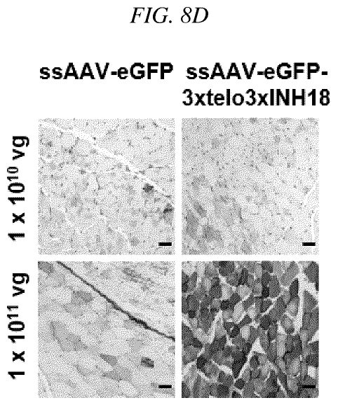

At a lower dose of 1×10¹⁰ vg, ssAAV-eGFP triggered a range of CD8+ T cell responses against rh32.33 capsid, with 7 of 10 animals showing positive T cell reactivity (FIG. 8A). In contrast, ssAAV-eGFP-3xtelo3xINH18, which harbors the inhibitory oligonucleotide designed for single-stranded vectors, showed nearly no CD8+ T cell response against rh32.33 capsid (1 of 10 animals positive) and was not statistically different from PBS treatment (FIG. 8A). At a higher dose of 1×10¹¹ vg, both vectors elicited extremely high capsid-directed T cell responses (˜800-1000 SFU/million splenocytes); while not statistically significant, the engineered vector elicited a modestly weaker T cell capsid response than the parental vector, consistent with the idea that the inhibitory oligonucleotide functions in an AAV dose-dependent manner (FIG. 8A). Cytotoxic T cell infiltrates have been observed in muscle biopsies of patients receiving intramuscular AAV gene therapy (Ferreira et al., Front Immunol 5, 82 (2014); Flotte et al., Hum Gene Ther 22, 1239-1247 (2011)). Thus, immune cell infiltration into the local tissue environment was further characterized. RobustCD8+ T cell infiltration was observed into muscle samples from ssAAV-eGFP-injected animals, and approximately one-third were granzyme B+, a maker for activated cytotoxic T cells, while no CD8+ T cell infiltration was observed in all eight ssAAV-eGFP-3xtelo3xINH18-injected muscle samples, strongly suggesting that the presence of the inhibitory oligonucleotide prevented capsid-directed T cell responses and infiltration (FIG. 8B-8C). The engineered vector showed comparable or higher GFP expression than the parental vector by immunohistochemical analysis (FIG. 8D).

### Example 8: Engineering a Single-Stranded AAV Vector

A single-stranded AAV vector, ssAAV-eGFP, was modified by inserting 5 copies of the telomere sequence (5×SEQ ID NO: 9) with an AAAAA (SEQ ID NO: 8) linker between each copy, followed by another 5 copies of the telomere sequence (5×SEQ ID NO: 9) in anti-sense direction with a linker between each copy, giving ssAAV-eGFP-5xtelomere (FIG. 4). Since both positive and negative strands of the viral genome are equally likely to be packaged into a viral particle, this ensures that each packaged viral genome would have 5 copies of the telomere sequence (5×SEQ ID NO: 9) in the correct orientation. ssAAV-eGFP-3xtelo3xINH18, which carries 3 copies of the telomere sequence (3×SEQ ID NO: 9) with AAAAA (SEQ ID NO: 8) linkers, followed by 3 copies of INH-18 (3×SEQ ID NO: 5) with linkers but in anti-sense direction was also engineered (FIG. 4). INH-18 (SEQ ID NO: 5) was chosen as it showed potent inhibition of TLR9-mediated inflammation in the oligonucleotide assays described above (FIG. 1A on the right). Furthermore, having 3 copies of the telomere sequence (3×SEQ ID NO: 9) in the sense direction followed by 3 copies of INH-18 (3×SEQ ID NO: 5) in the anti-sense direction avoids the possibility of self-annealing via complementarity, which may hamper viral packaging. Finally, since both positive and negative strands of the viral genome are equally likely to be packaged into a viral particle, this design increases the probability that each packaged viral genome will have either 3 copies of the telomere sequence (3×SEQ ID NO: 9) in the correct orientation or 3 copies of INH-18 (3×SEQ ID NO: 5) in the correct orientation.

The three vectors were produced as purified AAV8 viruses and total yield of viruses was measured via PCR of viral genomes. ssAAV-eGFP and ssAAV-eGFP-3xtelo3xINH18 gave similar yields of 3.97×10¹³ vg and 3.60×10¹³ vg, while ssAAV-eGFP-5xtelomere gave ˜10-fold lower yield of 3.11×10¹⁷ vg (Table 1). Table 1 includes yields of indicated AAV8 viruses produced after triple transfection and purification and yield is shown as total amount of viral genomes (vg) obtained for each vector. Thus, ssAAV-eGFP-5xtelomere may have packaging issues and results in lower viral yields compared to the parental ssAAV-eGFP vector, while ssAAV-eGFP-3xtelo3xINH18 gives comparable viral yields.

TABLE 1

| Name of virus | Total yield (vg) |
|---|---|
| ssAAV-eGFP | 3.97 × 10¹³ |
| ssAAV-eGFP-5xtelomere | 3.11 × 10¹² |
| ssAAV-eGFP-3xtelo3xINH18: | 3.60 × 10¹³ |

### Example 9: Subretinal Injection of Engineered Single-Stranded AAV Vector in Vivo in Outbred Pigs

The eye is often described as “immune-privileged” due in part to the presence of a blood-retina barrier that limits the entry of immune cells and immune factors. However, innate and adaptive immune responses have been reported in both large animal studies and clinical trials following subretinal AAV administration with dose-dependent severity (Bainbridge et al., N Engl J Med 372, 1887-1897 (2015); Ramachandran et al., Hum Gene Ther 28, 154-167 (2017); Reichel et al., Mol Ther 25, 2648-2660 (2017)). To determine immune responses and pathology in the retina with AAV, a subretinal AAV pig model was used as the pig eye shares a similar size and morphology as the human eye (Sanchez et al., Graefes Arch Clin Exp Ophthalmol 249, 475-482 (2011)). Table 2 shows design of study evaluating subretinal delivery of AAV vectors in wild-type pigs. Six wild-type female pigs were injected subretinally with 75 μl of indicated AAV8 vectors (dose of 4×10¹¹ vg per eye) or with vehicle control. The animals received clinical examinations at weekly intervals and OCT imaging at 2 wpi and 6 wpi, and were euthanized 6 wpi, wpi, weeks post-injection: OD, oculus dextrus (right eye): OS, oculus sinister (left eye): N.A., not applicable.

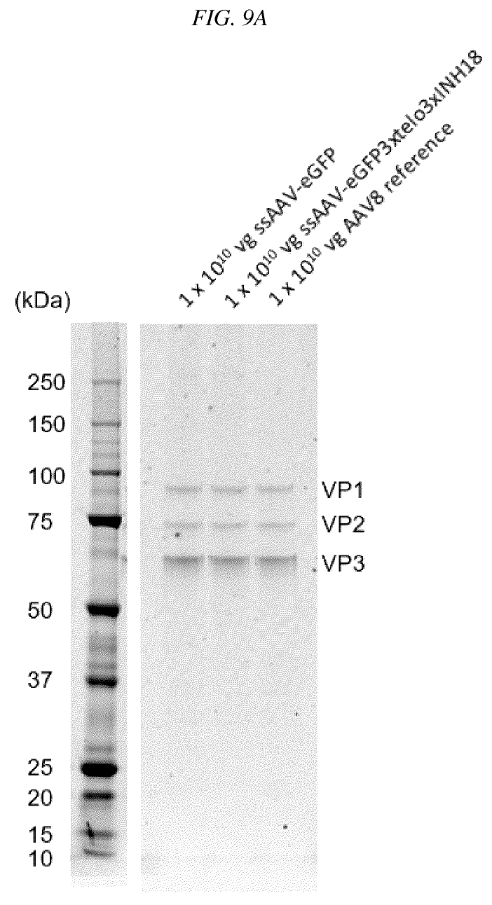

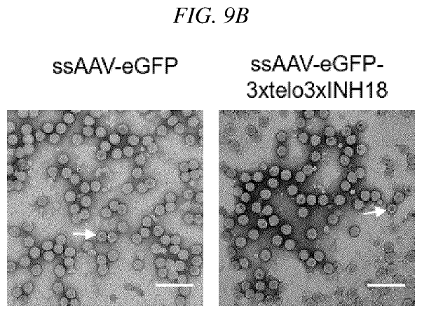

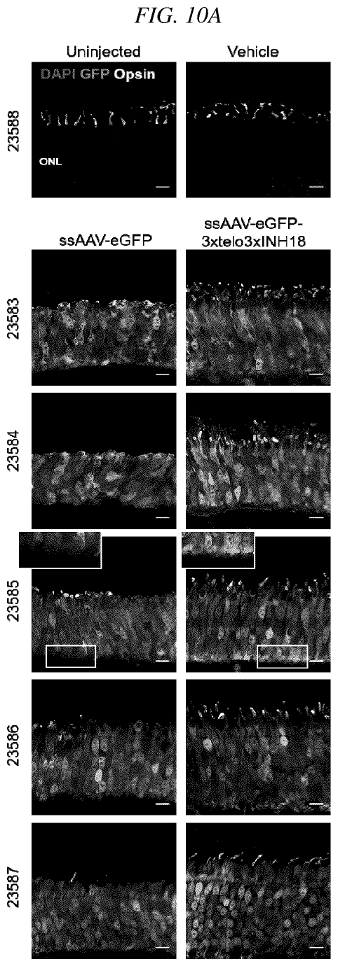

ssAAV-eGFP and ssAAV-eGFP-3xtelo3xINH18 were used and packaged in an AAV8 capsid (FIG. 4. Tables 2-4 and FIGS. 9A-9B). Endotoxin testing of AAV8 vectors using a limulus amebocyte lysate assay showed that both vectors were endotoxin free (<1 EU/ml) (Table 3). The percentage of full particles was determined by negatively staining AAV with 0.5% uranyl acetate and viewing under a transmission electron microscope. Empty particles show an electron-dense circle in the middle of the capsid (example shown with arrow in FIG. 9B). The number of empty and full particles was counted directly from electron micrographs of ten random images for each vector (Table 4). 1183 particles were counted for ssAAV-eGFP and 1221 particles were counted for ssAAV-eGFP-3xtelo3xINH18. The average percent full particles per image is reported with standard deviation. Representative images are shown for each vector with scale bar: 100 nm (FIG. 9B).

An intermediate dose of 4×10¹¹ vg was selected per eye based on a recently approved gene therapy for biallelic RPE65 mutation-associated retinal dystrophy (LUXTURNA™, dose=1.5×10¹¹ vg per eye) and previous reports showing ocular inflammation in patients at 1×10¹¹ vg and 1×10¹² vg per eye (Bainbridge et al., N Engl J Med 372, 1887-1897 (2015); Xue et al., Nat Med. 2018 Oct;24(10):1507-1512; Dimopoulos et al., Am J Ophthalmol. 2018Sep;193:130-142).

TABLE 2
Study Design.

| Animal | Dose | | |
|---|---|---|---|
| ID number | (vg)/eye | OD | OS |
| 23583 | 4 × 10¹¹ | ssAAV-eGFP- | ssAAV-eGFP |
| | | 3xtelo3xINH18 | |
| 23584 | 4 × 10¹¹ | ssAAV-eGFP- | ssAAV-eGFP |
| | | 3xtelo3xINH18 | |
| 23585 | 4 × 10¹¹ | ssAAV-eGFP- | ssAAV-eGFP |
| | | 3xtelo3xINH18 | |
| 23586 | 4 × 10¹¹ | ssAAV-eGFP- | ssAAV-eGFP |
| | | 3xtelo3xINH18 | |
| 23587 | 4 × 10¹¹ | ssAAV-eGFP- | ssAAV-eGFP |
| | | 3xtelo3xINH18 | |
| 23588 | N.A. | Vehicle | Uninjected |

TABLE 3
Endotoxin testing (LAL assay).

| Vector | EU/ml |
|---|---|
| ssAAV-GFP | <1.0 |
| ssAAV-eGFP-3xtelo3xINH18 | <1.0 |

TABLE 4
Percent full particles.

| Vector | % full |
|---|---|
| ssAAV-eGFP | 90.97 ± 3.36 |
| ssAAV-eGFP- | 91.08 ± 2.11 |
| 3xtelo3xINH18 | |

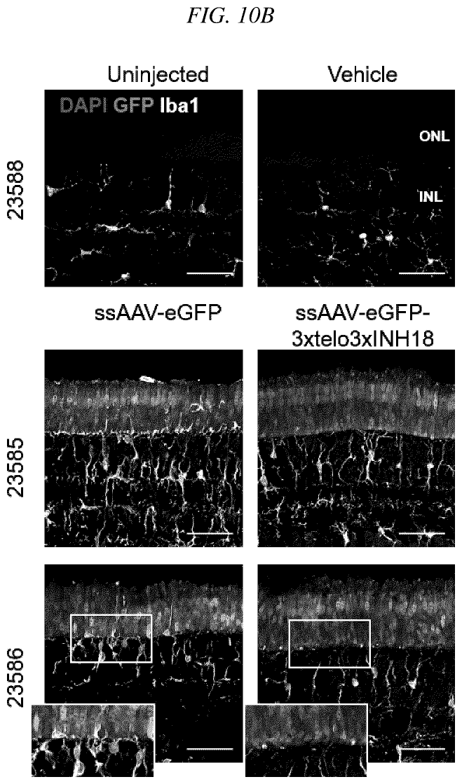

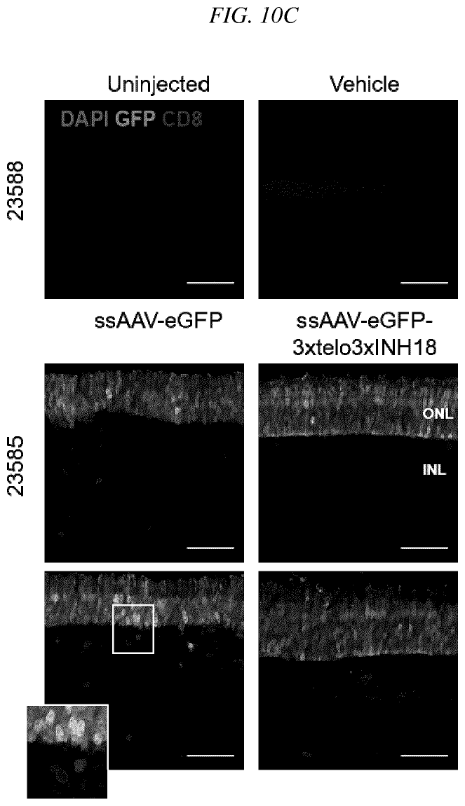

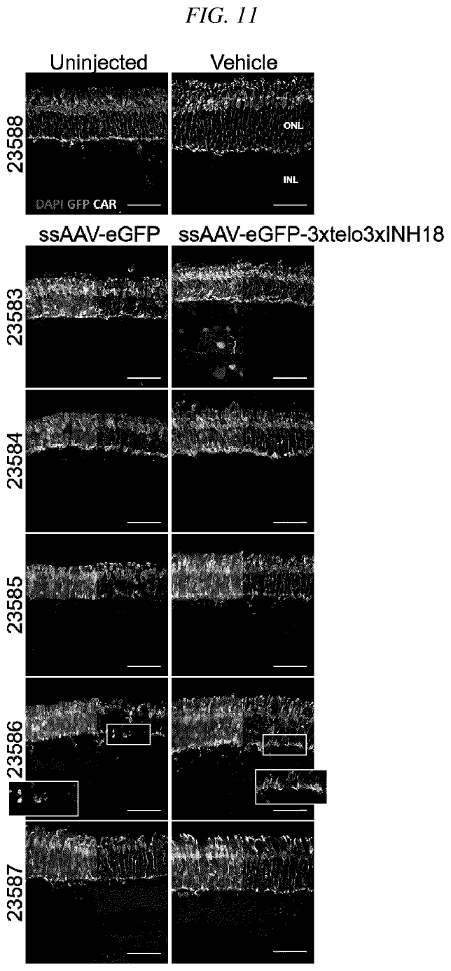

Using red-green opsin staining, it was observed that all five AAV-injected outbred pigs. ssAAV-eGFP led to a marked loss, shortening, or altered morphology of cone outer segments, suggesting AAV-induced pathology in the cone photoreceptors which are critical for high-acuity vision (FIG. 10A). In contrast, contralateral eyes injected with ssAAV-eGFP-3xtelo3xINH18 showed substantially better preservation of cone outer segments and appeared morphologically closer to uninjected or vehicle-injected eyes. These findings were confirmed by performing cone arrestin staining which labels the entire cone photoreceptor (FIG. 11). In addition, in two of five animals—animals 23585 and 23586—substantial loss or retraction of cone pedicles (the synaptic terminals of cone photoreceptors important for transferring the light signal onto the dendrites of bipolar cells and horizontal cells) was observed with ssAAV-eGFP; no such loss was observed in animals injected with ssAAV-eGFP-3xtelo3xINH18 (see FIG. 10A and FIG. 11).

Retinal images from in vivo optical coherence tomography (OCT) b-scans for damage to outer retinal lamination. As used in this example, OCT indicates Optical coherence tomography, RPE indicates retinal pigment epithelium, IS/OS indicates inner segment/outer segment, and wpi indicates weeks post injection. Representative OCT b-scan measurements in a vehicle-treated eye and from two different pigs whose left eye was injected with ssAAV-eGFP (OS) and whose right eye was injected with ssAAV-eGFP-3xtelo3xINH18 (OD) are shown in Table 5. In areas without damage, the outermost hyper-reflective band (located at the bottom of each b-scan, data not shown) represents the choroid/sclera. Moving inward, the next hyper-reflective band represents the RPE and the third band the IS/OS of the photoreceptors (data not shown). Severe damage were areas where both the photoreceptor and RPE layers were disrupted, non-severe damage were areas where the hyper-reflective bands for one or both of these layers were thinner and less well defined. Severe damage always surrounded the retinotomy and non-severe damage always surrounded areas of severe damage. Areas outside of the calipers had normal retinal lamination. The length of each type of damage (mm) is indicated in Table 5. Two length values for a particular type of damage indicate that there were two areas with that particular type of damage. Scale bars were used in each b-scan as controls (data not shown). The fundus image indicated the location of each b-scan on the retina (data not shown). Images at 2 (left) and 6 wpi (right) were used for each eye to determine whether damage changed with time after injection. At 2 wpi, the vehicle-treated eye had retinal detachment created by the subretinal injection, which was smaller at 6 wpi. Only animals 23586 and 23587 were analyzed as b-scans were available for most of the GFP+ region (mean=30 mm²) for all eyes. Animal 23583 was excluded as the area of GFP+ region was unusually small (1.4 mm² in each eye) and there was concern that the entire inoculum volume had not been deposited subretinally.

TABLE 5
Non-limiting example of types of damage observed in
the retina as measured by representative OCT b-scans.

| | 2 wpi | | | 6 wpi | | |
|---|---|---|---|---|---|---|
| | | Non-severe | Severe | | Non-severe | Severe |
| | Retinal | Laminar | Laminar | Retinal | Laminar | Laminar |
| | Detachment | Disruption | Damage | Detachment | Disruption | Damage |
| Treatment and | 2.493 mm | 0.964 mm | 0.665 mm | 0.834 mm; | 1.041 mm; | 1.468 mm |
| Animal Vehicle | | | | 0.840 mm | 0.523 mm | |
| Injection: | | | | | | |
| 23588 OD | | | | | | |
| ssAAV-eGFP: | n/a | 1.77 mm | 3.658 mm | n/a | 1.78 mm | 4.083 mm |
| 23586 OS | | | | | | |
| ssAAV-eGFP- | n/a | 1.569 mm | 2.394 mm | n/a | 1.266 mm; | 1.283 mm |
| 3xtelo3xINH18: | | | | | 3.158 mm | |
| 23586 OD | | | | | | |
| ssAAV-eGFP: | n/a | 2.389 mm; | 2.947 mm | n/a | 4.508 mm; | 3.208 mm |
| 23587 OS | | 0.763 mm | | | 0.804 mm | |
| ssAAV-eGFP- | n/a | n/a | 6.598 mm | n/a | 3.155 mm; | 0.858 mm |
| 3xtelo3xINH18: | | | | | 2.605 mm | |
| 23587 OD | | | | | | |

Summary data for the extent of each type of damage (see description of Table 5 above) were shown on fundus images for eyes injected subretinally with vehicle or two pigs whose left eye was injected with ssAAV-eGFP (OS) and whose right eye was injected with ssAAV-eGFP-3xtelo3xINH18 (OD) (fundus images not shown). Superimposed on each fundus image were the areas of severe and non-severe damage that were determined from OCT b-scans at 2 and at 6 wpi. Retinal areas with normal outer retina lamination are indicated in green. Severe damage always surrounded the retinotomy and was defined as loss of the hyper-reflective outer retinal bands that represent the RPE and photoreceptor inner/outer segments. Non-severe damage was usually found surrounding areas of severe damage and was defined as areas where the outer retinal hyper-reflective bands were thinner and/or more poorly defined. The fluorescence images of each eye cup were used and the GFP+ boundary was then superimposed on the fundus images. This analysis demonstrated that the areas of damage correspond to the GFP+ areas. In the eyes injected with ssAAV-eGFP-3xtelo3xINH18, the area of severe damage decreased between 2 and 6 wpi (˜84% smaller). In contrast, in the eyes injected with ssAAV-eGFP, the area with severe damage tended to remain the same.

It was found that outer retinal laminar pathology measured by OCT was consistent with retinal histology and that engineered vector comprising inhibitory oligonucleotide sequences ameliorated outer retina laminar pathology in subretinal-injected pig eyes. The vehicle-injected eye showed a small area of non-severe damage surrounding a similarly sized area of severe damage around the retinotomy site and the areas of damage were reduced between 2 wpi and 6 wpi (e.g., compare 2 wpi measurements with 6 wpi measurements for Vehicle Injection: 23588 OD in Table 5 and fundus images described above). In this eye, retinal detachment created by the subretinal injection was noted. For two animals (23586 and 23587) where b-scans were available for most of the GFP+ region for both eyes, similar damage between the two eyes at 2 wpi was observed (e.g., Table 5 below) However, at 6 wpi, the area of severe damage was largely reduced for both ssAAV-eGFP-3xtelo3xINH18 eyes, but not the two ssAAV-eGFP-treated eyes (e.g., severe laminar damage measurements at 6 wpi in Table 5).

Together, these data demonstrate that subretinal administration of higher doses of AAV can trigger photoreceptor pathology and that the engineered vector significantly reduces the induction of such pathology.

Next the immune responses in the retina were studied. Microglia cells are the resident innate immune cells of the retina and various reports in the literature suggest that CNS microglia can respond to CpG ligands (Olson et al., J Immunol 173, 3916-3924 (2004)). As expected, Iba1 staining of uninjected and vehicle-injected eyes showed a ramified staining pattern outside the outer nuclear layer (ONL), consistent with resting microglia (FIG. 10B). It was found that Iba1 signal increased significantly in all AAV-injected eyes (FIG. 10B and data not shown), suggesting microglia activation and proliferation, but only ssAAV-eGFP stimulated robust microglia infiltration into the ONL in animals 23585 and 23586, which was not seen with the engineered vector in the contralateral eyes. Furthermore, it was found that cytotoxic T cells infiltrating the outer and inner retina in the same two eyes that received ssAAV-eGFP, but not the contralateral ssAAV-eGFP-3xtelo3xINH18 eyes of the same animals or other AAV-treated animals. (FIG. 10C and data not shown),

A summary of the pig study findings, including histology and clinical examinations (inflammation scores) is included in Table 6. Immunohistochemical staining in the retina were performed 6 wpi at the terminus of the study. Vitritis (inflammation of the eye/retina via the SUN classification) was followed at weekly intervals from 2 wpi to 6 wpi. OCT imaging was performed at baseline (day of injection). 2 wpi and 6 wpi and area of damage to photoreceptor layers are shown for 6 wpi (terminus).

Taken together (see summary in Table 6), the engineered vector carrying the inhibitory oligonucleotide can evade eliciting undesirable innate immune and adaptive immune cell responses in the retina compared to the parental vector.

TABLE 6
Summary of pig study findings, including histology
and clinical examinations (inflammation scores).

| | | Substantial | Microg- | Cytotoxic | In-life |
|---|---|---|---|---|---|
| | | loss of cone | lia | T cell | vitritis |
| Animal ID | | outer | infil- | infil- | (score |
| number | Condition | segments | tration | tration | 0-5) |
| 23583 | ssAAV-eGFP | Yes | No | No | No |
| | ssAAV-eGFP- | No | No | No | No |
| | 3xtelo3xINH18 | | | | |
| 23584 | ssAAV-eGFP | Yes | No | No | No |
| | ssAAV-eGFP- | No | No | No | No |
| | 3xtelo3xINH18 | | | | |
| 23585 | ssAAV-eGFP | Yes* | Yes | Yes | Yes** |
| | ssAAV-eGFP- | No | No | No | No |
| | 3xtelo3xINH18 | | | | |
| 23586 | ssAAV-eGFP | Yes* | Yes | Yes | No |
| | ssAAV-eGFP- | No | No | No | No |
| | 3xtelo3xINH18 | | | | |
| 23587 | ssAAV-eGFP | Yes | No | No | No |
| | ssAAV-eGFP- | No | No | No | No |
| | 3xtelo3xINH18 | | | | |
| 23588 | Uninjected | N.A. | No | No | No |
| | Vehicle | N.A. | No | No | No |

*Loss of cone pedicles observed as well.
**3+ flare at 2 wpi, 1+ flare at 3 wpi, 0.5 flare at 4 wpi, traces of vitritis at 5 wpi, and vitritis resolved at 6 wpi. No steroid treatment was used throughout the course of vitritis.
wpi, week post-injection; SUN, Standardization of Uveitis Nomenclature; OCT, optical coherence tomography; N.A., not applicable.

### Example 10: Engineering a Single-Stranded AAV Vector and Testing Intravitreal Administration

Single-stranded AAV vectors ssAAV2-eGFP-WPRE (SEQ ID NO: 41) and ssAAV2-eGFP-WPRE-3xtelo3xINH18 (SEQ ID NO: 42) (FIG. 14) were administered intravitreally to the eyes of African Green monkey (one vector per eye). The engineered vector with the inhibitory oligonucleotide reduced the immune response relative to the control vector. For example, 2 weeks after intravitreal injection, animal A983, which received ssAAV-eGFP-WPRe in its OS eye had several signs of ocular inflammation by opthalmic examination, while the vehicle-injected OD eye did not (Table 7). Animal A982, which received ssAAV-eGFP-WPRE-3xtelo3xINH18 in its OS eye, did not have signs of ocular inflammation.

TABLE 7
Non-limiting example of inflammation
as measured by clinical examinations of the eye.

| | 2 wpi | | | |
|---|---|---|---|---|
| Treatment: | Anterior | Aqueous | Fibrin | Iris |
| Animal ID | chamber cells | flare | strands | hyperemia |
| and eye | score (0-4) | score (0-4) | score (0-3) | score 0-4) |
| 5 × 10¹¹ vg | 2 | 1 | 2 | 2 |
| ssAAV-eGFP- | | | | |
| WPRE: A983 OS | | | | |
| Vehicle | 0 | 0 | 0 | 0 |
| injection: | | | | |
| A983 OD | | | | |
| 5 × 10¹¹ vg | 0 | 0 | 0 | 0 |
| ssAAV-eGFP- | | | | |
| WPRE- | | | | |
| 3xtelo3xINH18: | | | | |
| A982 OS | | | | |
| Vehicle | 0 | 0 | 0 | 0 |
| injection: | | | | |
| A982 OD | | | | |

More specifically, in animal A983, the eye that was administered ssAAV-eGFP-WPRE (SEQ ID NO: 41) (control) showed greater inflammation following clinical examination compared to the eye in animal A982 that was administered ssAAV-eGFP-WPRE-3xtelo3xINH18 (SEQ ID NO: 42). The eye with control treatment (SEQ ID NO: 41) showed a moderate anterior chamber cell score with 11-20 cells, mild aqueous flare that was just detectable (i.e., the Tyndall effect was barely discernable; the intensity of the light beam in the anterior chamber was less than the intensity of the slit beam as it passes through the lens), moderated fibrin strands (large fibrinous strands/clots, or more than 5 small fibrinous strands), mild injection of tertiary vessels and minimal to moderate injection of the secondary vessels observed for the iris hyperemia score, moderate lens capsule deposits, and mild lens opacity or vacuoles/clefts between the lens fibers, and fundus detail was visible. In contrast, no such signs of inflammation were visible in the eye with ssAAV-eGFP-WPRE-3xtelo3xINH18 (SEQ ID NO: 42) administration.

### Example 11: Additional Engineering of Single-Stranded AAV Vector and Testing by Intramuscular Injection in Vivo in Mice

SSAAV-eGFP-3xtelo3xINH18 (packaged in rh32.33 capsid) showed reduced immune responses compared to ssAAV-eGFP and this occurs in an AAV dose-dependent fashion (FIG. 8A). Thus, to further reduce immunogenicity, 3 copies of 4084-F (3×SEQ ID NO: 3) were added with AAAAA (SEQ ID NO: 8) linkers followed by 3 copies of 2088 (3×SEQ ID NO: 2) with linkers but in the anti-sense direction between the left ITR and promoter (the 5′ untranslated region) of ssAAV-eGFP-3xtelo3xINH18 and term this ssAAV-eGFP-double. This vector, packaged in rh32.33 capsid, showed further reduction immune responses in mice upon intramuscular injection. Thus, it is possible to insert inhibitory oligonucleotides in various locations of the vector genome to reduce immune responses, and it is possible to further reduce immune responses by using more inhibitory oligonucleotides in combination.

### Materials and methods

C57BL/6 mice (male, 7-9 weeks old) were purchased from the Jackson Laboratory.

### AAV Vectors

Self-complementary (sc) were used in this study. Self-complementary vectors lack the terminal resolution sequence in one ITR. All vector genomes were flanked by AAV2 ITRs. scAAV-eGFP was purchased from Cell Biolabs (VPK-430) and has been previously described (Gray, JT et al., Methods Mol. Biol. 2011;807:25-46). scAAV-eGFP expressed enhanced green fluorescent protein (eGFP) from the cytomegalovirus (CMV) promoter, and included an SV40 intron and SV40 polyA sequence. The sequences of c41 oligonucleotide (5′-TGGCGCGCACCCACGGCCTG-3′; SEQ ID NO: 1) derived from Pseudomonas aeruginosa and telomere oligonucleotide (5′-TTTAGGGTTAGGGTTAGGGTTAGGG-3′; SEQ ID NO: 9; initial T nucleotide is optional for function) derived from mammalian telomeres have been described (Gursel, I et al., J Immunol. 2003;171(3):1393-400; Kaminski, JJ et al., J Immunol. 2013;191(7):3876-83; Shirota, H et al., J Immunol. 2005: 174 (8):4579-83; Li, Y et al., Vaccine. 2011;29(11):2193-8). A telomere oligonucleotide (manufactured by Invivogen, catalog code: tlrl-ttag) harbored an additional T (in bold) compared to published studies and thus was included in the sequence. During the course of this study. Invivogen removed the additional T in their manufactured telomere oligonucleotide (catalog code: tlrl-ttag151). In addition, control (5′-GCTAGATGTTAGCGT-3′; SEQ ID NO: 34) was used as a negative control sequence that does not inhibit TLR9 activation (Invivogen, catalog code: tlrl-2088c).

To engineer scAAV-eGFP, sequences were inserted into the unique SpeI site found immediately 5′ of the right ITR. To facilitate sub-cloning, a unique ClaI site was created immediately 5′ of the inserted sequences, thus allowing ClaI/SpeI sub-cloning of sequences. 3 copies of c41, telomere, or control oligonucleotide were inserted, separated by AAAAA (SEQ ID NO: 8) linkers, giving scAAV-eGFP-3xc41, scAAV-eGFP-3x telomere and scAAV-eGFP-3x control, respectively. Alternatively, one copy of telomere was inserted, with an AAAAA linker (SEQ ID NO: 8), giving scAAV-eGFP-1x telomere (data not shown).

Self-complementary vectors were packaged into AAV2 (Vigene Biosciences) by triple transfection of HEK293 cells and purified using iodixanol gradient ultracentrifugation and then concentrated to 500 ul using Amicon Ultra-15 columns in PBS. The purified viruses were titered by qPCR using primers derived from ITR and an AAV standard. The final yield of the viruses ranged from 0.5-3×10¹³ vg.

scAAV.FIX (Martino, AT et al. Blood 2011;117(24):6459-68) expressed human factor IX (FIX) under the control of a liver-specific transthyretin (TTR) mouse promoter and included a bovine growth hormone (bGH) polyA sequence. To engineer scAAV.FIX, sequences were inserted into the unique KpnI site found immediately 5′ of the TTR promoter. This vector was packaged into AAV8 by the core facility Gene Transfer Vector Core (GTVC) at Massachusetts Eye and Ear Infirmary (MEEI). The viral titers were determined by digital PCR using primers against the polyA sequence and total yield for each vector was calculated by multiplying viral titer (vg/ml) by volume.

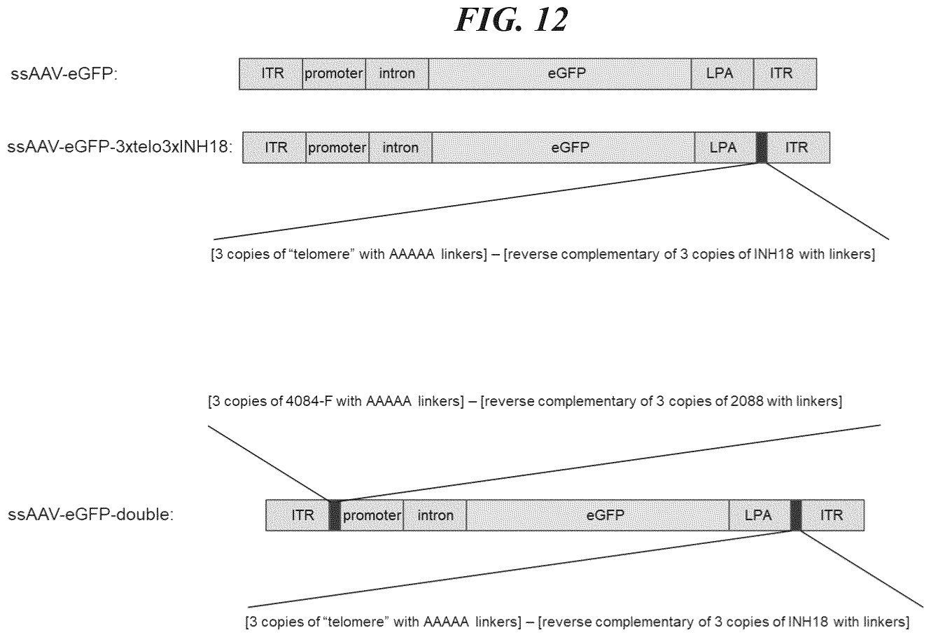

The single-stranded AAV vector ssAAV-eGFP has been previously described (Xiong, W. et al., J Clin Invest., 2015;125(4):1433-45) and was originally obtained from the Harvard DF/HCC DNA Resource Core (clone ID: EvNO00061595). ssAAV-eGFP contained a CMV enhancer/promoter, human β-globin intron, eGFP, and β-globin polyA sequence. To engineer ssAAV-eGFP, KpnI-5x telomere (sense)-5x telomere (anti-sense)-NheI was inserted immediately 5′ of the XbaI site adjacent to the right ITR. Again, AAAAA (SEQ ID NO: 8) was used as a linker between copies of the telomere sequence (SEQ ID NO: 9). Alternatively, KpnI-3x telomere (sense)-3xINH-18 (anti-sense)-NheI was inserted at the same site, similarly using AAAAA (SEQ ID NO: 8) as a linker between copies of the telomere sequence (SEQ ID NO: 9) or INH-18 (SEQ ID NO: 5). Both sense and anti-sense sequences of telomere (SEQ ID NO: 9) or INH-18 (SEQ ID NO: 5) were added as single-stranded AAV vectors have an equal chance of packaging positive or negative strands of the viral genome, thus ensuring that all packaged AAV genomes will carry copies of the telomere sequence (SEQ ID NO: 9) or INH-18 (SEQ ID NO: 5) in the right orientation. Finally. SpeI-3x 4084-F (sense)-3x2088 (anti-sense)-SpeI were additional inserted into the unique SpeI site found after the left ITR and before the promoter in ssAAV-eGFP-3xtelo3xINH18, and this vector was termed ssAAV-eGFP-double (FIG. 12). Single-stranded vectors were packaged into AAV8 (subretinal pig studies) or AAVrh32.33 (intramuscular mouse studies) and purified by the core facility Gene Transfer Vector Core (GTVC) at Massachusetts Eye and Ear Infirmary (MEEI). The viral titers were determined by digital PCR using primers against the CMV promoter and total yield for each vector was calculated by multiplying viral titer (vg/ml) by volume. The purity of vector preps was evaluated by running 1×10¹⁰ vg (viral genome) on an SDS-PAGE gel. In addition, vector preps had <1 EU/ml of endotoxin using a limulus amebocyte lysate assay (ToxinSensor Chromogenic LAL Endotoxin Assay Kit, Genscript). No significant differences in viral yield (viral titer x volume) were observed between parental vectors and corresponding engineered vectors for >20 purifications, suggesting that insertion of the described sequences do not hamper viral packaging.

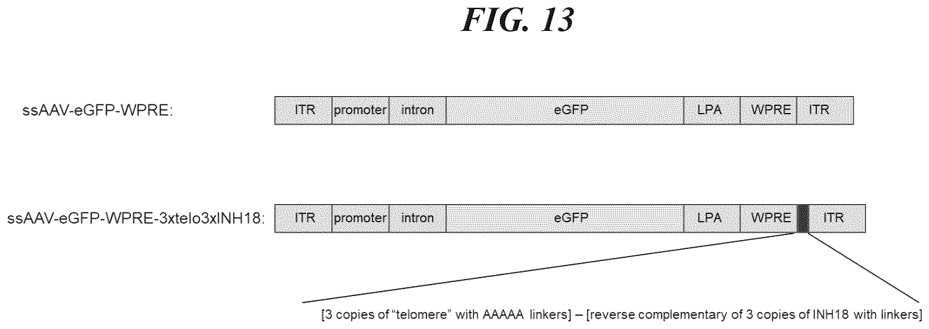

A different single-stranded vector, ssAAV-eGFP-WPRE contains a CMV promoter and woodchuck hepatitis virus posttranscriptional regulatory element (WPRE) for eGFP expression. To engineer ssAAV-eGFP-WPRE, KpnI-3x telomere (sense)-3xINH-18 (anti-sense) was inserted immediately 5′ of the XhoI site just upstream of the right ITR (FIG. 13). The parental vector and engineered vector were packaged into AAV2 and purified by cesium purification.

### HEK293-TLR9 Reporter Cell Line

A HEK293-based reporter cell line stably expressing human TLR9 and an inducible SEAP (secreted embryonic alkaline phosphatase) reporter gene was obtained (HEK-Blue hTLR9, Invivogen). The SEAP gene is under the control of the IFN-β minimal promoter fused to five NF-kB and AP-1 binding sites. Stimulation with a TLR9 ligand such as ODN 2006 activates NF-kB and AP-1 and therefore induces the production of SEAP, which can subsequently be measured to determine the amount of inflammation. All designed single-stranded DNA oligonucleotides were synthesized with a phosphorothioate backbone for increased stability (IDT). ODN 2006 was directly linked to indicated sequence with no intervening nucleotides, or with a AAAAA (SEQ ID NO: 8) linker sequence. ODN Control (15 nt) and ODN Control (24 nt) were 5′-TCC TGA GCT TGA AGT-3′ (SEQ ID NO: 43) and 5′-TTA TTA TTA TTA TTA TTA TTA TTA-3′ (SEQ ID NO: 44) respectively, and the two control sequences were selected to match the approximate range of lengths of the various TLR9 inhibitory oligonucleotides. Indicated concentrations of oligonucleotides were incubated with 6×10⁴ HEK293-TLR9 cells in 200 ul of DMEM growth media per well in 96-well flat bottom plates for 18 h, and 50 ul media was aspirated and incubated with 100 ul HEK-Blue Detection media (Invivogen) for 4-6 h at 37° C. and then absorbance at 639 nm was read on a plate reader. Similarly, HEK293 reporter cell lines stably expressing inducible SEAP reporter gene and human TLR7 (1×10⁵ cells) or TLR2 (6×10⁴ cells) (both from Invivogen) were stimulated with 1 μg/ml of Gardiquimod or 100 ng/ml of FSL-2 (both from Invivogen) respectively with or without control oligonucleotides or TLR9 inhibitory oligonucleotides for 18 h and SEAP activity was measured.

### Muscle Studies in Vivo

Adult C57BL/6 mice were injected intramuscularly with 50 ul PBS or AAV2 viruses (10¹¹ vg per animal, self-complementary vector) in the quadricep. 2 h later, the animals were sacrificed and a portion of the quadricep was saved in RNAlater solution (Thermo Scientific). The muscle tissues were subjected to RNA extraction, reverse transcription, and qPCR as described in the liver studies. For GFP expression studies, the quadricep was harvested 28 d later.

Similarly, adult mice were injected intramuscularly with 50 ul PBS or AAVrh32.33 viruses (10¹⁰ or 10¹¹ vg per animal, single-stranded vector) in the quadriceps. 21 d later, the animals were sacrified and spleens were harvested (see ELISPOT below) and muscle tisses were fixed in 10% formalin overnight and transferred to 70% ethanol. For histology, muscle samples were processed at DF/HCC Specialized Histopathology Services (SHS) and Beth Israel Deaconess Medical Center (BIDMC) histology core facilities, embedded in paraffin, and stained for CD8 (1:500, D4W2Z, CST) and granzyme B (1:500, polyclonal, catalog #AF1865, R&D). Antibodies were tagged with AlexaFluor 488 Tyramide (B40957, Thermo) or AlexaFluor 647 Tyramide (B40958, Thermo). All immunohistochemistry was performed on the Leica Bond automated staining platform using the Leica Biosystems Refine Detection Kit with citrate antigen retrieval. Muscle sections were also stained for GFP by immunohistochemistory (IHC) with IHC chromogen substrate DAB (3,3′-diaminobenzidine).

### IFN-γ T Cell ELISPOT Assays

Spleens were harvested from C57BL/6 mice injected i.m. with AAVrh32.33 single-stranded vectors 21 d post-injection. Spleens were passed through a 70 μm cell strainer (Fisher Scientific), and dissociated cells were spun down. The cell pellet was treated with 1 ml of ACK lysing buffer (Life Technologies) to lyse red blood cells. To determine the number of cells secreting IFN-γ in response to antigenic stimulation, an IFN-γ ELISPOT assay was used based on manufacturer's instructions (R&D Systems). Briefly, 96-well plates were pre-blocked with RPMI growth media for 2 h at room temperature and rinsed twice with PBS. 5×10⁵ splenocytes were seeded per well in T cell medium (DMEM supplemented with 10% heat-inactivated FBS, 1% penicillin/streptomycin, 1% L-glutamine, 10 mM HEPES buffer, 0.1 mM non-essential amino acids, 2 mM sodium pyruvate and 10⁻⁶ M beta-mercaptoethanol), with 2 μg/ml of a CD8+ h2-k^{b} restricted dominant epitope of AAVrh32.33 capsid (SSYELPYWM, purchased from Genemed Synthesis) or incubated with PMA/ionomycin as a non-specific positive control. ELISPOT plates were evaluated in blinded fashion (ZellNet Consulting, Inc., Fort Lee, NJ) using an automated Elispot reader system (KS ELISpot reader, Zeiss, Thornwood, USA) with KS ELISpot software version 4.9.16. The plate evaluation process including the setup of optimal reading parameters followed the International guidelines on Elispot plate evaluation⁶.

### Liver Studies in Vivo

Adult C57BL/6 mice were injected intravenously with 100 ul PBS or AAV2 viruses (10¹¹ vg per animal) or AAV8 viruses (10¹⁰ or 10¹¹ vg) by tail vein injection as previously described (Martino, AT et al. Blood 2011;117(24):6459-68) 2 h later, the animals were sacrificed and a portion of the right median lobe of the liver was saved in RNAlater solution (Thermo Scientific). Total RNA was extracted from 10-30 mg of mechanically disrupted liver sample by using an RNA extraction kit (OMEGA Bio-Tek). Similar amounts of RNA were reverse transcribed into cDNA with a high-capacity RNA-to-cDNA kit (Thermo Scientific) and similar amounts of cDNA were assayed with quantitative PCR (qPCR) using TaqMan Fast Advanced Master Mix (Thermo Scientific) and commercially available pre-designed primers/probes with FAM reporter dye for the indicated target genes (IDT). Expression level for each gene was calculated by normalizing against the housekeeping genes Actb or Gapdh using the ΔΔCT method and expressed as fold levels compared to saline-injected mice. All qPCR reactions were run on a realplex4 Mastercycle (Eppendorf). For GFP expression studies, the liver was harvested 14 d later. For human FIX expression, plasma (EDTA) were obtained 14 d and 28 d after AAV administration and human FIX expression was quantified by analyzing diluted plasma samples using an ELISA kit specific for human factor IX (ab188393, Abcam). Plasma from PBS-injected mice gave signals similar to that of a blank control, demonstrating specificity of the kit for human factor IX. The kit had a sensitivity of at least 0.78 ng/ml.

### Subretinal Injection of AAV in Domestic Pigs

All experimental protocols using pigs were performed at the University of Louisville, were approved by the University of Louisville Institutional Animal Care and Use Committee and adhered to the ARVO Statement for Use of Animals in Ophthalmic and Vision Research. Six 50 day old wild-type domestic female pigs were purchased (Oak Hill Genetics, Ewing Indiana). Surgery to inject AAV into the subretinal space was performed after a one week acclimatization period at the University of Louisville AALAC-approved facility. Details have been published previously⁷⁻⁹. Animals were sedated via intravenous administration of: Ketamine (10 mg/kg) and Dexmedetomidine (0.04 mg/kg) and treated with Atropine (0.25 mg/kg). An endotracheal tube was inserted and through it Isoflurane was administered to achieve a surgical plane of anesthesia (1-3%). An IV line inserted in the car vein was used to deliver IV fluids (Lactated Ringers Solution with or without 5% dextrose; 10-15 mL/kg/h) to maintain blood pressure and normal glycemic levels (60-140 mg/dL). Body temperature was monitored every 30 min with a rectal thermometer and maintained via a heated procedure table. Heart and respiration rates and oxygen saturation were recorded every 10 min throughout the procedures and anesthesia adjusted to maintain a normal range for these physiological parameters.

After anesthesia and aseptic surgical preparation, a vitreoretinal surgical approach was used to gain access to the subretinal space (between the retina and the pigment epithelium at the back of the eye) and to deposit an inoculum containing AAV in final formulation buffer (FFB) or FFB alone (vehicle). A lateral canthotomy was performed to increase exposure in the surgical field. After insertion of an eyelid speculum, two 25 g trocars were placed at 1.5 mm posterior to the limbus; one in superior-nasal and the other in inferior-nasal quadrant. An anterior chamber fluid paracentesis was performed to make space for the injected volume. A light pipe was inserted into one trochar to help visualize the retina. A 41 gauge subretinal cannula needle was placed through the other trochar and used to make a local retinal detachment (bleb) followed by injection of inoculum (˜75 μl). Either AAV8.GFP.io2 or AAV8.GFP was injected into OD or OS of two pigs (4 eyes) in each of three surgery sessions. After the injection, the light pipe, needle and the trochars were removed (they are self-sealing). The lateral canthotomy was sutured closed with 4-0 Nylon. Antibiotic and steroid ointment was placed topically at the end of the surgery.

From 2 weeks post-injection (wpi) to 6 wpi, a complete clinical examination assessed the health of the retina at weekly intervals in anesthetized pigs. This included a slit lamp examination to inspect the anterior segment of the eye and to characterize damage to the cornea/lens, indirect ophthalmoscopy to inspect the health of the fundus and fundus photography to document the state of the retina, its optic nerve, the blood vessel pattern, and any damage that resulted from surgical procedures or viral expression. In addition, each eye was scored for inflammation of the eye/retina using the SUN classification¹⁰. The retinal surgeon performing the injections was blinded to the test article, and similarly, clinical examinations and scoring of inflammation were performed blinded.

Prior to surgery and at 2 and 6 wpi, ocular coherence tomography (OCT; Bioptigen/Leica Biosciences) was performed to image the retinal layers in vivo. Pupils were dilated and accommodation relaxed with topical applications of 2.5% phenylephrine hydrochloride and 1% Tropicamide. Lid specula held the eyelids open and corneas were wet throughout the imaging with artificial tears (Tears Again, OcuSoft, Inc, Richmond, TX). Using the OCT b-scans, the retinotomy site was identified and lamination pattern of the hypo-and hyper-reflective bands was characterized as a function of distance from that area in both the axial and lateral dimensions. Two types of damage were identified: severe damage represented areas where the hyper-reflective bands representing the RPE and photoreceptor inner/outer segments were disrupted. Non-severe damage were areas where these hyper-reflective bands were present, but were thinner and thus, less well defined than areas without damage. The shape, location and size of these two types of damage were measured using the software provided with the OCT system. Specifically, calipers were placed over severe and flanking non-severe damage in b-scans across the fundus and their areas were computed and summed over the entire extent of damage. These areas were then superimposed on the fundus image and the areas of damage compared to the areas of GFP expression (see below).

At 6 wpi (terminus), pigs were anesthetized and killed with a solution of Beuthanasia (390 mg pentobarbital sodium, 50 mg phenytoin sodium/ml; 1 mL/5 kg), and their eyes were enucleated. The cornea and lens were removed and the eyecup was dissected and fixed in 4% paraformaldehyde in PBS for 1 h at room temperature and then washed in PBS. Wholemount retinas were examined using a low power fluorescent microscope (Olympus MVX10) and the region of GFP+ expression was located and images acquired and plotted on the fundus images relative to the blood vessels and optic nerve head. The retina was dissected so that the piece used for histology included all of the GFP+ region, as well as GFP-flanking regions.

The pig retinal tissue was cryoprotected in graded sucrose solutions up to 30% sucrose in PBS, then embedded in a 1:1 mixture of 30% sucrose and optimal cutting temperature (OCT) compound (Tissue-Tek) followed by cryosectioning on a Leica CM3050S (Leica Microsystems). Transverse sections of retinal tissue were cut at 20 μm. For immunohistochemistry, tissue sections were first blocked with 5% donkey serum (if the secondary antibody was donkey-origin) or 5% goat serum (if the secondary antibody was goat-origin) in PBS with 0.1% Triton X-100 for 1 h at room temperature. Sections were then stained overnight at 4° C. in blocking solution with primary antibodies against red-green opsin (1:600, AB5405, EMD Millipore), human cone arrestin¹¹ (1:10000), Iba1 (1:200, ab5076, Abcam) and CD8 (1:200, MCA1223GA, Bio-Rad), followed by staining for 2 h at room temperature with goat anti-rabbit, donkey anti-mouse, or donkey anti-goat AlexaFluor 594-labeled secondary antibodies (111-585-144, 715-585-150 and 705-586-147, all from Jackson ImmunoResearch) used at 1:1000 in PBS. Tissues were lastly stained with 4′,6-diamidino-2-phenylindole (DAPI) for 5 min and mounted using Fluoromount-G (Southern Biotech). The slides were examined using a LSM710 laser scanning confocal microscope (Zeiss) with a 40× oil-immersion objective, and image processing was performed using ZEN software and ImageJ. For sections from AAV-injected eyes, care was taken to acquire images of GFP+ regions near, but not directly at, the retinotomy scar (where there is damage to photoreceptors from the injection). Similar laser settings were used when acquiring images of the two eyes of each animal.

### Intravitreal Injection of AAV in African Green Monkeys

African Green monkeys received intravitreal injections of 100 ul of ssAAV-eGFP-WPRE or ssAAV-eGFP-WPRE-3xtelo3xINH18 (both packaged in AAV2 capsid) in one eye, and the vehicle control (buffer) in the other eye. Ophthalmic examinations by slit lamp biomicroscopy and retinoscopy, as well as fundus imaging (both color photos and fluorescence photos), were performed at various time points. Vitreous haze was graded on a scale of 0 to 4 using the Nussenblatt scale, and anterior chamber cells and aqueous flare were graded using a modified Hackett-McDonald scoring system. Optical coherence tomography (OCT) was also performed to image the retina. At termins, animals were euthanized and aqueous humor collected from the eyes. The enucleated eyes were fixed in 4% paraformaldehyde followed by sectioning and histological analysis for pathology and cellular immune responses.

### Statistics

Unpaired two-tailed Student's t-tests were used to compare differences between two unpaired experimental groups in most cases. A two-tailed Mann-Whitney test was used for some in vivo studies as indicated. A P value of <0.05 was considered statistically significant. No pre-specified effect size was assumed and in general three to ten replicates or animals for each condition was used.

Sequences
c41 oligonucleotide sequence:
(SEQ ID NO: 1)
TGGCGCGCACCCACGGCCTG.
ODN 2088:
(SEQ ID NO: 2)
TCC TGG CGG GGA AGT
ODN 4084-F:
(SEQ ID NO: 3)
CCTGGATGGGAA
ODN INH-1:
(SEQ ID NO: 4)
CCTGGATGGGAATTCCCATCCAGG
ODN INH-18:
(SEQ ID NO: 5)
CCT GGA TGG GAA CTT ACC GCT GCA
ODN TTAGGG:
(SEQ ID NO: 6)
TT AGG GTT AGG GTT AGG GTT AGG G
G-ODN:
(SEQ ID NO: 7)
CTC CTA TTG GGG GTT TCC TAT
linker sequence
(SEQ ID NO: 8)
AAAAA
telomere:
(SEQ ID NO: 9)
TTTAGGGTTAGGGTTAGGGTTAGGG
ODN 4137:
(SEQ ID NO: 10)
TCCTGGAGGGGAACC.
ODN 4033:
(SEQ ID NO: 11)
CCTGGAGGGGAAGT.
ODN 4171:
(SEQ ID NO: 12)
CCTGGAGGGG.
ODN 4352:
(SEQ ID NO: 13)
TCCTTCCTGGAGGGGAAG.
ODN 4191:
(SEQ ID NO: 14)
TCCTATCCTGGAGGGGAAG.
ODN 4351:
(SEQ ID NO: 15)
TCCTATCCTATCCTGGAGGGGAAG.
ODN 2114:
(SEQ ID NO: 16)
TCCTGGAGGGGAAGT
ODN 4024:
(SEQ ID NO: 17)
TCCTGGATGGGAAGT
ODN INH-4:
(SEQ ID NO: 18)
TTCCCATCCAGGCCTGGATGGGAA
ODN INH-13:
(SEQ ID NO: 19)
CTTACCGCTGCACCTGGATGGGAA
ODN Poly-G:
(SEQ ID NO: 20)
GGGGGGGGGGGGGGGGGGGG
ODN GpG:
(SEQ ID NO: 21)
TGACTGTGAAGGTTAGAGATGA
ODN IRS-869:
(SEQ ID NO: 22)
TCCTGGAGGGGTTGT
ODN IRS-954:
(SEQ ID NO: 23)
TGCTCCTGGAGGGGTTGT
ODN 21158:
(SEQ ID NO: 24)
CCTGGCGGGG.
ODN super:
(SEQ ID NO: 25)
CCTCAATAGGGTGAGGGG.
ODN 2006:
(SEQ ID NO: 26)
Tcgtcgttttgtcgttttgtcgtt.
ODN 4348:
(SEQ ID NO: 27)
TCGTATCCTGGAGGGGAAG.
ODN 4349:
(SEQ ID NO: 28)
TAATATCCTGGAGGGGAAG.
ODN 4347:
(SEQ ID NO: 29)
CCTATCCTGGAGGGGAAG.
ODN A:
(SEQ ID NO: 30)
GGGTGGGTGGGTATTACCATTA.
ODN B:
(SEQ ID NO: 31)
TGGGCGGTTCAACCTTCA.
ODN C:
(SEQ ID NO: 32)
CCTCAAGCTTGAGGGG.
scAAV-FIX:
(SEQ ID NO: 33)
gcgcgctcgctcgctcactgaggccgcccgggcaaagcccgggcgtcgggcgacctttggtcgcccggcctcagtgagcgagcgagc
gcgcagagagggagtggccaactccatcactaggggttccttgtagttaatgattaacccgccatgctacttatctacgtagccatgctcgat
ctgaattcggtaccacgcgtgtctgtctgcacatttcgtagagcgagtgttccgatactctaatctccctaggcaaggttcatatttgtgtaggtt
acttattctccttttgttgactaagtcaataatcagaatcagcaggtttggagtcagcttggcagggatcagcagcctgggttggaaggaggg
ggtataaaagccccttcaccaggagaagccgtcacacagatccacaagctcctgctagcaggtaagtgccgtgtgtggttcccgcgggcc
tggcctctttacgggttatggcccttgcgtgccttgaattactgacactgacatccactttttctttttctccacaggtatcgattgaattccaccat
gcagcgcgtgaacatgatcatggcagaatcaccaggcctcatcaccatctgccttttaggatatctactcagtgctgaatgtacagtttttcttg
atcatgaaaacgccaacaaaattctgaatcggccaaagaggtataattcaggtaaattggaagagtttgttcaagggaaccttgagagagaa
tgtatggaagaaaagtgtagttttgaagaagcacgagaagtttttgaaaacactgaaagaacaactgaattttggaagcagtatgttgatgga
gatcagtgtgagtccaatccatgtttaaatggcggcagttgcaaggatgacattaattcctatgaatgttggtgtccctttggatttgaaggaaa
gaactgtgaattagatgtaacatgtaacattaagaatggcagatgcgagcagttttgtaaaaatagtgctgataacaaggtggtttgctcctgta
ctgagggatatcgacttgcagaaaaccagaagtcctgtgaaccagcagtgccatttccatgtggaagagtttctgtttcacaaacttctaagct
cacccgtgctgagactgtttttcctgatgtggactatgtaaattctactgaagctgaaaccattttggataacatcactcaaagcacccaatcatt
taatgacttcactcgggttgttggtggagaagatgccaaaccaggtcaattcccttggcaggttgttttgaatggtaaagttgatgcattctgtg
gaggctctatcgttaatgaaaaatggattgtaactgctgcccactgtgttgaaactggtgttaaaattacagttgtcgcaggtgaacataatatt
gaggagacagaacatacagagcaaaagcgaaatgtgattcgaattattcctcaccacaactacaatgcagctattaataagtacaaccatga
cattgcccttctggaactggacgaacccttagtgctaaacagctacgttacacctatttgcattgctgacaaggaatacacgaacatcttcctc
aaatttggatctggctatgtaagtggctggggaagagtcttccacaaagggagatcagctttagttcttcagtaccttagagttccacttgttga
ccgagccacatgtcttcgatctacaaagttcaccatctataacaacatgttctgtgctggcttccatgaaggaggtagagattcatgtcaagga
gatagtgggggaccccatgttactgaagtggaagggaccagtttcttaactggaattattagctggggtgaagagtgtgcaatgaaaggca
aatatggaatatataccaaggtatcccggtatgtcaactggattaaggaaaaaacaaagctcacttaatgaaagatggagatctgcggcctc
gactagagctcgctgatcagcctcgactgtgccttctagttgccagccatctgttgtttgcccctcccccgtgccttccttgaccctggaaggt
gccactcccactgtcctttcctaataaaatgaggaaattgcatcgcattgtctgagtaggtgtcattctattctggggggtggggtggggcagg
acagcaagggggaggattgggaagacaatagcaggaaccccactccctctctgcgcgctcgctcgctcactgaggccgggcgaccaaa
ggtcgcccgacgcccgggctttgcccgggcggcctcagtgagcgagcgagcgcgcag.
scAAV-FIX-3xtelomere (inhibitory oligonucleotide in underlined and bold text):
(SEQ ID NO: 34)
gcgcgctcgctcgctcactgaggccgcccgggcaaagcccgggcgtcgggcgacctttggtcgcccggcctcagtgagcgagcgagc
gcgcagagagggagtggccaactccatcactaggggttccttgtagttaatgattaacccgccatgctacttatctacgtagccatgctcgat
ctgaattcggtacctttagggttagggttaguttagggAAAAAtttagggttaguttagggttagggAAAAAtttagggtt
agggttagggttagggAAAAAggtaccacgcgtgtctgtctgcacatttcgtagagcgagtgttccgatactctaatctccctaggcaa
ggttcatatttgtgtaggttacttattctccttttgttgactaagtcaataatcagaatcagcaggtttggagtcagcttggcagggatcagcagc
ctgggttggaaggagggggtataaaagccccttcaccaggagaagccgtcacacagatccacaagctcctgctagcaggtaagtgccgt
gtgtggttcccgcgggcctggcctctttacgggttatggcccttgcgtgccttgaattactgacactgacatccactttttctttttctccacaggt
atcgattgaattccaccatgcagcgcgtgaacatgatcatggcagaatcaccaggcctcatcaccatctgccttttaggatatctactcagtgc
tgaatgtacagtttttcttgatcatgaaaacgccaacaaaattctgaatcggccaaagaggtataattcaggtaaattggaagagtttgttcaag
ggaaccttgagagagaatgtatggaagaaaagtgtagttttgaagaagcacgagaagtttttgaaaacactgaaagaacaactgaattttgg
aagcagtatgttgatggagatcagtgtgagtccaatccatgtttaaatggcggcagttgcaaggatgacattaattcctatgaatgttggtgtcc
ctttggatttgaaggaaagaactgtgaattagatgtaacatgtaacattaagaatggcagatgcgagcagttttgtaaaaatagtgctgataac
aaggtggtttgctcctgtactgagggatatcgacttgcagaaaaccagaagtcctgtgaaccagcagtgccatttccatgtggaagagtttct
gtttcacaaacttctaagctcacccgtgctgagactgtttttcctgatgtggactatgtaaattctactgaagctgaaaccattttggataacatca
ctcaaagcacccaatcatttaatgacttcactcgggttgttggtggagaagatgccaaaccaggtcaattcccttggcaggttgttttgaatggt
aaagttgatgcattctgtggaggctctatcgttaatgaaaaatggattgtaactgctgcccactgtgttgaaactggtgttaaaattacagttgtc
gcaggtgaacataatattgaggagacagaacatacagagcaaaagcgaaatgtgattcgaattattcctcaccacaactacaatgcagctat
taataagtacaaccatgacattgcccttctggaactggacgaacccttagtgctaaacagctacgttacacctatttgcattgctgacaaggaa
tacacgaacatcttcctcaaatttggatctggctatgtaagtggctggggaagagtcttccacaaagggagatcagctttagttcttcagtacct
tagagttccacttgttgaccgagccacatgtcttcgatctacaaagttcaccatctataacaacatgttctgtgctggcttccatgaaggaggta
gagattcatgtcaaggagatagtgggggaccccatgttactgaagtggaagggaccagtttcttaactggaattattagctggggtgaagag
tgtgcaatgaaaggcaaatatggaatatataccaaggtatcccggtatgtcaactggattaaggaaaaaacaaagctcacttaatgaaagat
ggagatctgcggcctcgactagagctcgctgatcagcctcgactgtgccttctagttgccagccatctgttgtttgcccctcccccgtgccttc
cttgaccctggaaggtgccactcccactgtcctttcctaataaaatgaggaaattgcatcgcattgtctgagtaggtgtcattctattctggggg
gtggggtggggcaggacagcaagggggaggattgggaagacaatagcaggaaccccactccctctctgcgcgctcgctcgctcactga
ggccgggcgaccaaaggtcgcccgacgcccgggctttgcccgggcggcctcagtgagcgagcgagcgcgcag.
“3xtelomere” oligo:
(SEQ ID NO: 35)
tttagggttagggttagggttagggAAAAAtttagggttagggttagggttagggAAAAAtttagggttagggttagggttagggA
AAAA.
ssAAV-eGFP:
(SEQ ID NO: 36)
tgcgcgctcgctcgctcactgaggccgcccgggcaaagcccgggcgtcgggcgacctttggtcgcccggcctcagtgagcgagcgag
cgcgcagagagggagtggccaactccatcactaggggttccttgtagttaatgattaacccgccatgctacttatctacgtagccatgctcta
gcggcctcggcctctgcataaataaaaaaaattagtcagccatgagcttggcccattgcatacgttgtatccatatcataatatgtacatttatat
tggctcatgtccaacattaccgccatgttgacattgattattgactagttattaatagtaatcaattacggggtcattagttcatagcccatatatgg
agttccgcgttacataacttacggtaaatggcccgcctggctgaccgcccaacgacccccgcccattgacgtcaataatgacgtatgttccc
atagtaacgccaatagggactttccattgacgtcaatgggtggagtatttacggtaaactgcccacttggcagtacatcaagtgtatcatatgc
caagtacgccccctattgacgtcaatgacggtaaatggcccgcctggcattatgcccagtacatgaccttatgggactttcctacttggcagt
acatctacgtattagtcatcgctattaccatggtgatgcggttttggcagtacatcaatgggcgtggatagcggtttgactcacggggatttcca
agtctccaccccattgacgtcaatgggagtttgttttggcaccaaaatcaacgggactttccaaaatgtcgtaacaactccgccccattgacg
caaatgggcggtaggcgtgtacggtgggaggtctatataagcagagctcgtttagtgaaccgtcagatcgcctggagacgccatccacgc
tgttttgacctccatagaagacaccgggaccgatccagcctcccctcgaagctgatcctgagaacttcagggtgagtctatgggacccttga
tgttttctttccccttcttttctatggttaagttcatgtcataggaaggggagaagtaacagggtacacatattgaccaaatcagggtaattttgcat
ttgtaattttaaaaaatgctttcttcttttaatatacttttttgtttatcttatttctaatactttccctaatctctttctttcagggcaataatgat
acaatgtatcatgcctctttgcaccattctaaagaataacagtgataatttctgggttaaggcaatagcaatatttctgcatataaatatttctgcat
ataaattgtaactgatgtaagaggtttcatattgctaatagcagctacaatccagctaccattctgcttttattttatggttgggataaggctggatt
attctgagtccaagctaggcccttttgctaatcatgttcatacctcttatcttcctcccacagctcctgggcaacgtgctggtctgtgtgctggccca
tcactttggcaaagaattccgcgggcccgggatccaccggtcgccaccatggtgagcaagggcgaggagctgttcaccggggtggtgcccatcct
ggtcgagctggacggcgacgtaaacggccacaagttcagcgtgtccggcgagggcgagggcgatgccacctacggcaagctgaccct
gaagttcatctgcaccaccggcaagctgcccgtgccctggcccaccctcgtgaccaccctgacctacggcgtgcagtgcttcagccgcta
ccccgaccacatgaagcagcacgacttcttcaagtccgccatgcccgaaggctacgtccaggagcgcaccatcttcttcaaggacgacgg
caactacaagacccgcgccgaggtgaagttcgagggcgacaccctggtgaaccgcatcgagctgaagggcatcgacttcaaggaggac
ggcaacatcctggggcacaagctggagtacaactacaacagccacaacgtctatatcatggccgacaagcagaagaacggcatcaaggt
gaacttcaagatccgccacaacatcgaggacggcagcgtgcagctcgccgaccactaccagcagaacacccccatcggcgacggccc
cgtgctgctgcccgacaaccactacctgagcacccagtccgccctgagcaaagaccccaacgagaagcgcgatcacatggtcctgctgg
agttcgtgaccgccgccgggatcactctcggcatggacgagctgtacaagtaaagcggccgctctagaggatccaagcttatcgataccgt
cgacctcgagggcccagatctaattcaccccaccagtgcaggctgcctatcagaaagtggtggctggtgtggctaatgccctggcccaca
agtatcactaagctcgctttcttgctgtccaatttctattaaaggttcctttgttccctaagtccaactactaaactgggggatattatgaagggcct
tgagcatctggattctgcctaataaaaaacatttattttcattgcaatgatgtatttaaattatttctgaatattttactaaaaagggaatgtgggagg
tcagtgcatttaaaacataaagaaatgaagagctagttcaaaccttgggaaaatacactatatcttaaactccatgaaagaaggtgaggctgc
aaacagctaatgcacattggcaacagcccctgatgcctatgccttattcatccctcagaaaaggattcaagtagaggcttgatttggaggttaa
agttttgctatgctgtattttacattacttattgttttagctgtcctcatgaatgtcttttcactacccatttgcttatcctgcatctctcagccttgac
tccactcagttctcttgcttagagataccacctttcccctgaagtgttccttccatgttttacggcgagatggtttctcctcgcctggccactcagcctt
agttgtctctgttgtcttatagaggtctacttgaagaaggaaaaacagggggcatggtttgactgtcctgtgagcccttcttccctgcctccccc
actcacagtgacccggaatccctcgacatctagagcatggctacgtagataagtagcatggcgggttaatcattaactacaaggaaccccta
gtgatggagttggccactccctctctgcgcgctcgctcgctcactgaggccgggcgaccaaaggtcgcccgacgcccgggctttgcccg
ggcggcctcagtgagcgagcgagcgc.
ssAAV-eGFP-3xtelo3xINH18 (inhibitory oligonucleotide in underlined and bold text):
(SEQ ID NO: 37)
tgcgcgctcgctcgctcactgaggccgcccgggcaaagcccgggcgtcgggcgacctttggtcgcccggcctcagtgagcgagcgag
cgcgcagagagggagtggccaactccatcactaggggttccttgtagttaatgattaacccgccatgctacttatctacgtagccatgctcta
gcggcctcggcctctgcataaataaaaaaaattagtcagccatgagcttggcccattgcatacgttgtatccatatcataatatgtacatttatat
tggctcatgtccaacattaccgccatgttgacattgattattgactagttattaatagtaatcaattacggggtcattagttcatagcccatatatgg
agttccgcgttacataacttacggtaaatggcccgcctggctgaccgcccaacgacccccgcccattgacgtcaataatgacgtatgttccc
atagtaacgccaatagggactttccattgacgtcaatgggtggagtatttacggtaaactgcccacttggcagtacatcaagtgtatcatatgc
caagtacgccccctattgacgtcaatgacggtaaatggcccgcctggcattatgcccagtacatgaccttatgggactttcctacttggcagt
acatctacgtattagtcatcgctattaccatggtgatgcggttttggcagtacatcaatgggcgtggatagcggtttgactcacggggatttcca
agtctccaccccattgacgtcaatgggagtttgttttggcaccaaaatcaacgggactttccaaaatgtcgtaacaactccgccccattgacg
caaatgggcggtaggcgtgtacggtgggaggtctatataagcagagctcgtttagtgaaccgtcagatcgcctggagacgccatccacgc
tgttttgacctccatagaagacaccgggaccgatccagcctcccctcgaagctgatcctgagaacttcagggtgagtctatgggacccttga
tgttttctttccccttcttttctatggttaagttcatgtcataggaaggggagaagtaacagggtacacatattgaccaaatcagggtaattttgcat
ttgtaattttaaaaaatgctttcttcttttaatatacttttttgtttatcttatttctaatactttccctaatctctttctttcagggcaataatgat
acaatgtatcatgcctctttgcaccattctaaagaataacagtgataatttctgggttaaggcaatagcaatatttctgcatataaatatttctgcat
ataaattgtaactgatgtaagaggtttcatattgctaatagcagctacaatccagctaccattctgcttttattttatggttgggataaggctggatt
attctgagtccaagctaggcccttttgctaatcatgttcatacctcttatcttcctcccacagctcctgggcaacgtgctggtctgtgtgctggccca
tcactttggcaaagaattccgcgggcccgggatccaccggtcgccaccatggtgagcaagggcgaggagctgttcaccggggtggtgcccatcct
ggtcgagctggacggcgacgtaaacggccacaagttcagcgtgtccggcgagggcgagggcgatgccacctacggcaagctgaccct
gaagttcatctgcaccaccggcaagctgcccgtgccctggcccaccctcgtgaccaccctgacctacggcgtgcagtgcttcagccgcta
ccccgaccacatgaagcagcacgacttcttcaagtccgccatgcccgaaggctacgtccaggagcgcaccatcttcttcaaggacgacgg
caactacaagacccgcgccgaggtgaagttcgagggcgacaccctggtgaaccgcatcgagctgaagggcatcgacttcaaggaggac
ggcaacatcctggggcacaagctggagtacaactacaacagccacaacgtctatatcatggccgacaagcagaagaacggcatcaaggt
gaacttcaagatccgccacaacatcgaggacggcagcgtgcagctcgccgaccactaccagcagaacacccccatcggcgacggccc
cgtgctgctgcccgacaaccactacctgagcacccagtccgccctgagcaaagaccccaacgagaagcgcgatcacatggtcctgctgg
agttcgtgaccgccgccgggatcactctcggcatggacgagctgtacaagtaaagcggccgctctagaggatccaagcttatcgataccgt
cgacctcgagggcccagatctaattcaccccaccagtgcaggctgcctatcagaaagtggtggctggtgtggctaatgccctggcccaca
agtatcactaagctcgctttcttgctgtccaatttctattaaaggttcctttgttccctaagtccaactactaaactgggggatattatgaagggcct
tgagcatctggattctgcctaataaaaaacatttattttcattgcaatgatgtatttaaattatttctgaatattttactaaaaagggaatgtgggagg
tcagtgcatttaaaacataaagaaatgaagagctagttcaaaccttgggaaaatacactatatcttaaactccatgaaagaaggtgaggctgc
aaacagctaatgcacattggcaacagcccctgatgcctatgccttattcatccctcagaaaaggattcaagtagaggcttgatttggaggttaa
agttttgctatgctgtattttacattacttattgttttagctgtcctcatgaatgtcttttcactacccatttgcttatcctgcatctctcagccttga
ctccactcagttctcttgcttagagataccacctttcccctgaagtgttccttccatgttttacggcgagatggtttctcctcgcctggccactcagcc
ttagttgtctctgttgtcttatagaggtctacttgaagaaggaaaaacagggggcatggtttgactgtcctgtgagcccttcttccctgcctccccc
actcacagtgacccggaatccctcgacaGGTACCtttagggttagggttagggttagggAAAAAtttagggttagggttagg
gttagggAAAAAtttagggttagggttagggttagggAAAAATGCAGCGGTAAGTTCCCATCCAGG
TTTTTTGCAGCGGTAAGTTCCCATCCAGGTTTTTTGCAGCGGTAAGTTCCCATC
CAGGTTTTTGCTAGCtctagagcatggctacgtagataagtagcatggcgggttaatcattaactacaaggaacccctagtg
atggagttggccactccctctctgcgcgctcgctcgctcactgaggccgggcgaccaaaggtcgcccgacgcccgggctttgcccgggc
ggcctcagtgagcgagcgagcgc.
ssAAV-eGFP-double (inhibitory oligonucleotides in underlined and bold text):
(SEQ ID NO: 38)
tgcgcgctcgctcgctcactgaggccgcccgggcaaagcccgggcgtcgggcgacctttggtcgcccggcctcagtgagcgagcgag
cgcgcagagagggagtggccaactccatcactaggggttccttgtagttaatgattaacccgccatgctacttatctacgtagccatgctcta
gcggcctcggcctctgcataaataaaaaaaattagtcagccatgagcttggcccattgcatacgttgtatccatatcataatatgtacatttatat
tggctcatgtccaacattaccgccatgttgacattgattattgactagtcctggatgggaaAAAAAcctggatgggaaAAAAAcc
tggatgggaaAAAAAACTTCCCCGCCAGGATTTTTACTTCCCCGCCAGGATTTTTACT
TCCCCGCCAGGATTTTTactagttattaatagtaatcaattacggggtcattagttcatagcccatatatggagttccgcgtta
cataacttacggtaaatggcccgcctggctgaccgcccaacgacccccgcccattgacgtcaataatgacgtatgttcccatagtaacgcca
atagggactttccattgacgtcaatgggtggagtatttacggtaaactgcccacttggcagtacatcaagtgtatcatatgccaagtacgcccc
ctattgacgtcaatgacggtaaatggcccgcctggcattatgcccagtacatgaccttatgggactttcctacttggcagtacatctacgtatta
gtcatcgctattaccatggtgatgcggttttggcagtacatcaatgggcgtggatagcggtttgactcacggggatttccaagtctccacccca
ttgacgtcaatgggagtttgttttggcaccaaaatcaacgggactttccaaaatgtcgtaacaactccgccccattgacgcaaatgggcggta
ggcgtgtacggtgggaggtctatataagcagagctcgtttagtgaaccgtcagatcgcctggagacgccatccacgctgttttgacctccat
agaagacaccgggaccgatccagcctcccctcgaagctgatcctgagaacttcagggtgagtctatgggacccttgatgttttctttcccctt
cttttctatggttaagttcatgtcataggaaggggagaagtaacagggtacacatattgaccaaatcagggtaattttgcatttgtaattttaaaa
aatgctttcttcttttaatatacttttttgtttatcttatttctaatactttccctaatctctttctttcagggcaataatgatacaatgtatcat
gcctctttgcaccattctaaagaataacagtgataatttctgggttaaggcaatagcaatatttctgcatataaatatttctgcatataaattgta
actgatgtaagaggtttcatattgctaatagcagctacaatccagctaccattctgcttttattttatggttgggataaggctggattattctgag
tccaagctaggcccttttgctaatcatgttcatacctcttatcttcctcccacagctcctgggcaacgtgctggtctgtgtgctggcccatcactt
tggcaaagaattccgcgggcccgggatccaccggtcgccaccatggtgagcaagggcgaggagctgttcaccggggtggtgcccatcctggtcgag
ctggacggcgacgtaaacggccacaagttcagcgtgtccggcgagggcgagggcgatgccacctacggcaagctgaccctgaagttcatctgc
accaccggcaagctgcccgtgccctggcccaccctcgtgaccaccctgacctacggcgtgcagtgcttcagccgctaccccgaccacat
gaagcagcacgacttcttcaagtccgccatgcccgaaggctacgtccaggagcgcaccatcttcttcaaggacgacggcaactacaagac
ccgcgccgaggtgaagttcgagggcgacaccctggtgaaccgcatcgagctgaagggcatcgacttcaaggaggacggcaacatcctg
gggcacaagctggagtacaactacaacagccacaacgtctatatcatggccgacaagcagaagaacggcatcaaggtgaacttcaagat
ccgccacaacatcgaggacggcagcgtgcagctcgccgaccactaccagcagaacacccccatcggcgacggccccgtgctgctgcc
cgacaaccactacctgagcacccagtccgccctgagcaaagaccccaacgagaagcgcgatcacatggtcctgctggagttcgtgaccg
ccgccgggatcactctcggcatggacgagctgtacaagtaaagcggccgctctagaggatccaagcttatcgataccgtcgacctcgagg
gcccagatctaattcaccccaccagtgcaggctgcctatcagaaagtggtggctggtgtggctaatgccctggcccacaagtatcactaag
ctcgctttcttgctgtccaatttctattaaaggttcctttgttccctaagtccaactactaaactgggggatattatgaagggccttgagcatctgg
attctgcctaataaaaaacatttattttcattgcaatgatgtatttaaattatttctgaatattttactaaaaagggaatgtgggaggtcagtgcat
ttaaaacataaagaaatgaagagctagttcaaaccttgggaaaatacactatatcttaaactccatgaaagaaggtgaggctgcaaacagctaat
gcacattggcaacagcccctgatgcctatgccttattcatccctcagaaaaggattcaagtagaggcttgatttggaggttaaagttttgctatg
ctgtattttacattacttattgttttagctgtcctcatgaatgtcttttcactacccatttgcttatcctgcatctctcagccttgactccactcagt
tctcttgcttagagataccacctttcccctgaagtgttccttccatgttttacggcgagatggtttctcctcgcctggccactcagccttagttgtct
ctgttgtcttatagaggtctacttgaagaaggaaaaacagggggcatggtttgactgtcctgtgagcccttcttccctgcctcccccactcacagtga
cccggaatccctcgacaGGTACCtttagggttagggttagggttagggAAAAAtttagggttagggttagggttagggAA
AAAtttagggttagggttagggttagggAAAAATGCAGCGGTAAGTTCCCATCCAGGTTTTTT
GCAGCGGTAAGTTCCCATCCAGGTTTTTTGCAGCGGTAAGTTCCCATCCAGGT
TTTTGCTAGCtctagagcatggctacgtagataagtagcatggcgggttaatcattaactacaaggaacccctagtgatggagttg
gccactccctctctgcgcgctcgctcgctcactgaggccgggcgaccaaaggtcgcccgacgcccgggctttgcccgggcggcctcagt
gagcgagcgagcgc.
“3xtelo3xINH18” oligo:
(SEQ ID NO: 39)
tttagggttagggttagggttagggAAAAAtttagggttagggttagggttagggAAAAAtttagggttagggttagggttagggA
AAAATGCAGCGGTAAGTTCCCATCCAGGTTTTTTGCAGCGGTAAGTTCCCATCCAGG
TTTTTTGCAGCGGTAAGTTCCCATCCAGGTTTTT.
“3x4084-F3x2088” oligo:
(SEQ ID NO: 40)
cctggatgggaaAAAAAcctggatgggaaAAAAAcctggatgggaaAAAAAACTTCCCCGCCAGGATTT
TTACTTCCCCGCCAGGATTTTTACTTCCCCGCCAGGATTTTT.
ssAAV-eGFP-WPRE:
(SEQ ID NO: 41)
ctgcgcgctcgctcgctcactgaggccgcccgggcaaagcccgggcgtcgggcgacctttggtcgcccggcctcagtgagcgagcga
gcgcgcagagagggagtggccaactccatcactaggggttccttgtagttaatgattaacccgccatgctacttatctacgtagccatgctct
aggaagatcggaattcgcccttaagctagctagttattaatagtaatcaattacggggtcattagttcatagcccatatatggagttccgcgtta
cataacttacggtaaatggcccgcctggctgaccgcccaacgacccccgcccattgacgtcaataatgacgtatgttcccatagtaacgcca
atagggactttccattgacgtcaatgggtggagtatttacggtaaactgcccacttggcagtacatcaagtgtatcatatgccaagtacgcccc
ctattgacgtcaatgacggtaaatggcccgcctggcattatgcccagtacatgaccttatgggactttcctacttggcagtacatctacgtatta
gtcatcgctattaccatggtgatgcggttttggcagtacatcaatgggcgtggatagcggtttgactcacggggatttccaagtctccacccca
ttgacgtcaatgggagtttgttttggcaccaaaatcaacgggactttccaaaatgtcgtaacaactccgccccattgacgcaaatgggcggta
ggcgtgtacggtgggaggtctatataagcagagctggtttagtgaaccgtcagatcctgcagaagttggtcgtgaggcactgggcaggtaa
gtatcaaggttacaagacaggtttaaggagaccaatagaaactgggcttgtcgagacagagaagactcttgcgtttctgataggcacctattg
gtcttactgacatccactttgcctttctctccacaggtgtccaggcggccgccatggtgagcaagggcgaggagctgttcaccggggtggtg
cccatcctggtcgagctggacggcgacgtaaacggccacaagttcagcgtgtccggcgagggcgagggcgatgccacctacggcaag
ctgaccctgaagttcatctgcaccaccggcaagctgcccgtgccctggcccaccctcgtgaccaccctgacctacggcgtgcagtgcttca
gccgctaccccgaccacatgaagcagcacgacttcttcaagtccgccatgcccgaaggctacgtccaggagcgcaccatcttcttcaagg
acgacggcaactacaagacccgcgccgaggtgaagttcgagggcgacaccctggtgaaccgcatcgagctgaagggcatcgacttcaa
ggaggacggcaacatcctggggcacaagctggagtacaactacaacagccacaacgtctatatcatggccgacaagcagaagaacggc
atcaaggtgaacttcaagatccgccacaacatcgaggacggcagcgtgcagctcgccgaccactaccagcagaacacccccatcggcg
acggccccgtgctgctgcccgacaaccactacctgagcacccagtccgccctgagcaaagaccccaacgagaagcgcgatcacatggt
cctgctggagttcgtgaccgccgccgggatcactctcggcatggacgagctgtacaagtaataagcttggatccaatcaacctctggattac
aaaatttgtgaaagattgactggtattcttaactatgttgctccttttacgctatgtggatacgctgctttaatgcctttgtatcatgctattgct
tcccgtatggctttcattttctcctccttgtataaatcctggttgctgtctctttatgaggagttgtggcccgttgtcaggcaacgtggcgtggtg
tgcactgtgtttgctgacgcaacccccactggttggggcattgccaccacctgtcagctcctttccgggactttcgctttccccctccctattgcc
acggcggaactcatcgccgcctgccttgcccgctgctggacaggggctcggctgttgggcactgacaattccgtggtgttgtcggggaaatcatc
gtcctttccttggctgctcgcctgtgttgccacctggattctgcgcgggacgtccttctgctacgtcccttcggccctcaatccagcggaccttc
cttcccgcggcctgctgccggctctgcggcctcttccgcgtcttcgagatctgcctcgactgtgccttctagttgccagccatctgttgtttgcc
cctcccccgtgccttccttgaccctggaaggtgccactcccactgtcctttcctaataaaatgaggaaattgcatcgcattgtctgagtaggtgt
cattctattctggggggtggggtggggcaggacagcaagggggaggattgggaagacaatagcaggcatgctggggactcgagttaag
ggcgaattcccgataaggatcttcctagagcatggctacgtagataagtagcatggcgggttaatcattaactacaaggaacccctagtgat
ggagttggccactccctctctgcgcgctcgctcgctcactgaggccgggcgaccaaaggtcgcccgacgcccgggctttgcccgggcg
gcctcagtgagcgagcgagcgcgcag
ssAAV-GFP-WPRE-3xtelo3xINH18 (inhibitory oligonucleotides in underlined and bold
text):
(SEQ ID NO: 42)
ctgcgcgctcgctcgctcactgaggccgcccgggcaaagcccgggcgtcgggcgacctttggtcgcccggcctcagtgagcgagcga
gcgcgcagagagggagtggccaactccatcactaggggttccttgtagttaatgattaacccgccatgctacttatctacgtagccatgctct
aggaagatcggaattcgcccttaagctagctagttattaatagtaatcaattacggggtcattagttcatagcccatatatggagttccgcgtta
cataacttacggtaaatggcccgcctggctgaccgcccaacgacccccgcccattgacgtcaataatgacgtatgttcccatagtaacgcca
atagggactttccattgacgtcaatgggtggagtatttacggtaaactgcccacttggcagtacatcaagtgtatcatatgccaagtacgcccc
ctattgacgtcaatgacggtaaatggcccgcctggcattatgcccagtacatgaccttatgggactttcctacttggcagtacatctacgtatta
gtcatcgctattaccatggtgatgcggttttggcagtacatcaatgggcgtggatagcggtttgactcacggggatttccaagtctccacccca
ttgacgtcaatgggagtttgttttggcaccaaaatcaacgggactttccaaaatgtcgtaacaactccgccccattgacgcaaatgggcggta
ggcgtgtacggtgggaggtctatataagcagagctggtttagtgaaccgtcagatcctgcagaagttggtcgtgaggcactgggcaggtaa
gtatcaaggttacaagacaggtttaaggagaccaatagaaactgggcttgtcgagacagagaagactcttgcgtttctgataggcacctattg
gtcttactgacatccactttgcctttctctccacaggtgtccaggcggccgccatggtgagcaagggcgaggagctgttcaccggggtggtg
cccatcctggtcgagctggacggcgacgtaaacggccacaagttcagcgtgtccggcgagggcgagggcgatgccacctacggcaag
ctgaccctgaagttcatctgcaccaccggcaagctgcccgtgccctggcccaccctcgtgaccaccctgacctacggcgtgcagtgcttca
gccgctaccccgaccacatgaagcagcacgacttcttcaagtccgccatgcccgaaggctacgtccaggagcgcaccatcttcttcaagg
acgacggcaactacaagacccgcgccgaggtgaagttcgagggcgacaccctggtgaaccgcatcgagctgaagggcatcgacttcaa
ggaggacggcaacatcctggggcacaagctggagtacaactacaacagccacaacgtctatatcatggccgacaagcagaagaacggc
atcaaggtgaacttcaagatccgccacaacatcgaggacggcagcgtgcagctcgccgaccactaccagcagaacacccccatcggcg
acggccccgtgctgctgcccgacaaccactacctgagcacccagtccgccctgagcaaagaccccaacgagaagcgcgatcacatggt
cctgctggagttcgtgaccgccgccgggatcactctcggcatggacgagctgtacaagtaataagcttggatccaatcaacctctggattac
aaaatttgtgaaagattgactggtattcttaactatgttgctccttttacgctatgtggatacgctgctttaatgcctttgtatcatgctattgct
tcccgtatggctttcattttctcctccttgtataaatcctggttgctgtctctttatgaggagttgtggcccgttgtcaggcaacgtggcgtggtg
tgcactgtgtttgctgacgcaacccccactggttggggcattgccaccacctgtcagctcctttccgggactttcgctttccccctccctattgcc
acggcggaactcatcgccgcctgccttgcccgctgctggacaggggctcggctgttgggcactgacaattccgtggtgttgtcggggaaatcatc
gtcctttccttggctgctcgcctgtgttgccacctggattctgcgcgggacgtccttctgctacgtcccttcggccctcaatccagcggaccttc
cttcccgcggcctgctgccggctctgcggcctcttccgcgtcttcgagatctgcctcgactgtgccttctagttgccagccatctgttgtttgcc
cctcccccgtgccttccttgaccctggaaggtgccactcccactgtcctttcctaataaaatgaggaaattgcatcgcattgtctgagtaggtgt
cattctattctggggggtggggtggggcaggacagcaagggggaggattgggaagacaatagcaggcatgctggggaGGTACCtt
tagggttagggttagggttagggAAAAAtttagggttagggttagggttagggAAAAAtttagggttagggttagggttag
ggAAAAATGCAGCGGTAAGTTCCCATCCAGGTTTTTTGCAGCGGTAAGTTCCCA
TCCAGGTTTTTTGCAGCGGTAAGTTCCCATCCAGGTTTTTctcgagttaagggcgaattcccga
taaggatcttcctagagcatggctacgtagataagtagcatggcgggttaatcattaactacaaggaacccctagtgatggagttggccactc
cctctctgcgcgctcgctcgctcactgaggccgggcgaccaaaggtcgcccgacgcccgggctttgcccgggcggcctcagtgagcga
gcgagcgcgcag
TTAGGG motif:
(SEQ ID NO: 61)
TTAGGG

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including.” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean ±10% of the recited numerical value.

Where a range of values is provided, each value between the upper and lower ends of the range are specifically contemplated and described herein.

1. A method comprising administering to a muscle tissue of a subject a recombinant viral genome comprising a therapeutic nucleotide sequence and an inhibitory oligonucleotide that inhibits nucleic acid-sensing toll-like receptor (TLR) activation and/or signaling, wherein the recombinant viral genome inhibits an inflammatory response to the recombinant viral genome in the muscle tissue.

2. The method of claim 1, wherein the TLR is TLR9.

3. The method of claim 1, wherein the recombinant viral genome is from adeno-associated virus (AAV), adenovirus, herpes simplex virus, varicella, variola virus, hepatitis B, cytomegalovirus, JC polyomavirus, BK polyomavirus, monkeypox virus, Herpes Zoster, Epstein-Barr virus, human herpes virus 7, Kaposi's sarcoma-associated herpesvirus, or human parvovirus B19.

4. The method of claim 3, wherein the recombinant viral genome is a recombinant AAV viral genome.

5. The method of claim 1, wherein the recombinant viral genome is single stranded.

6. The method of claim 1, wherein the recombinant viral genome is self-complementary.

7. The method of claim 1, wherein the inhibitory oligonucleotide is located upstream from (5′) a promoter operably linked to the therapeutic nucleotide sequence.

8. The method of claim 1, wherein the inhibitory oligonucleotide is located downstream from (3′) a polyA tail linked to the therapeutic nucleic acid.

9.-12. (canceled)

13. The method of claim 1, wherein the inhibitory oligonucleotide comprises a nucleotide sequence that shares 90% to 100% identity with the nucleotide sequence of any one of SEQ ID NOS: 1-7, 9-25, or 27-32.

14. The method of claim 1, wherein the inhibitory oligonucleotide comprises multiple tandem repeats.

15. The method of claim 1, wherein the inhibitory oligonucleotide comprises the nucleotide sequence of SEQ ID NO: 35.

16. The method of claim 1, wherein the inhibitory oligonucleotide comprises the nucleotide sequence of SEQ ID NO: 39.

17. (canceled)

18. The method of claim 1, wherein the inhibitory oligonucleotide comprises the nucleotide sequence of SEQ ID NO: 40 (3x4084-F3x2088).

19. The method of claim 1, wherein the inhibitory oligonucleotide comprises at least one, at least two, at least three, or at least four TTAGGG (SEQ ID NO: 61) motif(s).

20. The method of claim 1, the subject has a genetic disorder.

21. The method of claim 1, wherein the therapeutic nucleotide sequence encodes a therapeutic nucleic acid.

22.-24. (canceled)

25. A recombinant viral genome comprising a therapeutic nucleotide sequence and an inhibitory oligonucleotide that comprises the nucleotide sequence of any one of SEQ ID NOS: 2-5, 7, 10-25, or 27-32.

26.-35. (canceled)

36. The method of claim 7, wherein the inhibitor oligonucleotides is located in a 5′ untranslated region (UTR) of the recombinant viral genome.

37. The method of claim 8, wherein the inhibitor oligonucleotides is located in a 5′ untranslated region (UTR) of the recombinant viral genome.
